SEQUENCE LISTING (42 sequences)

SEQ ID NO: 1 - DNA, 21 bp, Artificial Sequence
  “A class” CpG oligonucleotide
ggggacgacgtcgtggggggg

SEQ ID NO: 2 - DNA, 21 bp, Artificial Sequence
  “A class” CpG oligonucleotide
ggggacgacgtcgtggggggg

SEQ ID NO: 3 - DNA, 21 bp, Artificial Sequence
  “B class” CpG oligonucleotide
tcgtcgtttttcggtgctttt

SEQ ID NO: 4 - DNA, 21 bp, Artificial Sequence
  “B class” CpG oligonucleotide
tcgtcgtttttcggtcgtttt

SEQ ID NO: 5 - DNA, 24 bp, Artificial Sequence
  “B class” CpG oligonucleotide
tcgtcgttttgtcgttttgtcgtt

SEQ ID NO: 6 - DNA, 24 bp, Artificial Sequence
  “B class” CpG oligonucleotide
tcgtcgtttcgtcgttttgtcgtt

SEQ ID NO: 7 - DNA, 24 bp, Artificial Sequence
  “B class” CpG oligonucleotide
tcgtcgttttgtcgtttttttcga

SEQ ID NO: 8 - DNA, 21 bp, Artificial Sequence
  B-Class oligonucleotide
tcgtcgtttttcggtgctttt

SEQ ID NO: 9 - DNA, 21 bp, Artificial Sequence
  B-Class oligonucleotide
tcgtcgtttttcggtcgtttt

SEQ ID NO: 10 - DNA, 24 bp, Artificial Sequence
  B-Class oligonucleotide
tcgtcgttttgtcgttttgtcgtt

SEQ ID NO: 11 - DNA, 24 bp, Artificial Sequence
  B-Class oligonucleotide
tcgtcgtttcgtcgttttgtcgtt

SEQ ID NO: 12 - DNA, 24 bp, Artificial Sequence
  B-Class oligonucleotide
tcgtcgttttgtcgtttttttcga

SEQ ID NO: 13 - DNA, 22 bp, Artificial Sequence
  C class CpG Oligonucleotide
tcgcgtcgttcggcgcgcgccg

SEQ ID NO: 14 - DNA, 23 bp, Artificial Sequence
  C class CpG Oligonucleotide
tcgtcgacgttcggcgcgcgccg

SEQ ID NO: 15 - DNA, 21 bp, Artificial Sequence
  C class CpG Oligonucleotide
tcggacgttcggcgcgcgccg

SEQ ID NO: 16 - DNA, 19 bp, Artificial Sequence
  C class CpG Oligonucleotide
tcggacgttcggcgcgccg

SEQ ID NO: 17 - DNA, 20 bp, Artificial Sequence
  C class CpG Oligonucleotide
tcgcgtcgttcggcgcgccg

SEQ ID NO: 18 - DNA, 20 bp, Artificial Sequence
  C class CpG Oligonucleotide
tcgacgttcggcgcgcgccg

SEQ ID NO: 19 - DNA, 18 bp, Artificial Sequence
  C class CpG Oligonucleotide
tcgacgttcggcgcgccg

SEQ ID NO: 20 - DNA, 18 bp, Artificial Sequence
  C class CpG Oligonucleotide
tcgcgtcgttcggcgccg

SEQ ID NO: 21 - DNA, 22 bp, Artificial Sequence
  C class CpG Oligonucleotide
tcgcgacgttcggcgcgcgccg

SEQ ID NO: 22 - DNA, 22 bp, Artificial Sequence
  C class CpG Oligonucleotide
tcgtcgttttcggcgcgcgccg

SEQ ID NO: 23 - DNA, 22 bp, Artificial Sequence
  C class CpG Oligonucleotide
tcgtcgttttcggcggccgccg

SEQ ID NO: 24 - DNA, 24 bp, Artificial Sequence
  C class CpG Oligonucleotide
tcgtcgttttacggcgccgtgccg

SEQ ID NO: 25 - DNA, 23 bp, Artificial Sequence
  C class CpG Oligonucleotide
tcgtcgttttcggcgcgcgccgt

SEQ ID NO: 26 - DNA, 22 bp, Artificial Sequence
  C-Class oligonucleotides
tcgcgtcgttcggcgcgcgccg

SEQ ID NO: 27 - DNA, 23 bp, Artificial Sequence
  C-Class oligonucleotide
tcgtcgacgttcggcgcgcgccg

SEQ ID NO: 28 - DNA, 21 bp, Artificial Sequence
  C-Class oligonucleotide
tcggacgttcggcgcgcgccg

SEQ ID NO: 29 - DNA, 19 bp, Artificial Sequence
  C-Class oligonucleotide
tcggacgttcggcgcgccg

SEQ ID NO: 30 - DNA, 20 bp, Artificial Sequence
  C-Class oligonucleotide
tcgcgtcgttcggcgcgccg

SEQ ID NO: 31 - DNA, 20 bp, Artificial Sequence
  C-Class oligonucleotide
tcgacgttcggcgcgcgccg

SEQ ID NO: 32 - DNA, 18 bp, Artificial Sequence
  C-Class oligonucleotide
tcgacgttcggcgcgccg

SEQ ID NO: 33 - DNA, 18 bp, Artificial Sequence
  C-Class oligonucleotide
tcgcgtcgttcggcgccg

SEQ ID NO: 34 - DNA, 22 bp, Artificial Sequence
  C-Class oligonucleotide
tcgcgacgttcggcgcgcgccg

SEQ ID NO: 35 - DNA, 22 bp, Artificial Sequence
  C-Class oligonucleotide
tcgtcgttttcggcgcgcgccg

SEQ ID NO: 36 - DNA, 22 bp, Artificial Sequence
  C-Class oligonucleotide
tcgtcgttttcggcggccgccg

SEQ ID NO: 37 - DNA, 24 bp, Artificial Sequence
  C-Class oligonucleotide
tcgtcgttttacggcgccgtgccg

SEQ ID NO: 38 - DNA, 23 bp, Artificial Sequence
  C-Class oligonucleotide
tcgtcgttttcggcgcgcgccgt

SEQ ID NO: 39 - DNA, 23 bp, Artificial Sequence
  P class CpG oligonucleotide
tcgtcgacgatcggcgcgcgccg

SEQ ID NO: 40 - DNA, 23 bp, Artificial Sequence
  P class CpG oligonucleotide
tcgtcgacgatcggcgcgcgccg

SEQ ID NO: 41 - protein, 950 aa, Artificial Sequence
  Enzymatically inactive fragment of SCP
MAKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

SEQ ID NO: 42 - protein, 949 aa, Artificial Sequence
  Enzymatically inactive fragment of SCP
AKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

(19) United States
(12) Patent Application Publication
(10) Pub. No.: US 2022/0387576 A1
(43) Pub. Date: Dec. 8, 2022

(54) IMMUNOGENIC COMPOSITIONS COMPRISING CONJUGATED CAPSULAR SACCHARIDE ANTIGENS AND USES THEREOF

(71) Applicant: Pfizer Inc., New York, NY (US)

(72) Inventors: Annaliesa Sybil Anderson, Upper Saddle River, NJ (US); Caitlyn Gallagher, Ringwood, NJ (US); Jianxin Gu, Paramus, NJ (US); Isis Kanevsky, New York, NY (US); Jin-Hwan Kim, Suffern, NY (US); Justin Keith Moran, West Nyack, NY (US); Suddham Singh, Monroe, NY (US); Naveen Surendran, Dumont, NJ (US)

(21) Appl. No.: 17/824,295

(22) Filed: May 25, 2022

Related U.S. Application Data
(60) Provisional application No. 63/228,761, filed on Aug. 3, 2021.
(60) Provisional application No. 63/194,641, filed on May 28, 2021.

(51) Int. Cl. A61K 39/09; A61P 31/04

(52) U.S. Cl. CPC A61K 39/092; A61P 31/04; A61K 2039/6031

(57) ABSTRACT

The present invention relates to new conjugated capsular saccharide antigens (glycoconjugates), immunogenic compositions comprising said glycoconjugates and uses thereof.

### REFERENCE TO SEQUENCE LISTING

This instant application contains a Sequence Listing which has been submitted electronically in ASCII format and is hereby incorporated by reference in its entirety. Said ASCII copy, created on May 18, 2022, is named PC072611A_ST25.txt and is 25 KB in size.

### FIELD OF THE INVENTION

The present invention relates to new conjugated capsular saccharide antigens (glycoconjugates), immunogenic compositions comprising said glycoconjugates and uses thereof. Immunogenic compositions of the present invention will typically comprise glycoconjugates, wherein the saccharides are derived from serotypes of Streptococcus pneumoniae. The invention also relates to vaccination of human subjects, in particular infants and elderly, against pneumoccocal infections using said glycoconjugates.

### BACKGROUND OF THE INVENTION

Infections caused by pneumococci are a major cause of morbidity and mortality all over the world. Pneumonia, febrile bacteraemia and meningitis are the most common manifestations of invasive pneumococcal disease, whereas bacterial spread within the respiratory tract may result in middle-ear infection, sinusitis or recurrent bronchitis. Compared with invasive disease, the non-invasive manifestations are usually less severe, but considerably more common.

The etiological agent of pneumococcal diseases, Streptococcus pneumoniae (pneumococcus), is a Gram-positive encapsulated coccus, surrounded by a polysaccharide capsule. Differences in the composition of this capsule permit serological differentiation between about 91 capsular types, some of which are frequently associated with pneumococcal disease, others rarely. Invasive pneumococcal infections include pneumonia, meningitis and febrile bacteraemia; among the common non-invasive manifestations are otitis media, sinusitis and bronchitis.

Pneumococcal polysaccharides, in particular capsular polysaccharides, are important immunogens found on the surface of the bacteria. This has led to them being an important component in the design of pneumococcal vaccines. They have proved useful in eliciting immune responses especially when linked to carrier proteins.

Some serotypes, in particular Streptococcus pneumoniae serotype 3, produce large and viscous polysaccharide chains (e.g., for Type 3, chains of glucose/glucuronic acid of 2-3 million Daltons). Its viscosity has made it difficult to handle.

Furthermore, increases in immunogenicity with respect to serotype 3 polysaccharides has been difficult to obtain. For example, in a study of the immunogenicity and safety of an 11-valent pneumococcal protein D conjugate vaccine (11-Pn-PD), no priming effect was observed for serotype 3 in infants who had received three doses of the vaccine followed by a booster dose of either the same vaccine or a pneumococcal polysaccharide vaccine (Nurkka et al. (2004) Ped. Inf. Dis. J., 23:1008-1014). In another study, opsonophagocytic assay (OPA) results from infants who had received doses of 11-Pn-PD failed to show antibody responses for serotype 3 at levels comparable to other tested serotypes (Gatchalian et al., 17th annual Meeting of the Eur. Soc. Paed. Inf. Dis. (ESPID), Poster No. 4, PIA Poster Session 1, Istanbul Turkey, Mar. 27, 2001). In yet another study, which assessed the efficacy of an 11-Pn-PD in the prevention of acute otitis media, the vaccine did not provide protection against episodes caused by serotype 3 (Prymula et al. The Lancet, Vol. 367: 740-748 (Mar. 4, 2006)).

Thus, there is a need for antigens which are able to generate a more robust immune response to Streptococcus pneumoniae serotype 3.

The present invention provides in particular Streptococcus pneumoniae serotype 3 glycoconjugates which show improved immunogenicity. The present invention also provides processes which generate Streptococcus pneumoniae serotype 3 glycoconjugates with fewer operational steps, and better conjugation yields.

### SUMMARY OF THE INVENTION

In an aspect the invention relates to a method of making a Streptococcus pneumoniae serotype 3 glycoconjugate, comprising the steps of:

(a) reacting an isolated Streptococcus pneumoniae serotype 3 capsular polysaccharide with an oxidizing agent;
(b) compounding the activated polysaccharide of step (a) with a carrier protein; and
(c) reacting the compounded activated polysaccharide and carrier protein with a reducing agent to form a glycoconjugate, wherein the isolated polysaccharide is sized before the activation step (a) to a weight average molecular weight between 100 kDa and 200 kDa and wherein the reduction reaction (c) is carried out in aprotic solvent.

In an aspect the oxidizing agent is periodate or periodic acid.

In an aspect, the degree of oxidation of the activated serotype 3 polysaccharide is between 11 to 19 or about 15.

In an aspect, the reduction reaction (c) is carried out in a solution consisting essentially of dimethylsulphoxide (DMSO).

The invention further relates to a Streptococcus pneumoniae serotype 3 glycoconjugate produced according to these methods.

In an aspect, the glycoconjugate comprises a serotype 3 capsular polysaccharide wherein the weight average molecular weight (Mw) of said polysaccharide before conjugation is between 120 kDa and 180 kDa or about 150 kDa.

In an aspect, the carrier protein is CRM₁₉₇ or SCP.

The invention further relates to an immunogenic composition comprising said Streptococcus pneumoniae serotype 3 glycoconjugate.

The invention further relates to a method of making a Streptococcus pneumoniae serotype 3 glycoconjugate, comprising the steps of:

(a) reacting an isolated Streptococcus pneumoniae serotype 3 capsular polysaccharide with 1,1′-carbonyldiimidazole (CDI) or 1,1′-Carbonyl-di-(1,2,4-triazole) (CDT) in an aprotic solvent;
(b) reacting the activated polysaccharide of step (a) with a carrier protein in an aprotic solvent to form a glycoconjugate.

In an aspect, the isolated polysaccharide is sized to a weight average molecular weight between 100 kDa and 200 kDa.

In an aspect, the activating reaction a) is carried out in a solution consisting essentially of dimethylsulphoxide (DMSO) or dimethylformamide (DMF).

In an aspect, the conjugation reaction b) is carried out in a solution consisting essentially of dimethylsulphoxide (DMSO) or dimethylformamide (DMF).

In an aspect, a weak organic base is added to the reaction mixture after the activating reaction a) but before the conjugation reaction b).

### FIGURES

FIG. 1 shows a repeating polysaccharide structure of the S. pneumoniae serotype 3 capsular polysaccharide.

FIG. 2 shows a general scheme for the preparation of Streptococcus pneumoniae serotype 3 glycoconjugate of the invention using CDI or CDT chemistry. Pn3 poly=S. pneumoniae serotype 3 capsular polysaccharide; CP=Carrier Protein, CDI=1,1′-carbonyldiimidazole; CDT=1,1′-Carbonyl-di-(1,2,4-triazole).

FIG. 3 shows opsonophagocytic activity (OPA) titers for Serotype 3-CRM₁₉₇ conjugates in mice comprising polysaccharide of different size. Sized Serotype 3 polysaccharides (˜25, 150, or 250 kDa) conjugated to CRM₁₉₇ using either RAC/Aqueous or RAC/DMSO conjugation was used to vaccinate mice.

FIG. 4 shows opsonophagocytic activity (OPA) titers for Serotype 3-CRM₁₉₇ conjugates in mice with different Degree of Activation (DoA). Sized Serotype 3 polysaccharides conjugated to CRM₁₉₇ using the either RAC/Aqueous or RAC/DMSO conjugation were used to vaccinate mice.

FIG. 5 shows the opsonophagocytic activity (OPA) titers for Serotype 3 conjugates to -CRM₁₉₇, -SCP, or Tetanus toxoid (TT) in mice. Reductive Amination in DMSO (RAC/DMSO) was used.

FIG. 6 shows opsonophagocytic activity (OPA) titers for Serotype 3-CRM₁₉₇ conjugates in mice. Different chemistries have been used (Reductive Amination in aqueous (RAC/Aq.), Reductive Amination in DMSO (RAC/DMSO), eTEC linked glycoconjugates (eTEC) or CDI chemistry (CDI Direct).

FIG. 7A-7B show immune response (OPA and IgG) to RAC/DMSO carriers in infant rhesus macaques. FIG. 7A: Opsonophagocytic titers measured from sera collected at 4 week post each vaccination time point between different carriers on RAC/DMSO chemistry. FIG. 7B: IgG titers measured from sera collected at 4 week post each vaccination time point between different carriers on RAC/DMSO chemistry. Each dot represents individual animal and data expressed as geomean titers with 95% confidence interval. Statistical significance (p values) determined based on one-way ANOVA. PD—post dose, LLOQ—lower limit of quantitation.

FIG. 8A-8B show immune response (OPA and IgG) to RAC/aqueous carriers in infant rhesus macaques. FIG. 8A: Opsonophagocytic titers measured from sera collected at 4 week post each vaccination time point between different poly size on RAC/Aq chemistry. FIG. 8B: IgG titers measured from sera collected at 4 week post each vaccination time point between different polysize on RAC/Aq chemistry. Each dot represents individual animal and data expressed as geomean titers with 95% confidence interval. Statistical significance (p values) determined based on one-way ANOVA. PD—post dose, LLOQ—lower limit of quantitation.

1. Glycoconjugates of the Invention

The present invention is directed in part to conjugated capsular saccharide antigens (also named glycoconjugates), where saccharides are derived from serotypes of S. pneumoniae, in particular from serotype 3. For the purpose of the invention the term ‘glycoconjugate’ indicates a capsular saccharide linked covalently to a carrier protein. In one embodiment a capsular saccharide is linked directly to a carrier protein. In a second embodiment a bacterial saccharide is linked to a protein through a spacer/linker.

1.1 Pneumococcal Saccharide from S. pneumoniae Serotype 3

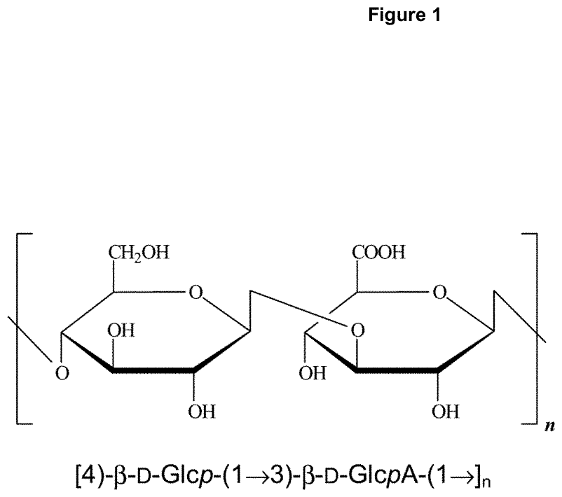

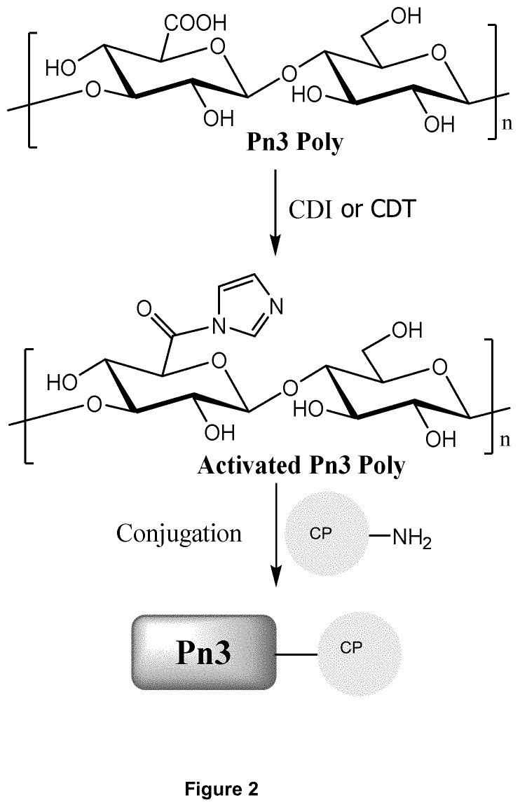

As shown at FIG. 1, the polysaccharide repeating unit of serotype 3 consists of a linear disaccharide unit with one glucopyranose (Glcp) and one glucuronic acid (GlcpA) (see e.g. Geno K et al. (2015) Clin Microbiol Rev Vol 28:3, p 871-899).

The source of bacterial polysaccharide according to this invention can be Streptococcus pneumoniae serotype 3 bacterial cells. Bacterial strains which can be used as source of Streptococcus pneumoniae serotype 3 polysaccharides may be obtained from established culture collections (such as for example from the Streptococcal Reference Laboratory (Centers for Disease Control and Prevention, Atlanta, Ga. USA)) or clinical specimens.

Serotype 3 polysaccharides can be obtained directly from bacteria using isolation procedures known to one of ordinary skill in the art (see for example methods disclosed in US2006/0228380, US2006/0228381, US2007/0184071, US2007/0184072, US2007/0231340, and US2008/0102498 and WO2008/118752). They can also be produced using synthetic protocols known to the man skilled in the art. They can also be purchased (such as for example from the American Type Culture Collection (ATCC, Manassas, Va. USA) (e.g., reference No. ATCC 172-X or ATCC 33-X)).

In case the serotype 3 polysaccharide is obtained directly from bacteria, the bacterial cells can be grown in a medium, preferably in a soy based medium. Following fermentation of bacterial cells that produce S. pneumoniae serotype 3 capsular polysaccharides, the bacterial cells can be lysed to produce a cell lysate. The serotype 3 polysaccharide may then be isolated from the cell lysate using purification techniques known in the art, including the use of centrifugation, depth filtration, precipitation, ultrafiltration, treatment with activate carbon, diafiltration and/or column chromatography (see, for example, US2006/0228380, US2006/0228381 and WO2008/118752). The purified serotype 3 capsular polysaccharide can then be used for the preparation of immunogenic conjugates.

The isolated serotype 3 capsular polysaccharide obtained by purification of serotype 3 polysaccharide from the S. pneumoniae lysate and optionally sizing of the purified polysaccharide can be characterized by different parameters including, for example the weight average molecular weight (Mw).

The molecular weight of the polysaccharide can be measured by Size Exclusion Chromatography (SEC) combined with Multiangle Laser Light Scattering detector (MALLS).

In a preferred embodiment, the isolated serotype 3 capsular polysaccharide (i.e. purified before further treatment) has a weight average molecular weight between 5 kDa and 5000 kDa. In an embodiment, the isolated serotype 3 capsular polysaccharide has a weight average molecular weight between 5 kDa and 4000 kDa. In an embodiment, the isolated serotype 3 capsular polysaccharide has a weight average molecular weight between 5 kDa and 3000 kDa. In an embodiment, the isolated serotype 3 capsular polysaccharide has a weight average molecular weight between 5 kDa and 2000 kDa. In an embodiment, the isolated serotype 3 capsular polysaccharide has a weight average molecular weight between 5 kDa and 1500 kDa. In an embodiment, the isolated serotype 3 capsular polysaccharide has a weight average molecular weight between 5 kDa and 1000 kDa. In an embodiment, the isolated serotype 3 capsular polysaccharide has a weight average molecular weight between 5 kDa and 500 kDa. In an embodiment, the isolated serotype 3 capsular polysaccharide has a weight average molecular weight between 5 kDa and 400 kDa. In an embodiment, the isolated serotype 3 capsular polysaccharide has a weight average molecular weight between 5 kDa and 300 kDa. In an embodiment, the isolated serotype 3 capsular polysaccharide has a weight average molecular weight between 5 kDa and 200 kDa. In an embodiment, the isolated serotype 3 capsular polysaccharide has a weight average molecular weight between 5 kDa and 100 kDa.

In an embodiment, the isolated serotype 3 capsular polysaccharide has a weight average molecular weight between 50 kDa and 5000 kDa. In an embodiment, the isolated serotype 3 capsular polysaccharide has a weight average molecular weight between 50 kDa and 4000 kDa. In an embodiment, the isolated serotype 3 capsular polysaccharide has a weight average molecular weight between 50 kDa and 3000 kDa. In an embodiment, the isolated serotype 3 capsular polysaccharide has a weight average molecular weight between 50 kDa and 2000 kDa. In an embodiment, the isolated serotype 3 capsular polysaccharide has a weight average molecular weight between 50 kDa and 1500 kDa. In an embodiment, the isolated serotype 3 capsular polysaccharide has a weight average molecular weight between 50 kDa and 1000 kDa. In an embodiment, the isolated serotype 3 capsular polysaccharide has a weight average molecular weight between 50 kDa and 500 kDa. In an embodiment, the isolated serotype 3 capsular polysaccharide has a weight average molecular weight between 50 kDa and 400 kDa. In an embodiment, the isolated serotype 3 capsular polysaccharide has a weight average molecular weight between 50 kDa and 300 kDa. In an embodiment, the isolated serotype 3 capsular polysaccharide has a weight average molecular weight between 50 kDa and 200 kDa. In an embodiment, the isolated serotype 3 capsular polysaccharide has a weight average molecular weight between 50 kDa and 100 kDa.

In an embodiment, the isolated serotype 3 capsular polysaccharide has a weight average molecular weight between 100 kDa and 5000 kDa. In an embodiment, the isolated serotype 3 capsular polysaccharide has a weight average molecular weight between 100 kDa and 4000 kDa. In an embodiment, the isolated serotype 3 capsular polysaccharide has a weight average molecular weight between 100 kDa and 3000 kDa. In an embodiment, the isolated serotype 3 capsular polysaccharide has a weight average molecular weight between 100 kDa and 2000 kDa. In an embodiment, the isolated serotype 3 capsular polysaccharide has a weight average molecular weight between 100 kDa and 1500 kDa. In an embodiment, the isolated serotype 3 capsular polysaccharide has a weight average molecular weight between 100 kDa and 1000 kDa. In an embodiment, the isolated serotype 3 capsular polysaccharide has a weight average molecular weight between 100 kDa and 500 kDa. In an embodiment, the isolated serotype 3 capsular polysaccharide has a weight average molecular weight between 100 kDa and 400 kDa. In an embodiment, the isolated serotype 3 capsular polysaccharide has a weight average molecular weight between 100 kDa and 300 kDa. In an embodiment, the isolated serotype 3 capsular polysaccharide has a weight average molecular weight between 100 kDa and 200 kDa.

In an embodiment, the isolated serotype 3 capsular polysaccharide has a weight average molecular weight between 150 kDa and 5000 kDa. In an embodiment, the isolated serotype 3 capsular polysaccharide has a weight average molecular weight between 150 kDa and 4000 kDa. In an embodiment, the isolated serotype 3 capsular polysaccharide has a weight average molecular weight between 150 kDa and 3000 kDa. In an embodiment, the isolated serotype 3 capsular polysaccharide has a weight average molecular weight between 150 kDa and 2000 kDa. In an embodiment, the isolated serotype 3 capsular polysaccharide has a weight average molecular weight between 150 kDa and 1500 kDa. In an embodiment, the isolated serotype 3 capsular polysaccharide has a weight average molecular weight between 150 kDa and 1000 kDa. In an embodiment, the isolated serotype 3 capsular polysaccharide has a weight average molecular weight between 150 kDa and 500 kDa. In an embodiment, the isolated serotype 3 capsular polysaccharide has a weight average molecular weight between 150 kDa and 400 kDa. In an embodiment, the isolated serotype 3 capsular polysaccharide has a weight average molecular weight between 150 kDa and 300 kDa. In an embodiment, the isolated serotype 3 capsular polysaccharide has a weight average molecular weight between 150 kDa and 200 kDa.

In an embodiment, the isolated serotype 3 capsular polysaccharide has a weight average molecular weight between 200 kDa and 5000 kDa. In an embodiment, the isolated serotype 3 capsular polysaccharide has a weight average molecular weight between 200 kDa and 4000 kDa. In an embodiment, the isolated serotype 3 capsular polysaccharide has a weight average molecular weight between 200 kDa and 3000 kDa. In an embodiment, the isolated serotype 3 capsular polysaccharide has a weight average molecular weight between 200 kDa and 2000 kDa. In an embodiment, the isolated serotype 3 capsular polysaccharide has a weight average molecular weight between 200 kDa and 1500 kDa. In an embodiment, the isolated serotype 3 capsular polysaccharide has a weight average molecular weight between 200 kDa and 1000 kDa. In an embodiment, the isolated serotype 3 capsular polysaccharide has a weight average molecular weight between 200 kDa and 500 kDa. In an embodiment, the isolated serotype 3 capsular polysaccharide has a weight average molecular weight between 200 kDa and 400 kDa. In an embodiment, the isolated serotype 3 capsular polysaccharide has a weight average molecular weight between 200 kDa and 300 kDa.

In an embodiment, the isolated serotype 3 capsular polysaccharide has a weight average molecular weight between 300 kDa and 5000 kDa. In an embodiment, the isolated serotype 3 capsular polysaccharide has a weight average molecular weight between 300 kDa and 4000 kDa. In an embodiment, the isolated serotype 3 capsular polysaccharide has a weight average molecular weight between 300 kDa and 3000 kDa. In an embodiment, the isolated serotype 3 capsular polysaccharide has a weight average molecular weight between 300 kDa and 2000 kDa. In an embodiment, the isolated serotype 3 capsular polysaccharide has a weight average molecular weight between 300 kDa and 1500 kDa. In an embodiment, the isolated serotype 3 capsular polysaccharide has a weight average molecular weight between 300 kDa and 1000 kDa. In an embodiment, the isolated serotype 3 capsular polysaccharide has a weight average molecular weight between 300 kDa and 500 kDa. In an embodiment, the isolated serotype 3 capsular polysaccharide has a weight average molecular weight between 300 kDa and 400 kDa.

In an embodiment, the isolated serotype 3 capsular polysaccharide has a weight average molecular weight between 400 kDa and 5000 kDa. In an embodiment, the isolated serotype 3 capsular polysaccharide has a weight average molecular weight between 400 kDa and 4000 kDa. In an embodiment, the isolated serotype 3 capsular polysaccharide has a weight average molecular weight between 400 kDa and 3000 kDa. In an embodiment, the isolated serotype 3 capsular polysaccharide has a weight average molecular weight between 400 kDa and 2000 kDa. In an embodiment, the isolated serotype 3 capsular polysaccharide has a weight average molecular weight between 400 kDa and 1500 kDa. In an embodiment, the isolated serotype 3 capsular polysaccharide has a weight average molecular weight between 400 kDa and 1000 kDa. In an embodiment, the isolated serotype 3 capsular polysaccharide has a weight average molecular weight between 400 kDa and 500 kDa.

In an embodiment, the isolated serotype 3 capsular polysaccharide has a weight average molecular weight between 500 kDa and 5000 kDa. In an embodiment, the isolated serotype 3 capsular polysaccharide has a weight average molecular weight between 500 kDa and 4000 kDa. In an embodiment, the isolated serotype 3 capsular polysaccharide has a weight average molecular weight between 500 kDa and 3000 kDa. In an embodiment, the isolated serotype 3 capsular polysaccharide has a weight average molecular weight between 500 kDa and 2000 kDa. In an embodiment, the isolated serotype 3 capsular polysaccharide has a weight average molecular weight between 500 kDa and 1500 kDa. In an embodiment, the isolated serotype 3 capsular polysaccharide has a weight average molecular weight between 500 kDa and 1000 kDa.

In an embodiment, the isolated serotype 3 capsular polysaccharide has a weight average molecular weight between 750 kDa and 5000 kDa. In an embodiment, the isolated serotype 3 capsular polysaccharide has a weight average molecular weight between 750 kDa and 4000 kDa. In an embodiment, the isolated serotype 3 capsular polysaccharide has a weight average molecular weight between 750 kDa and 3000 kDa. In an embodiment, the isolated serotype 3 capsular polysaccharide has a weight average molecular weight between 750 kDa and 2000 kDa. In an embodiment, the isolated serotype 3 capsular polysaccharide has a weight average molecular weight between 750 kDa and 1500 kDa. In an embodiment, the isolated serotype 3 capsular polysaccharide has a weight average molecular weight between 750 kDa and 1000 kDa.

In an embodiment, the isolated serotype 3 capsular polysaccharide has a weight average molecular weight between 1000 kDa and 5000 kDa. In an embodiment, the isolated serotype 3 capsular polysaccharide has a weight average molecular weight between 1000 kDa and 4000 kDa. In an embodiment, the isolated serotype 3 capsular polysaccharide has a weight average molecular weight between 1000 kDa and 3000 kDa. In an embodiment, the isolated serotype 3 capsular polysaccharide has a weight average molecular weight between 1000 kDa and 2000 kDa. In an embodiment, the isolated serotype 3 capsular polysaccharide has a weight average molecular weight between 1000 kDa and 1500 kDa.

In an embodiment, the isolated serotype 3 capsular polysaccharide has a weight average molecular weight between 1500 kDa and 5000 kDa. In an embodiment, the isolated serotype 3 capsular polysaccharide has a weight average molecular weight between 1500 kDa and 4000 kDa. In an embodiment, the isolated serotype 3 capsular polysaccharide has a weight average molecular weight between 1500 kDa and 3000 kDa. In an embodiment, the isolated serotype 3 capsular polysaccharide has a weight average molecular weight between 1500 kDa and 2000 kDa.

In an embodiment, the isolated serotype 3 capsular polysaccharide has a weight average molecular weight between 2000 kDa and 5000 kDa. In an embodiment, the isolated serotype 3 capsular polysaccharide has a weight average molecular weight between 2000 kDa and 4000 kDa. In an embodiment, the isolated serotype 3 capsular polysaccharide has a weight average molecular weight between 2000 kDa and 3000 kDa.

In an embodiment, the isolated serotype 3 capsular polysaccharide has a weight average molecular weight between 2500 kDa and 5000 kDa. In an embodiment, the isolated serotype 3 capsular polysaccharide has a weight average molecular weight between 2500 kDa and 4000 kDa. In an embodiment, the isolated serotype 3 capsular polysaccharide has a weight average molecular weight between 2500 kDa and 3000 kDa.

Any whole number integer within any of the above ranges is contemplated as an embodiment of the disclosure.

Preferably, in order to generate serotype 3 conjugates with advantageous filterability characteristics, immunogenicity and/or yields, sizing of the polysaccharide to a target molecular weight range is performed prior to the conjugation to a carrier protein. Advantageously, the size of the purified serotype 3 polysaccharide is reduced while preserving critical features of the structure of the polysaccharide. Mechanical or chemical sizing maybe employed. In an embodiment, the size of the purified serotype 3 polysaccharide is reduced by chemical hydrolysis. Chemical hydrolysis maybe conducted using a mild acid (e.g. acetic acid, formic acid or propanoic acid). Chemical hydrolysis may also be conducted using a diluted strong acid (such as diluted hydrochloric acid, diluted sulfuric acid, diluted phosphoric acid, diluted nitric acid or diluted perchloric acid). In a preferred embodiment, chemical hydrolysis is conducted using acetic acid. The size of the purified serotype 3 polysaccharide can also be reduced by mechanical homogenization. In an embodiment, the size of the purified serotype 3 polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

The high-pressure homogenization process is particularly appropriate for reducing the size of the purified serotype 3 polysaccharide while preserving the structural features of the polysaccharide.

In a preferred embodiment, the isolated serotype 3 capsular polysaccharide is sized to a weight average molecular weight between 5 kDa and 1000 kDa. In an embodiment, the isolated serotype 3 capsular polysaccharide is sized to a weight average molecular weight between 5 kDa and 900 kDa. In an embodiment, the isolated serotype 3 capsular polysaccharide is sized to a weight average molecular weight between 5 kDa and 800 kDa. In an embodiment, the isolated serotype 3 capsular polysaccharide is sized to a weight average molecular weight between 5 kDa and 700 kDa. In an embodiment, the isolated serotype 3 capsular polysaccharide is sized to a weight average molecular weight between 5 kDa and 600 kDa. In an embodiment, the isolated serotype 3 capsular polysaccharide is sized to a weight average molecular weight between 5 kDa and 500 kDa. In an embodiment, the isolated serotype 3 capsular polysaccharide is sized to a weight average molecular weight between 5 kDa and 450 kDa. In an embodiment, the isolated serotype 3 capsular polysaccharide is sized to a weight average molecular weight between 5 kDa and 400 kDa. In an embodiment, the isolated serotype 3 capsular polysaccharide is sized to a weight average molecular weight between 5 kDa and 350 kDa. In an embodiment, the isolated serotype 3 capsular polysaccharide is sized to a weight average molecular weight between 5 kDa and 300 kDa. In an embodiment, the isolated serotype 3 capsular polysaccharide is sized to a weight average molecular weight between 5 kDa and 250 kDa. In an embodiment, the isolated serotype 3 capsular polysaccharide is sized to a weight average molecular weight between 5 kDa and 200 kDa. In an embodiment, the isolated serotype 3 capsular polysaccharide is sized to a weight average molecular weight between 5 kDa and 150 kDa. In an embodiment, the isolated serotype 3 capsular polysaccharide is sized to a weight average molecular weight between 5 kDa and 100 kDa. In an embodiment, the isolated serotype 3 capsular polysaccharide is sized to a weight average molecular weight between 5 kDa and 50 kDa.

In an embodiment, the serotype 3 glycoconjugate of the present invention comprises a serotype 3 capsular polysaccharide wherein the weight average molecular weight (Mw) of said polysaccharide before conjugation is between 25 kDa and 1,000 kDa. In an embodiment, the weight average molecular weight (Mw) is between 25 kDa and 750 kDa. In an embodiment, the weight average molecular weight (Mw) is between 25 kDa and 700 kDa. In an embodiment, the weight average molecular weight (Mw) is between 25 kDa and 600 kDa. In an embodiment, the weight average molecular weight (Mw) is between 25 kDa and 500 kDa. In an embodiment, the weight average molecular weight (Mw) is between 25 kDa and 400 kDa. In an embodiment, the weight average molecular weight (Mw) is between 25 kDa and 300 kDa. In an embodiment, the weight average molecular weight (Mw) is between 25 kDa and 200 kDa. In an embodiment, the weight average molecular weight (Mw) is between 25 kDa and 175 kDa. In an embodiment, the weight average molecular weight (Mw) is between 25 kDa and 150 kDa. In an embodiment, the weight average molecular weight (Mw) is between 25 kDa and 140 kDa. In an embodiment, the weight average molecular weight (Mw) is between 25 kDa and 130 kDa. In an embodiment, the weight average molecular weight (Mw) is between 25 kDa and 120 kDa. In an embodiment, the weight average molecular weight (Mw) is between 25 kDa and 110 kDa.

In an embodiment, the isolated serotype 3 capsular polysaccharide is sized to a weight average molecular weight between 50 kDa and 1000 kDa. In an embodiment, the isolated serotype 3 capsular polysaccharide is sized to a weight average molecular weight between 50 kDa and 900 kDa. In an embodiment, the isolated serotype 3 capsular polysaccharide is sized to a weight average molecular weight between 50 kDa and 800 kDa. In an embodiment, the isolated serotype 3 capsular polysaccharide is sized to a weight average molecular weight between 50 kDa and 700 kDa. In an embodiment, the isolated serotype 3 capsular polysaccharide is sized to a weight average molecular weight between 50 kDa and 600 kDa. In an embodiment, the isolated serotype 3 capsular polysaccharide is sized to a weight average molecular weight between 50 kDa and 500 kDa. In an embodiment, the isolated serotype 3 capsular polysaccharide is sized to a weight average molecular weight between 50 kDa and 450 kDa. In an embodiment, the isolated serotype 3 capsular polysaccharide is sized to a weight average molecular weight between 50 kDa and 400 kDa. In an embodiment, the isolated serotype 3 capsular polysaccharide is sized to a weight average molecular weight between 50 kDa and 350 kDa. In an embodiment, the isolated serotype 3 capsular polysaccharide is sized to a weight average molecular weight between 50 kDa and 300 kDa. In an embodiment, the isolated serotype 3 capsular polysaccharide is sized to a weight average molecular weight between 50 kDa and 250 kDa. In an embodiment, the isolated serotype 3 capsular polysaccharide is sized to a weight average molecular weight between 50 kDa and 200 kDa. In an embodiment, the isolated serotype 3 capsular polysaccharide is sized to a weight average molecular weight between 50 kDa and 175 kDa. In an embodiment, the isolated serotype 3 capsular polysaccharide is sized to a weight average molecular weight between 50 kDa and 150 kDa. In an embodiment, the isolated serotype 3 capsular polysaccharide is sized to a weight average molecular weight between 50 kDa and 100 kDa.

In an embodiment, the isolated serotype 3 capsular polysaccharide is sized to a weight average molecular weight between 100 kDa and 1000 kDa. In an embodiment, the isolated serotype 3 capsular polysaccharide is sized to a weight average molecular weight between 100 kDa and 900 kDa. In an embodiment, the isolated serotype 3 capsular polysaccharide is sized to a weight average molecular weight between 100 kDa and 800 kDa. In an embodiment, the isolated serotype 3 capsular polysaccharide is sized to a weight average molecular weight between 100 kDa and 700 kDa. In an embodiment, the isolated serotype 3 capsular polysaccharide is sized to a weight average molecular weight between 100 kDa and 600 kDa. In an embodiment, the isolated serotype 3 capsular polysaccharide is sized to a weight average molecular weight between 100 kDa and 500 kDa. In an embodiment, the isolated serotype 3 capsular polysaccharide is sized to a weight average molecular weight between 100 kDa and 450 kDa. In an embodiment, the isolated serotype 3 capsular polysaccharide is sized to a weight average molecular weight between 100 kDa and 400 kDa. In an embodiment, the isolated serotype 3 capsular polysaccharide is sized to a weight average molecular weight between 100 kDa and 350 kDa. In an embodiment, the isolated serotype 3 capsular polysaccharide is sized to a weight average molecular weight between 100 kDa and 300 kDa. In an embodiment, the isolated serotype 3 capsular polysaccharide is sized to a weight average molecular weight between 100 kDa and 250 kDa. In an embodiment, the isolated serotype 3 capsular polysaccharide is sized to a weight average molecular weight between 100 kDa and 200 kDa. In an embodiment, the isolated serotype 3 capsular polysaccharide is sized to a weight average molecular weight between 100 kDa and 175 kDa. In an embodiment, the isolated serotype 3 capsular polysaccharide is sized to a weight average molecular weight between 100 kDa and 150 kDa.

In an embodiment, the isolated serotype 3 capsular polysaccharide is sized to a weight average molecular weight between 110 kDa and 150 kDa. In an embodiment, the isolated serotype 3 capsular polysaccharide is sized to a weight average molecular weight between 120 kDa and 150 kDa. In an embodiment, the isolated serotype 3 capsular polysaccharide is sized to a weight average molecular weight between 110 kDa and 150 kDa. In an embodiment, the isolated serotype 3 capsular polysaccharide is sized to a weight average molecular weight between 130 kDa and 150 kDa. In an embodiment, the isolated serotype 3 capsular polysaccharide is sized to a weight average molecular weight between 120 kDa and 150 kDa. In an embodiment, the isolated serotype 3 capsular polysaccharide is sized to a weight average molecular weight between 120 kDa and 140 kDa. In an embodiment, the isolated serotype 3 capsular polysaccharide is sized to a weight average molecular weight between 120 kDa and 130 kDa. In an embodiment, the isolated serotype 3 capsular polysaccharide is sized to a weight average molecular weight between 130 kDa and 150 kDa. In an embodiment, the isolated serotype 3 capsular polysaccharide is sized to a weight average molecular weight between 130 kDa and 140 kDa.

In an embodiment, the isolated serotype 3 capsular polysaccharide is sized to a weight average molecular weight between 150 kDa and 1000 kDa. In an embodiment, the isolated serotype 3 capsular polysaccharide is sized to a weight average molecular weight between 150 kDa and 900 kDa. In an embodiment, the isolated serotype 3 capsular polysaccharide is sized to a weight average molecular weight between 150 kDa and 800 kDa. In an embodiment, the isolated serotype 3 capsular polysaccharide is sized to a weight average molecular weight between 150 kDa and 700 kDa. In an embodiment, the isolated serotype 3 capsular polysaccharide is sized to a weight average molecular weight between 150 kDa and 600 kDa. In an embodiment, the isolated serotype 3 capsular polysaccharide is sized to a weight average molecular weight between 150 kDa and 500 kDa. In an embodiment, the isolated serotype 3 capsular polysaccharide is sized to a weight average molecular weight between 150 kDa and 450 kDa. In an embodiment, the isolated serotype 3 capsular polysaccharide is sized to a weight average molecular weight between 150 kDa and 400 kDa. In an embodiment, the isolated serotype 3 capsular polysaccharide is sized to a weight average molecular weight between 150 kDa and 350 kDa. In an embodiment, the isolated serotype 3 capsular polysaccharide is sized to a weight average molecular weight between 150 kDa and 300 kDa. In an embodiment, the isolated serotype 3 capsular polysaccharide is sized to a weight average molecular weight between 150 kDa and 250 kDa. In an embodiment, the isolated serotype 3 capsular polysaccharide is sized to a weight average molecular weight between 150 kDa and 200 kDa. In an embodiment, the isolated serotype 3 capsular polysaccharide is sized to a weight average molecular weight between 150 kDa and 175 kDa.

In an embodiment, the isolated serotype 3 capsular polysaccharide is sized to a weight average molecular weight between 200 kDa and 1000 kDa. In an embodiment, the isolated serotype 3 capsular polysaccharide is sized to a weight average molecular weight between 200 kDa and 900 kDa. In an embodiment, the isolated serotype 3 capsular polysaccharide is sized to a weight average molecular weight between 200 kDa and 800 kDa. In an embodiment, the isolated serotype 3 capsular polysaccharide is sized to a weight average molecular weight between 200 kDa and 700 kDa. In an embodiment, the isolated serotype 3 capsular polysaccharide is sized to a weight average molecular weight between 200 kDa and 600 kDa. In an embodiment, the isolated serotype 3 capsular polysaccharide is sized to a weight average molecular weight between 200 kDa and 500 kDa. In an embodiment, the isolated serotype 3 capsular polysaccharide is sized to a weight average molecular weight between 200 kDa and 450 kDa. In an embodiment, the isolated serotype 3 capsular polysaccharide is sized to a weight average molecular weight between 200 kDa and 400 kDa. In an embodiment, the isolated serotype 3 capsular polysaccharide is sized to a weight average molecular weight between 200 kDa and 350 kDa. In an embodiment, the isolated serotype 3 capsular polysaccharide is sized to a weight average molecular weight between 200 kDa and 300 kDa. In an embodiment, the isolated serotype 3 capsular polysaccharide is sized to a weight average molecular weight between 200 kDa and 250 kDa.

In an embodiment, the isolated serotype 3 capsular polysaccharide is sized to a weight average molecular weight between 250 kDa and 1000 kDa. In an embodiment, the isolated serotype 3 capsular polysaccharide is sized to a weight average molecular weight between 250 kDa and 900 kDa. In an embodiment, the isolated serotype 3 capsular polysaccharide is sized to a weight average molecular weight between 250 kDa and 800 kDa. In an embodiment, the isolated serotype 3 capsular polysaccharide is sized to a weight average molecular weight between 250 kDa and 700 kDa. In an embodiment, the isolated serotype 3 capsular polysaccharide is sized to a weight average molecular weight between 250 kDa and 600 kDa. In an embodiment, the isolated serotype 3 capsular polysaccharide is sized to a weight average molecular weight between 250 kDa and 500 kDa. In an embodiment, the isolated serotype 3 capsular polysaccharide is sized to a weight average molecular weight between 250 kDa and 450 kDa. In an embodiment, the isolated serotype 3 capsular polysaccharide is sized to a weight average molecular weight between 250 kDa and 400 kDa. In an embodiment, the isolated serotype 3 capsular polysaccharide is sized to a weight average molecular weight between 250 kDa and 350 kDa. In an embodiment, the isolated serotype 3 capsular polysaccharide is sized to a weight average molecular weight between 250 kDa and 300 kDa.

In an embodiment, the isolated serotype 3 capsular polysaccharide is sized to a weight average molecular weight between 300 kDa and 1000 kDa. In an embodiment, the isolated serotype 3 capsular polysaccharide is sized to a weight average molecular weight between 300 kDa and 900 kDa. In an embodiment, the isolated serotype 3 capsular polysaccharide is sized to a weight average molecular weight between 300 kDa and 800 kDa. In an embodiment, the isolated serotype 3 capsular polysaccharide is sized to a weight average molecular weight between 300 kDa and 700 kDa. In an embodiment, the isolated serotype 3 capsular polysaccharide is sized to a weight average molecular weight between 300 kDa and 600 kDa. In an embodiment, the isolated serotype 3 capsular polysaccharide is sized to a weight average molecular weight between 300 kDa and 500 kDa. In an embodiment, the isolated serotype 3 capsular polysaccharide is sized to a weight average molecular weight between 300 kDa and 450 kDa. In an embodiment, the isolated serotype 3 capsular polysaccharide is sized to a weight average molecular weight between 300 kDa and 400 kDa. In an embodiment, the isolated serotype 3 capsular polysaccharide is sized to a weight average molecular weight between 300 kDa and 350 kDa.

In an embodiment, the isolated serotype 3 capsular polysaccharide is sized to a weight average molecular weight between 350 kDa and 1000 kDa. In an embodiment, the isolated serotype 3 capsular polysaccharide is sized to a weight average molecular weight between 350 kDa and 900 kDa. In an embodiment, the isolated serotype 3 capsular polysaccharide is sized to a weight average molecular weight between 350 kDa and 800 kDa. In an embodiment, the isolated serotype 3 capsular polysaccharide is sized to a weight average molecular weight between 350 kDa and 700 kDa. In an embodiment, the isolated serotype 3 capsular polysaccharide is sized to a weight average molecular weight between 350 kDa and 600 kDa. In an embodiment, the isolated serotype 3 capsular polysaccharide is sized to a weight average molecular weight between 350 kDa and 500 kDa. In an embodiment, the isolated serotype 3 capsular polysaccharide is sized to a weight average molecular weight between 350 kDa and 450 kDa. In an embodiment, the isolated serotype 3 capsular polysaccharide is sized to a weight average molecular weight between 350 kDa and 400 kDa.

In an embodiment, the isolated serotype 3 capsular polysaccharide is sized to a weight average molecular weight between 400 kDa and 1000 kDa. In an embodiment, the isolated serotype 3 capsular polysaccharide is sized to a weight average molecular weight between 400 kDa and 900 kDa. In an embodiment, the isolated serotype 3 capsular polysaccharide is sized to a weight average molecular weight between 400 kDa and 800 kDa. In an embodiment, the isolated serotype 3 capsular polysaccharide is sized to a weight average molecular weight between 400 kDa and 700 kDa. In an embodiment, the isolated serotype 3 capsular polysaccharide is sized to a weight average molecular weight between 400 kDa and 600 kDa. In an embodiment, the isolated serotype 3 capsular polysaccharide is sized to a weight average molecular weight between 400 kDa and 500 kDa. In an embodiment, the isolated serotype 3 capsular polysaccharide is sized to a weight average molecular weight between 400 kDa and 450 kDa.

In an embodiment, the isolated serotype 3 capsular polysaccharide is sized to a weight average molecular weight between 450 kDa and 1000 kDa. In an embodiment, the isolated serotype 3 capsular polysaccharide is sized to a weight average molecular weight between 450 kDa and 900 kDa. In an embodiment, the isolated serotype 3 capsular polysaccharide is sized to a weight average molecular weight between 450 kDa and 800 kDa. In an embodiment, the isolated serotype 3 capsular polysaccharide is sized to a weight average molecular weight between 450 kDa and 700 kDa. In an embodiment, the isolated serotype 3 capsular polysaccharide is sized to a weight average molecular weight between 450 kDa and 600 kDa. In an embodiment, the isolated serotype 3 capsular polysaccharide is sized to a weight average molecular weight between 450 kDa and 500 kDa.

In an embodiment, the isolated serotype 3 capsular polysaccharide is sized to a weight average molecular weight between 500 kDa and 1000 kDa. In an embodiment, the isolated serotype 3 capsular polysaccharide is sized to a weight average molecular weight between 500 kDa and 900 kDa. In an embodiment, the isolated serotype 3 capsular polysaccharide is sized to a weight average molecular weight between 500 kDa and 800 kDa. In an embodiment, the isolated serotype 3 capsular polysaccharide is sized to a weight average molecular weight between 500 kDa and 700 kDa. In an embodiment, the isolated serotype 3 capsular polysaccharide is sized to a weight average molecular weight between 500 kDa and 600 kDa.

In an embodiment, the isolated serotype 3 capsular polysaccharide is sized to a weight average molecular weight between 500 kDa and 1000 kDa. In an embodiment, the isolated serotype 3 capsular polysaccharide is sized to a weight average molecular weight between 500 kDa and 900 kDa. In an embodiment, the isolated serotype 3 capsular polysaccharide is sized to a weight average molecular weight between 500 kDa and 800 kDa. In an embodiment, the isolated serotype 3 capsular polysaccharide is sized to a weight average molecular weight between 500 kDa and 700 kDa. In an embodiment, the isolated serotype 3 capsular polysaccharide is sized to a weight average molecular weight between 500 kDa and 600 kDa.

In an embodiment, the isolated serotype 3 capsular polysaccharide is sized to a weight average molecular weight between 600 kDa and 1000 kDa. In an embodiment, the isolated serotype 3 capsular polysaccharide is sized to a weight average molecular weight between 600 kDa and 900 kDa. In an embodiment, the isolated serotype 3 capsular polysaccharide is sized to a weight average molecular weight between 600 kDa and 800 kDa. In an embodiment, the isolated serotype 3 capsular polysaccharide is sized to a weight average molecular weight between 600 kDa and 700 kDa.

In an embodiment, the isolated serotype 3 capsular polysaccharide is sized to a weight average molecular weight between 700 kDa and 1000 kDa. In an embodiment, the isolated serotype 3 capsular polysaccharide is sized to a weight average molecular weight between 700 kDa and 900 kDa. In an embodiment, the isolated serotype 3 capsular polysaccharide is sized to a weight average molecular weight between 700 kDa and 800 kDa.

In an embodiment, the isolated serotype 3 capsular polysaccharide is sized to a weight average molecular weight between 800 kDa and 1000 kDa. In an embodiment, the isolated serotype 3 capsular polysaccharide is sized to a weight average molecular weight between 800 kDa and 900 kDa.

In an embodiment, the isolated serotype 3 capsular polysaccharide is sized to a weight average molecular weight between 900 kDa and 1000 kDa.

In a preferred embodiment, the isolated serotype 3 capsular polysaccharide is sized to a weight average molecular weight between 100 kDa and 200 kDa. In an even preferred embodiment, the isolated serotype 3 capsular polysaccharide is sized to a weight average molecular weight between 120 kDa and 180 kDa. Even more preferably, the isolated serotype 3 capsular polysaccharide is sized to a weight average molecular weight between 140 kDa and 160 kDa. In a preferred embodiment, the isolated serotype 3 capsular polysaccharide is sized to a weight average molecular weight of about 150 kDa. In an embodiment, the isolated serotype 3 capsular polysaccharide is sized to a weight average molecular weight of about 5 kDa. In an embodiment, the isolated serotype 3 capsular polysaccharide is sized to a weight average molecular weight of about 50 kDa. In an embodiment, the isolated serotype 3 capsular polysaccharide is sized to a weight average molecular weight of about 100 kDa. In an embodiment, the isolated serotype 3 capsular polysaccharide is sized to a weight average molecular weight of about 110 kDa. In an embodiment, the isolated serotype 3 capsular polysaccharide is sized to a weight average molecular weight of about 120 kDa. In an embodiment, the isolated serotype 3 capsular polysaccharide is sized to a weight average molecular weight of about 130 kDa. In an embodiment, the isolated serotype 3 capsular polysaccharide is sized to a weight average molecular weight of about 140 kDa. In an embodiment, the isolated serotype 3 capsular polysaccharide is sized to a weight average molecular weight of about 150 kDa. In an embodiment, the isolated serotype 3 capsular polysaccharide is sized to a weight average molecular weight of about 160 kDa. In an embodiment, the isolated serotype 3 capsular polysaccharide is sized to a weight average molecular weight of about 170 kDa. In an embodiment, the isolated serotype 3 capsular polysaccharide is sized to a weight average molecular weight of about 180 kDa. In an embodiment, the isolated serotype 3 capsular polysaccharide is sized to a weight average molecular weight of about 190 kDa. In an embodiment, the isolated serotype 3 capsular polysaccharide is sized to a weight average molecular weight of about 200 kDa. In an embodiment, the isolated serotype 3 capsular polysaccharide is sized to a weight average molecular weight of about 250 kDa. In an embodiment, the isolated serotype 3 capsular polysaccharide is sized to a weight average molecular weight of about 300 kDa. In an embodiment, the isolated serotype 3 capsular polysaccharide is sized to a weight average molecular weight of about 350 kDa. In an embodiment, the isolated serotype 3 capsular polysaccharide is sized to a weight average molecular weight of about 400 kDa. In an embodiment, the isolated serotype 3 capsular polysaccharide is sized to a weight average molecular weight of about 450 kDa. In an embodiment, the isolated serotype 3 capsular polysaccharide is sized to a weight average molecular weight of about 500 kDa. In an embodiment, the isolated serotype 3 capsular polysaccharide is sized to a weight average molecular weight of about 550 kDa. In an embodiment, the isolated serotype 3 capsular polysaccharide is sized to a weight average molecular weight of about 600 kDa. In an embodiment, the isolated serotype 3 capsular polysaccharide is sized to a weight average molecular weight of about 700 kDa. In an embodiment, the isolated serotype 3 capsular polysaccharide is sized to a weight average molecular weight of about 800 kDa. In an embodiment, the isolated serotype 3 capsular polysaccharide is sized to a weight average molecular weight of about 900 kDa. In an embodiment, the isolated serotype 3 capsular polysaccharide is sized to a weight average molecular weight of about 1000 kDa.

In an embodiment, the isolated serotype 3 capsular polysaccharide is not sized.

The isolated serotype 3 capsular polysaccharide described above may be activated (e.g., chemically activated) to make them capable of reacting (e.g. with a linker or directly with the carrier protein) and then incorporated into glycoconjugates, as further described herein.

For the purposes of the invention the term ‘glycoconjugate’ indicates a saccharide covalently linked to a carrier protein. In one embodiment a saccharide is linked directly to a carrier protein. In a second embodiment a saccharide is linked to a carrier protein through a spacer/linker.

In general, covalent conjugation of saccharides to carriers enhances the immunogenicity of saccharides as it converts them from T-independent antigens to T-dependent antigens, thus allowing priming for immunological memory. Conjugation is particularly useful for pediatric vaccines.

1.2 Streptococcus pneumoniae Serotype 3 Glycoconjugates of the Invention

In some embodiments, the serotype 3 glycoconjugate of the present invention comprises a serotype 3 capsular polysaccharide wherein the weight average molecular weight (Mw) of said polysaccharide before conjugation is between 10 kDa and 2,000 kDa. The weight average molecular weight (Mw) of the saccharide before conjugation refers to the Mw before the activation of the polysaccharide (i.e. after an eventual sizing step but before reacting the polysaccharide with an activating agent). In the context of the present invention the Mw of the polysaccharide is not substantially modified by the activation step and the Mw of the polysaccharide incorporated in the conjugate is similar to the Mw of the polysaccharide as measured before activation.

In an embodiment, the polysaccharide is activated with an oxidizing agent which oxidizes a terminal hydroxyl group to an aldehyde (see section 1.3 below). In another embodiment, the polysaccharide is activated with CDI or CDT (see sections 1.4 below).

In an embodiment, the serotype 3 glycoconjugate of the present invention comprises a serotype 3 capsular polysaccharide wherein the weight average molecular weight (Mw) of said polysaccharide before conjugation is between 50 kDa and 1,000 kDa. In an embodiment, the weight average molecular weight (Mw) is between 50 kDa and 750 kDa. In an embodiment, the weight average molecular weight (Mw) is between 50 kDa and 700 kDa. In an embodiment, the weight average molecular weight (Mw) is between 50 kDa and 600 kDa. In an embodiment, the weight average molecular weight (Mw) is between 50 kDa and 500 kDa. In an embodiment, the weight average molecular weight (Mw) is between 50 kDa and 400 kDa. In an embodiment, the weight average molecular weight (Mw) is between 50 kDa and 300 kDa. In an embodiment, the weight average molecular weight (Mw) is between 50 kDa and 200 kDa. In an embodiment, the weight average molecular weight (Mw) is between 50 kDa and 150 kDa. In an embodiment, the weight average molecular weight (Mw) is between 50 kDa and 140 kDa. In an embodiment, the weight average molecular weight (Mw) is between 50 kDa and 130 kDa. In an embodiment, the weight average molecular weight (Mw) is between 50 kDa and 120 kDa. In an embodiment, the weight average molecular weight (Mw) is between 50 kDa and 110 kDa.

In an embodiment, the serotype 3 glycoconjugate of the present invention comprises a serotype 3 capsular polysaccharide wherein the weight average molecular weight (Mw) of said polysaccharide before conjugation is between 75 kDa and 1,000 kDa. In an embodiment, the weight average molecular weight (Mw) is between 75 kDa and 750 kDa.

In an embodiment, the weight average molecular weight (Mw) is between 75 kDa and 700 kDa. In an embodiment, the weight average molecular weight (Mw) is between 75 kDa and 600 kDa. In an embodiment, the weight average molecular weight (Mw) is between 75 kDa and 500 kDa. In an embodiment, the weight average molecular weight (Mw) is between 75 kDa and 400 kDa. In an embodiment, the weight average molecular weight (Mw) is between 75 kDa and 300 kDa. In an embodiment, the weight average molecular weight (Mw) is between 75 kDa and 200 kDa. In an embodiment, the weight average molecular weight (Mw) is between 75 kDa and 150 kDa. In an embodiment, the weight average molecular weight (Mw) is between 75 kDa and 140 kDa. In an embodiment, the weight average molecular weight (Mw) is between 75 kDa and 130 kDa.

In an embodiment, the weight average molecular weight (Mw) is between 75 kDa and 120 kDa. In an embodiment, the weight average molecular weight (Mw) is between 75 kDa and 110 kDa.

In an embodiment, the serotype 3 glycoconjugate of the present invention comprises a serotype 3 capsular polysaccharide wherein the weight average molecular weight (Mw) of said polysaccharide before conjugation is between 100 kDa and 1,000 kDa. In an embodiment, the weight average molecular weight (Mw) is between 100 kDa and 750 kDa. In an embodiment, the weight average molecular weight (Mw) is between 100 kDa and 700 kDa. In an embodiment, the weight average molecular weight (Mw) is between 100 kDa and 600 kDa. In an embodiment, the weight average molecular weight (Mw) is between 100 kDa and 500 kDa. In an embodiment, the weight average molecular weight (Mw) is between 100 kDa and 400 kDa. In an embodiment, the weight average molecular weight (Mw) is between 100 kDa and 300 kDa. In an embodiment, the weight average molecular weight (Mw) is between 100 kDa and 200 kDa. In an embodiment, the weight average molecular weight (Mw) is between 100 kDa and 175 kDa. In a preferred embodiment, the weight average molecular weight (Mw) is between 100 kDa and 160 kDa. In an embodiment, the weight average molecular weight (Mw) is between 100 kDa and 150 kDa. In an embodiment, the weight average molecular weight (Mw) is between 100 kDa and 140 kDa. In an embodiment, the weight average molecular weight (Mw) is between 100 kDa and 130 kDa. In an embodiment, the weight average molecular weight (Mw) is between 100 kDa and 120 kDa. In an embodiment, the weight average molecular weight (Mw) is between 100 kDa and 110 kDa.

In an embodiment, the serotype 3 glycoconjugate of the present invention comprises a serotype 3 capsular polysaccharide wherein the weight average molecular weight (Mw) of said polysaccharide before conjugation is between 125 kDa and 1,000 kDa. In an embodiment, the weight average molecular weight (Mw) is between 125 kDa and 750 kDa. In an embodiment, the weight average molecular weight (Mw) is between 125 kDa and 700 kDa. In an embodiment, the weight average molecular weight (Mw) is between 125 kDa and 600 kDa. In an embodiment, the weight average molecular weight (Mw) is between 125 kDa and 500 kDa. In an embodiment, the weight average molecular weight (Mw) is between 125 kDa and 400 kDa. In an embodiment, the weight average molecular weight (Mw) is between 125 kDa and 300 kDa. In an embodiment, the weight average molecular weight (Mw) is between 125 kDa and 200 kDa. In an embodiment, the weight average molecular weight (Mw) is between 125 kDa and 150 kDa. In an embodiment, the weight average molecular weight (Mw) is between 125 kDa and 140 kDa. In an embodiment, the weight average molecular weight (Mw) is between 125 kDa and 130 kDa.

In an embodiment, the serotype 3 glycoconjugate of the present invention comprises a serotype 3 capsular polysaccharide wherein the weight average molecular weight (Mw) of said polysaccharide before conjugation is between 130 kDa and 1,000 kDa. In an embodiment, the weight average molecular weight (Mw) is between 130 kDa and 750 kDa. In an embodiment, the weight average molecular weight (Mw) is between 130 kDa and 700 kDa. In an embodiment, the weight average molecular weight (Mw) is between 130 kDa and 600 kDa. In an embodiment, the weight average molecular weight (Mw) is between 130 kDa and 500 kDa. In an embodiment, the weight average molecular weight (Mw) is between 130 kDa and 400 kDa. In an embodiment, the weight average molecular weight (Mw) is between 130 kDa and 300 kDa. In an embodiment, the weight average molecular weight (Mw) is between 130 kDa and 200 kDa. In an embodiment, the weight average molecular weight (Mw) is between 130 kDa and 160 kDa. In an embodiment, the weight average molecular weight (Mw) is between 130 kDa and 150 kDa. In an embodiment, the weight average molecular weight (Mw) is between 130 kDa and 140 kDa.

In an embodiment, the serotype 3 glycoconjugate of the present invention comprises a serotype 3 capsular polysaccharide wherein the weight average molecular weight (Mw) of said polysaccharide before conjugation is between 150 kDa and 1,000 kDa. In an embodiment, the weight average molecular weight (Mw) is between 150 kDa and 750 kDa. In an embodiment, the weight average molecular weight (Mw) is between 150 kDa and 700 kDa. In an embodiment, the weight average molecular weight (Mw) is between 150 kDa and 600 kDa. In an embodiment, the weight average molecular weight (Mw) is between 150 kDa and 500 kDa. In an embodiment, the weight average molecular weight (Mw) is between 150 kDa and 400 kDa. In an embodiment, the weight average molecular weight (Mw) is between 150 kDa and 300 kDa. In an embodiment, the weight average molecular weight (Mw) is between 150 kDa and 200 kDa.

In an embodiment, the serotype 3 glycoconjugate of the present invention comprises a serotype 3 capsular polysaccharide wherein the weight average molecular weight (Mw) of said polysaccharide before conjugation is between 200 kDa and 1,000 kDa. In an embodiment, the weight average molecular weight (Mw) is between 200 kDa and 750 kDa. In an embodiment, the weight average molecular weight (Mw) is between 200 kDa and 700 kDa. In an embodiment, the weight average molecular weight (Mw) is between 200 kDa and 600 kDa. In an embodiment, the weight average molecular weight (Mw) is between 200 kDa and 500 kDa. In an embodiment, the weight average molecular weight (Mw) is between 200 kDa and 400 kDa. In an embodiment, the weight average molecular weight (Mw) is between 200 kDa and 300 kDa.

In an embodiment, the serotype 3 glycoconjugate of the present invention comprises a serotype 3 capsular polysaccharide wherein the weight average molecular weight (Mw) of said polysaccharide before conjugation is between 300 kDa and 1,000 kDa. In an embodiment, the weight average molecular weight (Mw) is between 300 kDa and 750 kDa. In an embodiment, the weight average molecular weight (Mw) is between 300 kDa and 700 kDa. In an embodiment, the weight average molecular weight (Mw) is between 300 kDa and 600 kDa. In an embodiment, the weight average molecular weight (Mw) is between 300 kDa and 500 kDa. In an embodiment, the weight average molecular weight (Mw) is between 300 kDa and 400 kDa.

In an embodiment, the serotype 3 glycoconjugate of the present invention comprises a serotype 3 capsular polysaccharide wherein the weight average molecular weight (Mw) of said polysaccharide before conjugation is between 400 kDa and 1,000 kDa. In an embodiment, the weight average molecular weight (Mw) is between 400 kDa and 750 kDa. In an embodiment, the weight average molecular weight (Mw) is between 400 kDa and 700 kDa. In an embodiment, the weight average molecular weight (Mw) is between 400 kDa and 600 kDa. In an embodiment, the weight average molecular weight (Mw) is between 400 kDa and 500 kDa.

In an embodiment, the serotype 3 glycoconjugate of the present invention comprises a serotype 3 capsular polysaccharide wherein the weight average molecular weight (Mw) of said polysaccharide before conjugation is between 500 kDa and 1,000 kDa. In an embodiment, the weight average molecular weight (Mw) is between 500 kDa and 750 kDa. In an embodiment, the weight average molecular weight (Mw) is between 500 kDa and 700 kDa. In an embodiment, the weight average molecular weight (Mw) is between 500 kDa and 600 kDa.

In an embodiment, the serotype 3 glycoconjugate of the present invention comprises a serotype 3 capsular polysaccharide wherein the weight average molecular weight (Mw) of said polysaccharide before conjugation is between 600 kDa and 1,000 kDa. In an embodiment, the weight average molecular weight (Mw) is between 600 kDa and 750 kDa. In an embodiment, the weight average molecular weight (Mw) is between 600 kDa and 700 kDa.

In an embodiment, the serotype 3 glycoconjugate of the present invention comprises a serotype 3 capsular polysaccharide wherein the weight average molecular weight (Mw) of said polysaccharide before conjugation is between 700 kDa and 1,000 kDa. In an embodiment, the weight average molecular weight (Mw) is between 700 kDa and 750 kDa.

In an embodiment, the serotype 3 glycoconjugate of the present invention comprises a serotype 3 capsular polysaccharide wherein the weight average molecular weight (Mw) of said polysaccharide before conjugation is between 750 kDa and 1,000 kDa. In a preferred embodiment, the serotype 3 glycoconjugate of the present invention comprises a serotype 3 capsular polysaccharide wherein the weight average molecular weight (Mw) of said polysaccharide before conjugation is between 100 kDa and 200 kDa. In an even preferred embodiment, the weight average molecular weight (Mw) is between 120 kDa and 180 kDa. In an even preferred embodiment, the weight average molecular weight (Mw) is between 130 kDa and 170 kDa. In an even preferred embodiment, the weight average molecular weight (Mw) is between 140 kDa and 160 kDa. Even more preferably, the weight average molecular weight (Mw) is about 150 kDa.

Any whole number integer within any of the above ranges is contemplated as an embodiment of the disclosure.

In an embodiment, the serotype 3 glycoconjugate of the present invention comprises a serotype 3 capsular polysaccharide wherein the weight average molecular weight (Mw) of said polysaccharide before conjugation is about 1,000 kDa. In an embodiment, the weight average molecular weight (Mw) is about 750 kDa. In an embodiment, the weight average molecular weight (Mw) is about 700 kDa. In an embodiment, the weight average molecular weight (Mw) is about 600 kDa. In an embodiment, the weight average molecular weight (Mw) is about 500 kDa. In an embodiment, the weight average molecular weight (Mw) is about 400 kDa. In an embodiment, the weight average molecular weight (Mw) is about 300 kDa. In an embodiment, the weight average molecular weight (Mw) is about 200 kDa. In an embodiment, the weight average molecular weight (Mw) is about 150 kDa. In an embodiment, the weight average molecular weight (Mw) is about 140 kDa. In a preferred embodiment, the weight average molecular weight (Mw) is about 130 kDa. In an embodiment, the weight average molecular weight (Mw) is about 120 kDa. In an embodiment, the weight average molecular weight (Mw) is about 110 kDa. In an embodiment, the weight average molecular weight (Mw) is about 100 kDa.

In some such embodiments, the serotype 3 glycoconjugates are prepared using reductive amination chemistry (see section 1.3). In other embodiments, the serotype 3 glycoconjugates are prepared using CDI and/or CDT chemistry (see section 1.4).

In some embodiments, the serotype 3 glycoconjugate of the invention has a weight average molecular weight (Mw) of between 250 kDa and 15,000 kDa. In other embodiments, the serotype 3 glycoconjugate has a weight average molecular weight (Mw) of between 500 kDa and 10,000 kDa.

In other embodiments, the serotype 3 glycoconjugate has a weight average molecular weight (Mw) of between 250 kDa and 10,000 kDa. In other embodiments, the serotype 3 glycoconjugate has a weight average molecular weight (Mw) of between 250 kDa and 9,000 kDa. In other embodiments, the serotype 3 glycoconjugate has a weight average molecular weight (Mw) of between 250 kDa and 8,000 kDa. In still other embodiments, the serotype 3 glycoconjugate has a weight average molecular weight (Mw) of between 250 kDa and 7,000 kDa. In still other embodiments, the serotype 3 glycoconjugate has a weight average molecular weight (Mw) of between 250 kDa and 6,000 kDa. In still other embodiments, the serotype 3 glycoconjugate has a weight average molecular weight (Mw) of between 250 kDa and 5,000 kDa. In still other embodiments, the serotype 3 glycoconjugate has a weight average molecular weight (Mw) of between 250 kDa and 4,000 kDa. In still other embodiments, the serotype 3 glycoconjugate has a weight average molecular weight (Mw) of between 250 kDa and 3,000 kDa. In still other embodiments, the serotype 3 glycoconjugate has a weight average molecular weight (Mw) of between 250 kDa and 2,000 kDa. In still other embodiments, the serotype 3 glycoconjugate has a weight average molecular weight (Mw) of between 250 kDa and 1,500 kDa. In still other embodiments, the serotype 3 glycoconjugate has a weight average molecular weight (Mw) of between 250 kDa and 1,000 kDa. In still other embodiments, the serotype 3 glycoconjugate has a weight average molecular weight (Mw) of between 250 kDa and 750 kDa. In still other embodiments, the serotype 3 glycoconjugate has a weight average molecular weight (Mw) of between 250 kDa and 600 kDa. In still other embodiments, the serotype 3 glycoconjugate has a weight average molecular weight (Mw) of between 250 kDa and 500 kDa. In still other embodiments, the serotype 3 glycoconjugate has a weight average molecular weight (Mw) of between 250 kDa and 400 kDa.

In other embodiments, the serotype 3 glycoconjugate has a weight average molecular weight (Mw) of between 500 kDa and 10,000 kDa. In other embodiments, the serotype 3 glycoconjugate has a weight average molecular weight (Mw) of between 500 kDa and 9,000 kDa. In other embodiments, the serotype 3 glycoconjugate has a weight average molecular weight (Mw) of between 500 kDa and 8,000 kDa. In still other embodiments, the serotype 3 glycoconjugate has a weight average molecular weight (Mw) of between 500 kDa and 7,000 kDa. In still other embodiments, the serotype 3 glycoconjugate has a weight average molecular weight (Mw) of between 500 kDa and 6,000 kDa. In still other embodiments, the serotype 3 glycoconjugate has a weight average molecular weight (Mw) of between 500 kDa and 5,000 kDa. In still other embodiments, the serotype 3 glycoconjugate has a weight average molecular weight (Mw) of between 500 kDa and 4,000 kDa. In still other embodiments, the serotype 3 glycoconjugate has a weight average molecular weight (Mw) of between 500 kDa and 3,000 kDa. In still other embodiments, the serotype 3 glycoconjugate has a weight average molecular weight (Mw) of between 500 kDa and 2,000 kDa. In still other embodiments, the serotype 3 glycoconjugate has a weight average molecular weight (Mw) of between 500 kDa and 1,500 kDa. In still other embodiments, the serotype 3 glycoconjugate has a weight average molecular weight (Mw) of between 500 kDa and 1,000 kDa. In still other embodiments, the serotype 3 glycoconjugate has a weight average molecular weight (Mw) of between 500 kDa and 750 kDa. In still other embodiments, the serotype 3 glycoconjugate has a weight average molecular weight (Mw) of between 500 kDa and 600 kDa.

In other embodiments, the serotype 3 glycoconjugate has a weight average molecular weight (Mw) of between 750 kDa and 10,000 kDa. In other embodiments, the serotype 3 glycoconjugate has a weight average molecular weight (Mw) of between 750 kDa and 9,000 kDa. In other embodiments, the serotype 3 glycoconjugate has a weight average molecular weight (Mw) of between 750 kDa and 8,000 kDa. In still other embodiments, the serotype 3 glycoconjugate has a weight average molecular weight (Mw) of between 750 kDa and 7,000 kDa. In still other embodiments, the serotype 3 glycoconjugate has a weight average molecular weight (Mw) of between 750 kDa and 6,000 kDa. In still other embodiments, the serotype 3 glycoconjugate has a weight average molecular weight (Mw) of between 750 kDa and 5,000 kDa. In still other embodiments, the serotype 3 glycoconjugate has a weight average molecular weight (Mw) of between 750 kDa and 4,000 kDa. In still other embodiments, the serotype 3 glycoconjugate has a weight average molecular weight (Mw) of between 750 kDa and 3,000 kDa. In still other embodiments, the serotype 3 glycoconjugate has a weight average molecular weight (Mw) of between 750 kDa and 2,000 kDa. In still other embodiments, the serotype 3 glycoconjugate has a weight average molecular weight (Mw) of between 750 kDa and 1,500 kDa. In still other embodiments, the serotype 3 glycoconjugate has a weight average molecular weight (Mw) of between 750 kDa and 1,000 kDa.

In other embodiments, the serotype 3 glycoconjugate has a weight average molecular weight (Mw) of between 1,000 kDa and 10,000 kDa. In other embodiments, the serotype 3 glycoconjugate has a weight average molecular weight (Mw) of between 1,000 kDa and 9,000 kDa. In other embodiments, the serotype 3 glycoconjugate has a weight average molecular weight (Mw) of between 1,000 kDa and 8,000 kDa. In still other embodiments, the serotype 3 glycoconjugate has a weight average molecular weight (Mw) of between 1,000 kDa and 7,000 kDa. In still other embodiments, the serotype 3 glycoconjugate has a weight average molecular weight (Mw) of between 1,000 kDa and 6,000 kDa. In still other embodiments, the serotype 3 glycoconjugate has a weight average molecular weight (Mw) of between 1,000 kDa and 5,000 kDa. In still other embodiments, the serotype 3 glycoconjugate has a weight average molecular weight (Mw) of between 1,000 kDa and 4,000 kDa. In still other embodiments, the serotype 3 glycoconjugate has a weight average molecular weight (Mw) of between 1,000 kDa and 3,000 kDa. In still other embodiments, the serotype 3 glycoconjugate has a weight average molecular weight (Mw) of between 1,000 kDa and 2,000 kDa. In still other embodiments, the serotype 3 glycoconjugate has a weight average molecular weight (Mw) of between 1,000 kDa and 1,500 kDa.

In other embodiments, the serotype 3 glycoconjugate has a weight average molecular weight (Mw) of between 2,000 kDa and 10,000 kDa. In other embodiments, the serotype 3 glycoconjugate has a weight average molecular weight (Mw) of between 2,000 kDa and 9,000 kDa. In other embodiments, the serotype 3 glycoconjugate has a weight average molecular weight (Mw) of between 2,000 kDa and 8,000 kDa. In still other embodiments, the serotype 3 glycoconjugate has a weight average molecular weight (Mw) of between 2,000 kDa and 7,000 kDa. In still other embodiments, the serotype 3 glycoconjugate has a weight average molecular weight (Mw) of between 2,000 kDa and 6,000 kDa. In still other embodiments, the serotype 3 glycoconjugate has a weight average molecular weight (Mw) of between 2,000 kDa and 5,000 kDa. In still other embodiments, the serotype 3 glycoconjugate has a weight average molecular weight (Mw) of between 2,000 kDa and 4,000 kDa. In still other embodiments, the serotype 3 glycoconjugate has a weight average molecular weight (Mw) of between 2,000 kDa and 3,000 kDa. In still other embodiments, the serotype 3 glycoconjugate has a weight average molecular weight (Mw) of between 2,000 kDa and 3,500 kDa.

In still other embodiments, the serotype 3 glycoconjugate has a weight average molecular weight (Mw) of between 2,250 kDa and 3,500 kDa.

In other embodiments, the serotype 3 glycoconjugate has a weight average molecular weight (Mw) of between 3,000 kDa and 10,000 kDa. In other embodiments, the serotype 3 glycoconjugate has a weight average molecular weight (Mw) of between 3,000 kDa and 9,000 kDa. In other embodiments, the serotype 3 glycoconjugate has a weight average molecular weight (Mw) of between 3,000 kDa and 8,000 kDa. In still other embodiments, the serotype 3 glycoconjugate has a weight average molecular weight (Mw) of between 3,000 kDa and 7,000 kDa. In still other embodiments, the serotype 3 glycoconjugate has a weight average molecular weight (Mw) of between 3,000 kDa and 6,000 kDa. In still other embodiments, the serotype 3 glycoconjugate has a weight average molecular weight (Mw) of between 3,000 kDa and 5,000 kDa. In still other embodiments, the serotype 3 glycoconjugate has a weight average molecular weight (Mw) of between 3,000 kDa and 4,000 kDa.

In other embodiments, the serotype 3 glycoconjugate has a weight average molecular weight (Mw) of between 4,000 kDa and 10,000 kDa. In other embodiments, the serotype 3 glycoconjugate has a weight average molecular weight (Mw) of between 4,000 kDa and 9,000 kDa. In other embodiments, the serotype 3 glycoconjugate has a weight average molecular weight (Mw) of between 4,000 kDa and 8,000 kDa. In still other embodiments, the serotype 3 glycoconjugate has a weight average molecular weight (Mw) of between 4,000 kDa and 7,000 kDa. In still other embodiments, the serotype 3 glycoconjugate has a weight average molecular weight (Mw) of between 4,000 kDa and 6,000 kDa. In still other embodiments, the serotype 3 glycoconjugate has a weight average molecular weight (Mw) of between 4,000 kDa and 5,000 kDa.

In other embodiments, the serotype 3 glycoconjugate has a weight average molecular weight (Mw) of between 5,000 kDa and 10,000 kDa. In other embodiments, the serotype 3 glycoconjugate has a weight average molecular weight (Mw) of between 5,000 kDa and 9,000 kDa. In other embodiments, the serotype 3 glycoconjugate has a weight average molecular weight (Mw) of between 5,000 kDa and 8,000 kDa. In still other embodiments, the serotype 3 glycoconjugate has a weight average molecular weight (Mw) of between 5,000 kDa and 7,000 kDa. In still other embodiments, the serotype 3 glycoconjugate has a weight average molecular weight (Mw) of between 5,000 kDa and 6,000 kDa.

In other embodiments, the serotype 3 glycoconjugate has a weight average molecular weight (Mw) of between 6,000 kDa and 10,000 kDa. In other embodiments, the serotype 3 glycoconjugate has a weight average molecular weight (Mw) of between 6,000 kDa and 9,000 kDa. In other embodiments, the serotype 3 glycoconjugate has a weight average molecular weight (Mw) of between 6,000 kDa and 8,000 kDa. In still other embodiments, the serotype 3 glycoconjugate has a weight average molecular weight (Mw) of between 6,000 kDa and 7,000 kDa.

In other embodiments, the serotype 3 glycoconjugate has a weight average molecular weight (Mw) of between 7,000 kDa and 10,000 kDa. In other embodiments, the serotype 3 glycoconjugate has a weight average molecular weight (Mw) of between 7,000 kDa and 9,000 kDa. In other embodiments, the serotype 3 glycoconjugate has a weight average molecular weight (Mw) of between 7,000 kDa and 8,000 kDa.

In other embodiments, the serotype 3 glycoconjugate has a weight average molecular weight (Mw) of between 8,000 kDa and 10,000 kDa. In other embodiments, the serotype 3 glycoconjugate has a weight average molecular weight (Mw) of between 8,000 kDa and 9,000 kDa.

In other embodiments, the serotype 3 glycoconjugate has a weight average molecular weight (Mw) of between 9,000 kDa and 10,000 kDa.

Any whole number integer within any of the above ranges is contemplated as an embodiment of the disclosure.

In other embodiments, the serotype 3 glycoconjugate has a weight average molecular weight (Mw) of about 10,000 kDa. In other embodiments, the serotype 3 glycoconjugate has a weight average molecular weight (Mw) of about 9,000 kDa. In other embodiments, the serotype 3 glycoconjugate has a weight average molecular weight (Mw) of about 8,000 kDa. In still other embodiments, the serotype 3 glycoconjugate has a weight average molecular weight (Mw) of about 7,000 kDa. In still other embodiments, the serotype 3 glycoconjugate has a weight average molecular weight (Mw) of about 6,000 kDa. In still other embodiments, the serotype 3 glycoconjugate has a weight average molecular weight (Mw) of about 5,000 kDa. In still other embodiments, the serotype 3 glycoconjugate has a weight average molecular weight (Mw) of about 4,000 kDa. In still other embodiments, the serotype 3 glycoconjugate has a weight average molecular weight (Mw) of about 3,500 kDa. In still other embodiments, the serotype 3 glycoconjugate has a weight average molecular weight (Mw) of about 3,250 kDa. In still other embodiments, the serotype 3 glycoconjugate has a weight average molecular weight (Mw) of about 3,000 kDa. In still other embodiments, the serotype 3 glycoconjugate has a weight average molecular weight (Mw) of about 2,500 kDa. In still other embodiments, the serotype 3 glycoconjugate has a weight average molecular weight (Mw) of about 2,250 kDa. In still other embodiments, the serotype 3 glycoconjugate has a weight average molecular weight (Mw) of about 2,000 kDa. In still other embodiments, the serotype 3 glycoconjugate has a weight average molecular weight (Mw) of about 1,000 kDa. In still other embodiments, the serotype 3 glycoconjugate has a weight average molecular weight (Mw) of about 750 kDa. In still other embodiments, the serotype 3 glycoconjugate has a weight average molecular weight (Mw) of about 600 kDa. In still other embodiments, the serotype 3 glycoconjugate has a weight average molecular weight (Mw) of about 500 kDa. In still other embodiments, the serotype 3 glycoconjugate has a weight average molecular weight (Mw) of about 400 kDa.

The molecular weight of the polysaccharide can be measured by Size Exclusion Chromatography (SEC) combined with Multiangle Laser Light Scattering detector (MALLS).

Another way to characterize the serotype 3 glycoconjugates of the invention is by the number of lysine residues in the carrier protein (e.g., CRM₁₉₇, TT or SCP) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 3 glycoconjugate of the invention is between 2 and 15. In an embodiment, the degree of conjugation of the serotype 3 glycoconjugate of the invention is between 2 and 13. In an embodiment, the degree of conjugation of the serotype 3 glycoconjugate of the invention is between 2 and 10. In an embodiment, the degree of conjugation of the serotype 3 glycoconjugate of the invention is between 2 and 8. In an embodiment, the degree of conjugation of the serotype 3 glycoconjugate of the invention is between 2 and 6. In an embodiment, the degree of conjugation of the serotype 3 glycoconjugate of the invention is between 2 and 5. In an embodiment, the degree of conjugation of the serotype 3 glycoconjugate of the invention is between 2 and 4. In an embodiment, the degree of conjugation of the serotype 3 glycoconjugate of the invention is between 3 and 15. In an embodiment, the degree of conjugation of the serotype 3 glycoconjugate of the invention is between 3 and 13. In an embodiment, the degree of conjugation of the serotype 3 glycoconjugate of the invention is between 3 and 10. In an embodiment, the degree of conjugation of the serotype 3 glycoconjugate of the invention is between 3 and 8. In an embodiment, the degree of conjugation of the serotype 3 glycoconjugate of the invention is between 3 and 6. In an embodiment, the degree of conjugation of the serotype 3 glycoconjugate of the invention is between 3 and 5. In an embodiment, the degree of conjugation of the serotype 3 glycoconjugate of the invention is between 3 and 4. In an embodiment, the degree of conjugation of the serotype 3 glycoconjugate of the invention is between 5 and 15. In an embodiment, the degree of conjugation of the serotype 3 glycoconjugate of the invention is between 5 and 10. In an embodiment, the degree of conjugation of the serotype 3 glycoconjugate of the invention is between 8 and 15. In an embodiment, the degree of conjugation of the serotype 3 glycoconjugate of the invention is between 8 and 12. In an embodiment, the degree of conjugation of the serotype 3 glycoconjugate of the invention is between 10 and 15. In an embodiment, the degree of conjugation of the serotype 3 glycoconjugate of the invention is between 10 and 12.

In an embodiment, the degree of conjugation of the serotype 3 glycoconjugate of the invention is about 2.

In an embodiment, the degree of conjugation of the serotype 3 glycoconjugate of the invention is about 3. In an embodiment, the degree of conjugation of the serotype 3 glycoconjugate of the invention is about 4. In an embodiment, the degree of conjugation of the serotype 3 glycoconjugate of the invention is about 5. In an embodiment, the degree of conjugation of the serotype 3 glycoconjugate of the invention is about 6. In an embodiment, the degree of conjugation of the serotype 3 glycoconjugate of the invention is about 7. In an embodiment, the degree of conjugation of the serotype 3 glycoconjugate of the invention is about 8. In an embodiment, the degree of conjugation of the serotype 3 glycoconjugate of the invention is about 9. In an embodiment, the degree of conjugation of the serotype 3 glycoconjugate of the invention is about 10, about 11. In an embodiment, the degree of conjugation of the serotype 3 glycoconjugate of the invention is about 12. In an embodiment, the degree of conjugation of the serotype 3 glycoconjugate of the invention is about 13. In an embodiment, the degree of conjugation of the serotype 3 glycoconjugate of the invention is about 14. In an embodiment, the degree of conjugation of the serotype 3 glycoconjugate of the invention is about 15. In a preferred embodiment, the degree of conjugation of the serotype 3 glycoconjugate of the invention is between 4 and 7. In some such embodiments, the carrier protein is CRM₁₉₇. In other such embodiments, the carrier protein is SCP.

The serotype 3 glycoconjugates of the invention may also be characterized by the ratio (weight/weight) of saccharide to carrier protein. In some embodiments, the ratio of serotype 3 polysaccharide to carrier protein in the glycoconjugate (w/w) is between 0.5 and 3.0. In other embodiments, the saccharide to carrier protein ratio (w/w) is between 0.5 and 2.0. In other embodiments, the saccharide to carrier protein ratio (w/w) is between 0.5 and 1.5. In other embodiments, the saccharide to carrier protein ratio (w/w) is between 0.8 and 1.2. In other embodiments, the saccharide to carrier protein ratio (w/w) is between 0.5 and 1.0. In other embodiments, the saccharide to carrier protein ratio (w/w) is between 1.0 and 1.5. In other embodiments, the saccharide to carrier protein ratio (w/w) is between 1.0 and 2.0. In further embodiments, the saccharide to carrier protein ratio (w/w) is between 0.8 and 1.2. In a preferred embodiment, the ratio of serotype 3 capsular polysaccharide to carrier protein in the conjugate is between 0.9 and 1.1. In an embodiment, the saccharide to carrier protein ratio (w/w) is about 0.5. In other embodiments, the saccharide to carrier protein ratio (w/w) is about 0.6. In other embodiments, the saccharide to carrier protein ratio (w/w) is about 0.7. In other embodiments, the saccharide to carrier protein ratio (w/w) is about 0.8. In other embodiments, the saccharide to carrier protein ratio (w/w) is about 0.9. In other embodiments, the saccharide to carrier protein ratio (w/w) is about 1.0. In other embodiments, the saccharide to carrier protein ratio (w/w) is about 1.1. In other embodiments, the saccharide to carrier protein ratio (w/w) is about 1.2. In other embodiments, the saccharide to carrier protein ratio (w/w) is about 1.3. In other embodiments, the saccharide to carrier protein ratio (w/w) is about 1.4. In other embodiments, the saccharide to carrier protein ratio (w/w) is about 1.5. In other embodiments, the saccharide to carrier protein ratio (w/w) is about 1.6. In other embodiments, the saccharide to carrier protein ratio (w/w) is about 1.7. In other embodiments, the saccharide to carrier protein ratio (w/w) is about 1.8. In other embodiments, the saccharide to carrier protein ratio (w/w) is about 1.9. In other embodiments, the saccharide to carrier protein ratio (w/w) is about 2.0. In other embodiments, the saccharide to carrier protein ratio (w/w) is about 2.1. In other embodiments, the saccharide to carrier protein ratio (w/w) is about 2.2. In other embodiments, the saccharide to carrier protein ratio (w/w) is about 2.5. In other embodiments, the saccharide to carrier protein ratio (w/w) is about 2.8. In other embodiments, the saccharide to carrier protein ratio (w/w) is about 3.0. In some such embodiments, the carrier protein is CRM₁₉₇. In other such embodiments, the carrier protein is SCP. In other such embodiments, the carrier protein is TT.

The serotype 3 glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In a preferred embodiment, the serotype 3 glycoconjugate comprises less than about 50% of free serotype 3 polysaccharide compared to the total amount of serotype 3 polysaccharide. In a preferred embodiment the serotype 3 glycoconjugate comprises less than about 40% of free serotype 3 polysaccharide compared to the total amount of serotype 3 polysaccharide. In a yet preferred embodiment, the serotype 3 glycoconjugate comprises less than about 25% of free serotype 3 polysaccharide compared to the total amount of serotype 3 polysaccharide. In an even preferred embodiment, the serotype 3 glycoconjugate comprises less than about 20% of free serotype 3 polysaccharide compared to the total amount of serotype 3 polysaccharide. In a yet preferred embodiment, the serotype 3 glycoconjugate comprises less than about 15% of free serotype 3 polysaccharide compared to the total amount of serotype 3 polysaccharide. The serotype 3 glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In a preferred embodiment, at least 30% of the serotype 3 glycoconjugate has a K_{d} below or equal to 0.3 in a CL-4B column. In a preferred embodiment, at least 40% of the glycoconjugate has a K_{d} below or equal to 0.3 in a CL-4B column. In a preferred embodiment, at least 45%, 50%, 55%, 60%, 65%, 70%, 75%, 80%, or 85% of the serotype 3 glycoconjugate has a K_{d} below or equal to 0.3 in a CL-4B column. In a preferred embodiment, at least 60% of the serotype 3 glycoconjugate has a K_{d} below or equal to 0.3 in a CL-4B column. In a preferred embodiment, between 50% and 80% of the serotype 3 glycoconjugate has a K_{d} below or equal to 0.3 in a CL-4B column. In a preferred embodiment, between 65% and 80% of the serotype 3 glycoconjugate has a K_{d} below or equal to 0.3 in a CL-4B column.

1.3 Streptococcus pneumoniae Serotype 3 Glycoconjugates of the Invention Prepared Using Reductive Amination

In an embodiment, serotype 3 glycoconjugates of the present invention are prepared using reductive amination.

The invention also relates to a method of making a serotype 3 glycoconjugate, as disclosed herein.

According to the present invention, reductive amination involves two steps, (1) oxidation (activation) of a purified saccharide, (2) reduction of the activated saccharide and a carrier protein (e.g., CRM₁₆₇, TT or SCP) to form a glycoconjugate (see e.g. WO2006/110381, WO2008/079653, WO2008/143709, WO2008/079732, WO2011/110531, WO2012/119972, WO2015110941, WO2015110940, WO2018/144439, WO2018/156491).

As mentioned above, before oxidation, sizing of the polysaccharide to a target molecular weight (MW) range can be performed.

Therefore, in an embodiment, the isolated polysaccharide is sized before oxidation.

In an embodiment, the isolated polysaccharide is sized to any of the target molecular weight (MW) range defined above.

In an embodiment, the isolated serotype 3 capsular polysaccharide is conjugated to a carrier protein by a process comprising the step of:

(a) reacting said isolated polysaccharide with an oxidizing agent;
(b) compounding the activated polysaccharide of step (a) with a carrier protein; and
(c) reacting the compounded activated polysaccharide and carrier protein with a reducing agent to form a glycoconjugate.

In an embodiment, the isolated serotype 3 capsular polysaccharide is conjugated to a carrier protein by a process comprising the step of:

(a) reacting said isolated polysaccharide with an oxidizing agent;
(a′) quenching the oxidation reaction by addition of a quenching agent;
(b) compounding the activated polysaccharide of step (a) or (a′) with a carrier protein; and
(c) reacting the compounded activated polysaccharide and carrier protein with a reducing agent to form a glycoconjugate.

Following the oxidation step (a) the saccharide is said to be activated and is referred to as “activated polysaccharide”.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate).

In an embodiment, the oxidizing agent is periodate in the presence of bivalent cations (see WO2008/143709).

In an embodiment, the oxidizing agent is periodic acid. In an embodiment, the oxidizing agent is periodic acid in the presence of bivalent cations. In an embodiment, the oxidizing agent is periodic acid in the presence of Mg²⁺. In an embodiment, the oxidizing agent is periodic acid in the presence of Ca²⁺. In an embodiment, the oxidizing agent is orthoperiodate.

In a preferred embodiment, the oxidizing agent is sodium periodate. In an embodiment, the periodate used for the oxidation is metaperiodate. In an embodiment the periodate used for the oxidation is sodium metaperiodate.

When a polysaccharide reacts with periodate, periodate oxidises vicinal hydroxyl groups to form carbonyl or aldehyde groups and causes cleavage of a C—C bond. For this reason, the term “reacting a polysaccharide with periodate” includes oxidation of vicinal hydroxyl groups by periodate.

In one embodiment step a) comprises reacting the polysaccharide with 0.01-2 molar equivalents of periodate. In one embodiment step a) comprises reacting the polysaccharide with 0.05-2 molar equivalents of periodate. In one embodiment step a) comprises reacting the polysaccharide with 0.1-2 molar equivalents of periodate. In one embodiment step a) comprises reacting the polysaccharide with 0.2-2 molar equivalents of periodate. In one embodiment step a) comprises reacting the polysaccharide with 0.3-2 molar equivalents of periodate. In one embodiment step a) comprises reacting the polysaccharide with 0.4-2 molar equivalents of periodate. In one embodiment step a) comprises reacting the polysaccharide with 0.5-2 molar equivalents of periodate. In one embodiment step a) comprises reacting the polysaccharide with 0.6-2 molar equivalents of periodate.

In one embodiment step a) comprises reacting the polysaccharide with 0.01-1.9 molar equivalents of periodate. In one embodiment step a) comprises reacting the polysaccharide with 0.05-1.9 molar equivalents of periodate. In one embodiment step a) comprises reacting the polysaccharide with 0.1-1.9 molar equivalents of periodate. In one embodiment step a) comprises reacting the polysaccharide with 0.2-1.9 molar equivalents of periodate. In one embodiment step a) comprises reacting the polysaccharide with 0.3-1.9 molar equivalents of periodate. In one embodiment step a) comprises reacting the polysaccharide with 0.4-1.9 molar equivalents of periodate. In one embodiment step a) comprises reacting the polysaccharide with 0.5-1.9 molar equivalents of periodate. In one embodiment step a) comprises reacting the polysaccharide with 0.6-1.9 molar equivalents of periodate.

In one embodiment step a) comprises reacting the polysaccharide with 0.01-1.8 molar equivalents of periodate. In one embodiment step a) comprises reacting the polysaccharide with 0.05-1.8 molar equivalents of periodate. In one embodiment step a) comprises reacting the polysaccharide with 0.1-1.8 molar equivalents of periodate. In one embodiment step a) comprises reacting the polysaccharide with 0.2-1.8 molar equivalents of periodate. In one embodiment step a) comprises reacting the polysaccharide with 0.3-1.8 molar equivalents of periodate. In one embodiment step a) comprises reacting the polysaccharide with 0.4-1.8 molar equivalents of periodate. In one embodiment step a) comprises reacting the polysaccharide with 0.5-1.8 molar equivalents of periodate. In one embodiment step a) comprises reacting the polysaccharide with 0.6-1.8 molar equivalents of periodate.

In one embodiment step a) comprises reacting the polysaccharide with 0.01-1.7 molar equivalents of periodate. In one embodiment step a) comprises reacting the polysaccharide with 0.05-1.7 molar equivalents of periodate. In one embodiment step a) comprises reacting the polysaccharide with 0.1-1.7 molar equivalents of periodate. In one embodiment step a) comprises reacting the polysaccharide with 0.2-1.7 molar equivalents of periodate. In one embodiment step a) comprises reacting the polysaccharide with 0.3-1.7 molar equivalents of periodate. In one embodiment step a) comprises reacting the polysaccharide with 0.4-1.7 molar equivalents of periodate. In one embodiment step a) comprises reacting the polysaccharide with 0.5-1.7 molar equivalents of periodate. In one embodiment step a) comprises reacting the polysaccharide with 0.6-1.7 molar equivalents of periodate.

In one embodiment step a) comprises reacting the polysaccharide with 0.01-1.6 molar equivalents of periodate. In one embodiment step a) comprises reacting the polysaccharide with 0.05-1.6 molar equivalents of periodate. In one embodiment step a) comprises reacting the polysaccharide with 0.1-1.6 molar equivalents of periodate. In one embodiment step a) comprises reacting the polysaccharide with 0.2-1.6 molar equivalents of periodate. In one embodiment step a) comprises reacting the polysaccharide with 0.3-1.6 molar equivalents of periodate. In one embodiment step a) comprises reacting the polysaccharide with 0.4-1.6 molar equivalents of periodate. In one embodiment step a) comprises reacting the polysaccharide with 0.5-1.6 molar equivalents of periodate. In one embodiment step a) comprises reacting the polysaccharide with 0.6-1.6 molar equivalents of periodate.

In one embodiment step a) comprises reacting the polysaccharide with 0.01-1.5 molar equivalents of periodate. In one embodiment step a) comprises reacting the polysaccharide with 0.05-1.5 molar equivalents of periodate. In one embodiment step a) comprises reacting the polysaccharide with 0.1-1.5 molar equivalents of periodate. In one embodiment step a) comprises reacting the polysaccharide with 0.2-1.5 molar equivalents of periodate. In one embodiment step a) comprises reacting the polysaccharide with 0.3-1.5 molar equivalents of periodate. In one embodiment step a) comprises reacting the polysaccharide with 0.4-1.5 molar equivalents of periodate. In one embodiment step a) comprises reacting the polysaccharide with 0.5-1.5 molar equivalents of periodate. In one embodiment step a) comprises reacting the polysaccharide with 0.6-1.5 molar equivalents of periodate.

In one embodiment step a) comprises reacting the polysaccharide with 0.01-1.4 molar equivalents of periodate. In one embodiment step a) comprises reacting the polysaccharide with 0.05-1.4 molar equivalents of periodate. In one embodiment step a) comprises reacting the polysaccharide with 0.1-1.4 molar equivalents of periodate. In one embodiment step a) comprises reacting the polysaccharide with 0.2-1.4 molar equivalents of periodate. In one embodiment step a) comprises reacting the polysaccharide with 0.3-1.4 molar equivalents of periodate. In one embodiment step a) comprises reacting the polysaccharide with 0.4-1.4 molar equivalents of periodate. In one embodiment step a) comprises reacting the polysaccharide with 0.5-1.4 molar equivalents of periodate. In one embodiment step a) comprises reacting the polysaccharide with 0.6-1.4 molar equivalents of periodate.

In one embodiment step a) comprises reacting the polysaccharide with 0.01-1.3 molar equivalents of periodate. In one embodiment step a) comprises reacting the polysaccharide with 0.05-1.3 molar equivalents of periodate. In one embodiment step a) comprises reacting the polysaccharide with 0.1-1.3 molar equivalents of periodate. In one embodiment step a) comprises reacting the polysaccharide with 0.2-1.3 molar equivalents of periodate. In one embodiment step a) comprises reacting the polysaccharide with 0.3-1.3 molar equivalents of periodate. In one embodiment step a) comprises reacting the polysaccharide with 0.4-1.3 molar equivalents of periodate. In one embodiment step a) comprises reacting the polysaccharide with 0.5-1.3 molar equivalents of periodate. In one embodiment step a) comprises reacting the polysaccharide with 0.6-1.3 molar equivalents of periodate.

In one embodiment step a) comprises reacting the polysaccharide with 0.01-1.2 molar equivalents of periodate. In one embodiment step a) comprises reacting the polysaccharide with 0.05-1.2 molar equivalents of periodate. In one embodiment step a) comprises reacting the polysaccharide with 0.1-1.2 molar equivalents of periodate. In one embodiment step a) comprises reacting the polysaccharide with 0.2-1.2 molar equivalents of periodate. In one embodiment step a) comprises reacting the polysaccharide with 0.3-1.2 molar equivalents of periodate. In one embodiment step a) comprises reacting the polysaccharide with 0.4-1.2 molar equivalents of periodate. In one embodiment step a) comprises reacting the polysaccharide with 0.5-1.2 molar equivalents of periodate. In one embodiment step a) comprises reacting the polysaccharide with 0.6-1.2 molar equivalents of periodate.

In one embodiment step a) comprises reacting the polysaccharide with 0.01-1.1 molar equivalents of periodate. In one embodiment step a) comprises reacting the polysaccharide with 0.05-1.1 molar equivalents of periodate. In one embodiment step a) comprises reacting the polysaccharide with 0.1-1.1 molar equivalents of periodate. In one embodiment step a) comprises reacting the polysaccharide with 0.2-1.1 molar equivalents of periodate. In one embodiment step a) comprises reacting the polysaccharide with 0.3-1.1 molar equivalents of periodate. In one embodiment step a) comprises reacting the polysaccharide with 0.4-1.1 molar equivalents of periodate. In one embodiment step a) comprises reacting the polysaccharide with 0.5-1.1 molar equivalents of periodate. In one embodiment step a) comprises reacting the polysaccharide with 0.6-1.1 molar equivalents of periodate.

In one embodiment step a) comprises reacting the polysaccharide with 0.01-1.0 molar equivalents of periodate. In one embodiment step a) comprises reacting the polysaccharide with 0.05-1.0 molar equivalents of periodate. In one embodiment step a) comprises reacting the polysaccharide with 0.1-1.0 molar equivalents of periodate. In one embodiment step a) comprises reacting the polysaccharide with 0.2-1.0 molar equivalents of periodate. In one embodiment step a) comprises reacting the polysaccharide with 0.3-1.0 molar equivalents of periodate. In one embodiment step a) comprises reacting the polysaccharide with 0.4-1.0 molar equivalents of periodate. In one embodiment step a) comprises reacting the polysaccharide with 0.5-1.0 molar equivalents of periodate. In one embodiment step a) comprises reacting the polysaccharide with 0.6-1.0 molar equivalents of periodate.

In one embodiment step a) comprises reacting the polysaccharide with 0.01-0.9 molar equivalents of periodate. In one embodiment step a) comprises reacting the polysaccharide with 0.05-0.9 molar equivalents of periodate. In one embodiment step a) comprises reacting the polysaccharide with 0.1-0.9 molar equivalents of periodate. In one embodiment step a) comprises reacting the polysaccharide with 0.2-0.9 molar equivalents of periodate. In one embodiment step a) comprises reacting the polysaccharide with 0.3-0.9 molar equivalents of periodate. In one embodiment step a) comprises reacting the polysaccharide with 0.4-0.9 molar equivalents of periodate. In one embodiment step a) comprises reacting the polysaccharide with 0.5-0.9 molar equivalents of periodate. In one embodiment step a) comprises reacting the polysaccharide with 0.6-0.9 molar equivalents of periodate.

In one embodiment step a) comprises reacting the polysaccharide with 0.01-0.8 molar equivalents of periodate. In one embodiment step a) comprises reacting the polysaccharide with 0.05-0.8 molar equivalents of periodate. In one embodiment step a) comprises reacting the polysaccharide with 0.1-0.8 molar equivalents of periodate. In one embodiment step a) comprises reacting the polysaccharide with 0.2-0.8 molar equivalents of periodate. In one embodiment step a) comprises reacting the polysaccharide with 0.3-0.8 molar equivalents of periodate. In one embodiment step a) comprises reacting the polysaccharide with 0.4-0.8 molar equivalents of periodate. In one embodiment step a) comprises reacting the polysaccharide with 0.5-0.8 molar equivalents of periodate. In one embodiment step a) comprises reacting the polysaccharide with 0.6-0.8 molar equivalents of periodate.

In one embodiment step a) comprises reacting the polysaccharide with 0.01-0.7 molar equivalents of periodate. In one embodiment step a) comprises reacting the polysaccharide with 0.05-0.7 molar equivalents of periodate. In one embodiment step a) comprises reacting the polysaccharide with 0.1-0.7 molar equivalents of periodate. In one embodiment step a) comprises reacting the polysaccharide with 0.2-0.7 molar equivalents of periodate. In one embodiment step a) comprises reacting the polysaccharide with 0.3-0.7 molar equivalents of periodate. In one embodiment step a) comprises reacting the polysaccharide with 0.4-0.7 molar equivalents of periodate. In one embodiment step a) comprises reacting the polysaccharide with 0.5-0.7 molar equivalents of periodate. In one embodiment step a) comprises reacting the polysaccharide with 0.6-0.7 molar equivalents of periodate.

In one embodiment step a) comprises reacting the polysaccharide with 0.01-0.6 molar equivalents of periodate. In one embodiment step a) comprises reacting the polysaccharide with 0.05-0.6 molar equivalents of periodate. In one embodiment step a) comprises reacting the polysaccharide with 0.1-0.6 molar equivalents of periodate. In one embodiment step a) comprises reacting the polysaccharide with 0.2-0.6 molar equivalents of periodate. In one embodiment step a) comprises reacting the polysaccharide with 0.3-0.6 molar equivalents of periodate. In one embodiment step a) comprises reacting the polysaccharide with 0.4-0.6 molar equivalents of periodate. In one embodiment step a) comprises reacting the polysaccharide with 0.5-0.6 molar equivalents of periodate.

In one embodiment step a) comprises reacting the polysaccharide with 0.01-0.5 molar equivalents of periodate. In one embodiment step a) comprises reacting the polysaccharide with 0.05-0.5 molar equivalents of periodate. In one embodiment step a) comprises reacting the polysaccharide with 0.1-0.5 molar equivalents of periodate. In one embodiment step a) comprises reacting the polysaccharide with 0.2-0.5 molar equivalents of periodate. In one embodiment step a) comprises reacting the polysaccharide with 0.3-0.5 molar equivalents of periodate. In one embodiment step a) comprises reacting the polysaccharide with 0.4-0.5 molar equivalents of periodate.

In one embodiment step a) comprises reacting the polysaccharide with 0.01-0.4 molar equivalents of periodate. In one embodiment step a) comprises reacting the polysaccharide with 0.05-0.4 molar equivalents of periodate. In one embodiment step a) comprises reacting the polysaccharide with 0.1-0.4 molar equivalents of periodate. In one embodiment step a) comprises reacting the polysaccharide with 0.2-0.4 molar equivalents of periodate. In one embodiment step a) comprises reacting the polysaccharide with 0.3-0.4 molar equivalents of periodate.

In one embodiment step a) comprises reacting the polysaccharide with 0.01-0.3 molar equivalents of periodate. In one embodiment step a) comprises reacting the polysaccharide with 0.05-0.3 molar equivalents of periodate. In one embodiment step a) comprises reacting the polysaccharide with 0.1-0.3 molar equivalents of periodate. In one embodiment step a) comprises reacting the polysaccharide with 0.2-0.3 molar equivalents of periodate.

In one embodiment step a) comprises reacting the polysaccharide with 0.01-0.2 molar equivalents of periodate. In one embodiment step a) comprises reacting the polysaccharide with 0.05-0.2 molar equivalents of periodate. In one embodiment step a) comprises reacting the polysaccharide with 0.1-0.2 molar equivalents of periodate.

In one embodiment step a) comprises reacting the polysaccharide with 0.01-0.1 molar equivalents of periodate. In one embodiment step a) comprises reacting the polysaccharide with 0.05-0.1 molar equivalents of periodate.

In one embodiment, the quenching agent of step a′) is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

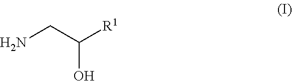

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

Preferably, the quenching agent is a compound of formula (II):

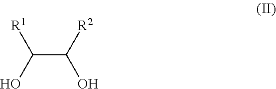

wherein R¹ and R² are each independently selected from H, methyl, ethyl, propyl or isopropyl.

In a preferred embodiment, the quenching agent is glycerol, ethylene glycol, propan-1,2-diol, butan-1,2-diol or butan-2,3-diol, or ascorbic acid. In an even preferred embodiment, the quenching agent is butan-2,3-diol.

In a preferred embodiment the degree of oxidation (also named “degree of activation” in the present document) of the activated serotype 3 polysaccharide is between 2 and 30. In an embodiment the degree of oxidation of the activated serotype 3 polysaccharide is between 2 and 25. In an embodiment the degree of oxidation of the activated serotype 3 polysaccharide is between 2 and 20. In an embodiment the degree of oxidation of the activated serotype 3 polysaccharide is between 2 and 15. In an embodiment the degree of oxidation of the activated serotype 3 polysaccharide is between 2 and 10. In an embodiment the degree of oxidation of the activated serotype 3 polysaccharide is between 2 and 5. In an embodiment the degree of oxidation of the activated serotype 3 polysaccharide is between 5 and 30. In an embodiment the degree of oxidation of the activated serotype 3 polysaccharide is between 5 and 25. In an embodiment the degree of oxidation of the activated serotype 3 polysaccharide is between 5 and 20. In an embodiment the degree of oxidation of the activated serotype 3 polysaccharide is between 5 and 15. In an embodiment the degree of oxidation of the activated serotype 3 polysaccharide is between 5 and 10. In an embodiment the degree of oxidation of the activated serotype 3 polysaccharide is between 10 and 30. In an embodiment the degree of oxidation of the activated serotype 3 polysaccharide is between 10 and 25. In an embodiment the degree of oxidation of the activated serotype 3 polysaccharide is between 10 and 20. In an embodiment the degree of oxidation of the activated serotype 3 polysaccharide is between 10 and 15. In a preferred embodiment the degree of oxidation of the activated serotype 3 polysaccharide is between 12 and 16. In an embodiment the degree of oxidation of the activated serotype 3 polysaccharide is between 15 and 30. In an embodiment the degree of oxidation of the activated serotype 3 polysaccharide is between 15 and 25. In an embodiment the degree of oxidation of the activated serotype 3 polysaccharide is between 15 and 20. In an embodiment the degree of oxidation of the activated serotype 3 polysaccharide is between 20 to 30. In an embodiment the degree of oxidation of the activated serotype 3 polysaccharide is between 20 to 25. In a preferred embodiment the degree of oxidation of the activated serotype 3 polysaccharide is between 10 to 20. In an even preferred embodiment the degree of oxidation of the activated serotype 3 polysaccharide is between 11 to 19

In a preferred embodiment the degree of oxidation of the activated serotype 3 polysaccharide is 15±4.

In an embodiment the degree of oxidation of the activated serotype 3 polysaccharide is about 5. In an embodiment the degree of oxidation of the activated serotype 3 polysaccharide is about 7. In an embodiment the degree of oxidation of the activated serotype 3 polysaccharide is about 10. In an embodiment the degree of oxidation of the activated serotype 3 polysaccharide is about 11. In an embodiment the degree of oxidation of the activated serotype 3 polysaccharide is about 12. In an embodiment the degree of oxidation of the activated serotype 3 polysaccharide is about 13. In a preferred embodiment the degree of oxidation of the activated serotype 3 polysaccharide is about 14. In an embodiment the degree of oxidation of the activated serotype 3 polysaccharide is about 15. In an embodiment the degree of oxidation of the activated serotype 3 polysaccharide is about 16. In an embodiment the degree of oxidation of the activated serotype 3 polysaccharide is about 17. In an embodiment the degree of oxidation of the activated serotype 3 polysaccharide is about 18. In an embodiment the degree of oxidation of the activated serotype 3 polysaccharide is about 19. In an embodiment the degree of oxidation of the activated serotype 3 polysaccharide is about 20. In a preferred embodiment the degree of oxidation of the activated serotype 3 polysaccharide is about 15.

In one embodiment the activated polysaccharide and the carrier protein are lyophilised before step b). Preferably lyophilisation occurs after step a). In one embodiment the activated polysaccharide is lyophilised after step a) and the carrier protein is also lyophilised.

In one embodiment the activated polysaccharide is lyophilised after step a) and the carrier protein is also lyophilised, and the activated polysaccharide and the carrier protein are reconstituted in the same solution, this acts as compounding the activated polysaccharide and the carrier protein together.

In an embodiment, the activated polysaccharide and the carrier protein are lyophilised independently (discrete lyophilization). In an embodiment, the activated polysaccharide and the carrier protein are lyophilised together (co-lyophilized).

In one embodiment the lyophilization takes place in the presence of a non-reducing sugar, possible non-reducing sugars include sucrose, trehalose, raffinose, stachyose, melezitose, dextran, mannitol, lactitol and palatinit. In an embodiment the sugar is selected from the group consisting of sucrose, trehalose, and mannitol. In an embodiment the sugar is sucrose, trehalose or mannitol. In an embodiment the sugar is trehalose. In an embodiment the sugar is sucrose.

In an embodiment the initial input ratio (weight by weight) of activated serotype 3 capsular polysaccharide to carrier protein at step b) is between 4:1 and 0.1:1. In an embodiment the initial input ratio (weight by weight) of activated serotype 3 capsular polysaccharide to carrier protein is between 2:1 and 0.4:1. In an embodiment the initial input ratio (weight by weight) of activated serotype 3 capsular polysaccharide to carrier protein is between 1.5:1 and 0.5:1. In a preferred embodiment the initial input ratio (weight by weight) of activated serotype 3 capsular polysaccharide to carrier protein is between 1.2:1 and 0.7:1. In an embodiment the initial input ratio (weight by weight) of activated serotype 3 capsular polysaccharide to carrier protein is about 4. In an embodiment the initial input ratio (weight by weight) of activated serotype 3 capsular polysaccharide to carrier protein is about 3. In an embodiment the initial input ratio (weight by weight) of activated serotype 3 capsular polysaccharide to carrier protein is about 2. In an embodiment the initial input ratio (weight by weight) of activated serotype 3 capsular polysaccharide to carrier protein is about 1.5.

In an embodiment the initial input ratio (weight by weight) of activated serotype 3 capsular polysaccharide to carrier protein is about 1.2. In an embodiment the initial input ratio (weight by weight) of activated serotype 3 capsular polysaccharide to carrier protein is about 1.1. In an embodiment the initial input ratio (weight by weight) of activated serotype 3 capsular polysaccharide to carrier protein is about 1.0. In an embodiment the initial input ratio (weight by weight) of activated serotype 3 capsular polysaccharide to carrier protein is about 0.9. In an embodiment the initial input ratio (weight by weight) of activated serotype 3 capsular polysaccharide to carrier protein is about 0.8. In an embodiment the initial input ratio (weight by weight) of activated serotype 3 capsular polysaccharide to carrier protein is about 0.7.

In an embodiment, the reduction reaction (c) is carried out in aqueous solvent. In an embodiment, the reduction reaction (c) is carried out in aprotic solvent.

In an embodiment, the reduction reaction (c) is carried out in the presence of dimethylsulphoxide (DMSO) or dimethylformamide (DMF). In an embodiment, the reduction reaction (c) is carried out in the presence of dimethylformamide (DMF). In an embodiment, the reduction reaction (c) is carried out in the presence of dimethylsulphoxide (DMSO).

In one embodiment the reduction reaction (c) is carried out in a solution consisting essentially of dimethylsulphoxide (DMSO) or dimethylformamide (DMF). In one embodiment the reduction reaction (c) is carried out in a solution consisting essentially of dimethylformamide (DMF). In one embodiment the reduction reaction (c) is carried out in a solution consisting essentially of dimethylsulphoxide (DMSO).

In an embodiment, the reduction reaction (c) is carried out in DMSO (dimethylsulfoxide) or in DMF (dimethylformamide)) solvent. In an embodiment, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMe′PrN-BH₃, benzylamine-BH₃ or 5-ethyl-2-methylpyridine borane (PEMB). In an embodiment, the reducing agent is sodium triacetoxyborohydride. In a preferred embodiment, the reducing agent is sodium cyanoborohydride. In an embodiment, the reducing agent is sodium cyanoborohydride in the present of nickel (see WO2018144439).

In one embodiment between 0.2 and 20 molar equivalents of reducing agent is used in step c). In one embodiment between 0.2 and 15 molar equivalents of reducing agent is used in step c). In one embodiment between 0.2 and 10 molar equivalents of reducing agent is used in step c). In one embodiment between 0.2 and 7 molar equivalents of reducing agent is used in step c). In one embodiment between 0.2 and 5 molar equivalents of reducing agent is used in step c). In one embodiment between 0.2 and 3 molar equivalents of reducing agent is used in step c). In one embodiment between 0.2 and 2 molar equivalents of reducing agent is used in step c). In one embodiment between 0.2 and 1.0 molar equivalent of reducing agent is used in step c). In one embodiment between 0.2 and 0.8 molar equivalent of reducing agent is used in step c).

In one embodiment between 0.5 and 20 molar equivalents of reducing agent is used in step c). In one embodiment between 0.5 and 15 molar equivalents of reducing agent is used in step c). In one embodiment between 0.5 and 10 molar equivalents of reducing agent is used in step c). In one embodiment between 0.5 and 7 molar equivalents of reducing agent is used in step c). In one embodiment between 0.5 and 5 molar equivalents of reducing agent is used in step c). In one embodiment between 0.5 and 3 molar equivalents of reducing agent is used in step c). In one embodiment between 0.5 and 2 molar equivalents of reducing agent is used in step c). In one embodiment between 0.5 and 1.0 molar equivalent of reducing agent is used in step c). In one embodiment between 0.5 and 0.8 molar equivalent of reducing agent is used in step c).

In one embodiment between 1.0 and 20 molar equivalents of reducing agent is used in step c). In one embodiment between 1.0 and 15 molar equivalents of reducing agent is used in step c). In one embodiment between 1.0 and 10 molar equivalents of reducing agent is used in step c). In one embodiment between 1.0 and 7 molar equivalents of reducing agent is used in step c). In one embodiment between 1.0 and 5 molar equivalents of reducing agent is used in step c). In one embodiment between 1.0 and 3 molar equivalents of reducing agent is used in step c). In one embodiment between 1.0 and 2 molar equivalents of reducing agent is used in step c).

In one embodiment between 1.5 and 20 molar equivalents of reducing agent is used in step c). In one embodiment between 1.5 and 15 molar equivalents of reducing agent is used in step c). In one embodiment between 1.5 and 10 molar equivalents of reducing agent is used in step c). In one embodiment between 1.5 and 7 molar equivalents of reducing agent is used in step c). In one embodiment between 1.5 and 5 molar equivalents of reducing agent is used in step c). In one embodiment between 1.5 and 3 molar equivalents of reducing agent is used in step c). In one embodiment between 1.5 and 2 molar equivalents of reducing agent is used in step c).

In one embodiment between 2 and 20 molar equivalents of reducing agent is used in step c). In one embodiment between 2 and 15 molar equivalents of reducing agent is used in step c). In one embodiment between 2 and 10 molar equivalents of reducing agent is used in step c). In one embodiment between 2 and 7 molar equivalents of reducing agent is used in step c). In one embodiment between 2 and 5 molar equivalents of reducing agent is used in step c). In one embodiment between 2 and 3 molar equivalents of reducing agent is used in step c).

In one embodiment between 3 and 20 molar equivalents of reducing agent is used in step c). In one embodiment between 3 and 15 molar equivalents of reducing agent is used in step c). In one embodiment between 3 and 10 molar equivalents of reducing agent is used in step c). In one embodiment between 3 and 7 molar equivalents of reducing agent is used in step c). In one embodiment between 3 and 5 molar equivalents of reducing agent is used in step c).

In one embodiment between 5 and 20 molar equivalents of reducing agent is used in step c). In one embodiment between 5 and 15 molar equivalents of reducing agent is used in step c). In one embodiment between 5 and 10 molar equivalents of reducing agent is used in step c). In one embodiment between 5 and 7 molar equivalents of reducing agent is used in step c).

In one embodiment between 7 and 20 molar equivalents of reducing agent is used in step c). In one embodiment between 7 and 15 molar equivalents of reducing agent is used in step c). In one embodiment between 7 and 10 molar equivalents of reducing agent is used in step c).

In one embodiment between 10 and 20 molar equivalents of reducing agent is used in step c). In one embodiment between 10 and 15 molar equivalents of reducing agent is used in step c).

In one embodiment between 15 and 20 molar equivalents of reducing agent is used in step c).

At the end of the reduction reaction, there may be unreacted aldehyde groups remaining in the conjugates, these may be capped using a suitable capping agent. In one embodiment this capping agent is sodium borohydride (NaBH₄).

In an embodiment, the product of step c) may be reacted with sodium borohydride for 15 mins-15 hrs. In an embodiment, the product of step c) may be reacted with sodium borohydride for 2-10 hrs. In an embodiment, the product of step c) may be reacted with sodium borohydride for 15 mins-1 hr. In an embodiment, the product of step c) may be reacted with sodium borohydride for around 4 hrs. In an embodiment, the product of step c) may be reacted with sodium borohydride for around 6 hrs. In an embodiment, the product of step c) may be reacted with sodium borohydride for around 2 hrs. In an embodiment, the product of step c) may be reacted with sodium borohydride for around 1 hr. In an embodiment, the product of step c) may be reacted with sodium borohydride for around 30 mins.

In an embodiment capping is achieved by mixing the product of step c) with 1 to 20 molar equivalents of sodium borohydride. In an embodiment capping is achieved by mixing the product of step c) with 1 to 15 molar equivalents of sodium borohydride. In an embodiment capping is achieved by mixing the product of step c) with 1 to 10 molar equivalents of sodium borohydride. In an embodiment capping is achieved by mixing the product of step c) with 1 to 7 molar equivalents of sodium borohydride. In an embodiment capping is achieved by mixing the product of step c) with 1 to 5 molar equivalents of sodium borohydride. In an embodiment capping is achieved by mixing the product of step c) with 1 to 3 molar equivalents of sodium borohydride. In an embodiment capping is achieved by mixing the product of step c) with 1 to 2 molar equivalents of sodium borohydride.

In an embodiment capping is achieved by mixing the product of step c) with 1.5 to 20 molar equivalents of sodium borohydride. In an embodiment capping is achieved by mixing the product of step c) with 1.5 to 15 molar equivalents of sodium borohydride. In an embodiment capping is achieved by mixing the product of step c) with 1.5 to 10 molar equivalents of sodium borohydride. In an embodiment capping is achieved by mixing the product of step c) with 1.5 to 7 molar equivalents of sodium borohydride. In an embodiment capping is achieved by mixing the product of step c) with 1.5 to 5 molar equivalents of sodium borohydride. In an embodiment capping is achieved by mixing the product of step c) with 1.5 to 3 molar equivalents of sodium borohydride. In an embodiment capping is achieved by mixing the product of step c) with 1.5 to 2 molar equivalents of sodium borohydride.

In an embodiment capping is achieved by mixing the product of step c) with 2 to 20 molar equivalents of sodium borohydride. In an embodiment capping is achieved by mixing the product of step c) with 2 to 15 molar equivalents of sodium borohydride. In an embodiment capping is achieved by mixing the product of step c) with 2 to 10 molar equivalents of sodium borohydride. In an embodiment capping is achieved by mixing the product of step c) with 2 to 7 molar equivalents of sodium borohydride. In an embodiment capping is achieved by mixing the product of step c) with 2 to 5 molar equivalents of sodium borohydride. In an embodiment capping is achieved by mixing the product of step c) with 2 to 3 molar equivalents of sodium borohydride.

In an embodiment capping is achieved by mixing the product of step c) with 3 to 20 molar equivalents of sodium borohydride. In an embodiment capping is achieved by mixing the product of step c) with 3 to 15 molar equivalents of sodium borohydride. In an embodiment capping is achieved by mixing the product of step c) with 3 to 10 molar equivalents of sodium borohydride. In an embodiment capping is achieved by mixing the product of step c) with 3 to 7 molar equivalents of sodium borohydride. In an embodiment capping is achieved by mixing the product of step c) with 3 to 5 molar equivalents of sodium borohydride.

In an embodiment capping is achieved by mixing the product of step c) with 5 to 20 molar equivalents of sodium borohydride. In an embodiment capping is achieved by mixing the product of step c) with 5 to 15 molar equivalents of sodium borohydride. In an embodiment capping is achieved by mixing the product of step c) with 5 to 10 molar equivalents of sodium borohydride. In an embodiment capping is achieved by mixing the product of step c) with 5 to 7 molar equivalents of sodium borohydride.

In an embodiment capping is achieved by mixing the product of step c) with 7 to 20 molar equivalents of sodium borohydride. In an embodiment capping is achieved by mixing the product of step c) with 7 to 15 molar equivalents of sodium borohydride. In an embodiment capping is achieved by mixing the product of step c) with 7 to 10 molar equivalents of sodium borohydride.

In an embodiment capping is achieved by mixing the product of step c) with 10 to 20 molar equivalents of sodium borohydride. In an embodiment capping is achieved by mixing the product of step c) with 10 to 15 molar equivalents of sodium borohydride.

In an embodiment capping is achieved by mixing the product of step c) with 15 to 20 molar equivalents of sodium borohydride.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

1.4 Streptococcus pneumoniae Serotype 3 Glycoconjugates of the Invention Prepared Using CDI and/or CDT Chemistry

In an embodiment, the glycoconjugate of the present invention is prepared using CDI and/or CDT chemistry.

The invention also relates to a method of making a serotype 3 glycoconjugate, as disclosed herein.

CDI and/or CDT chemistry involves two steps, (1) reacting the isolated saccharide with CDI and/or CDT in an aprotic solvent to produce an activated saccharide (activation), (2) reacting the activated saccharide with a carrier protein (e.g. CRM₁₉₇ or SCP) to form a glycoconjugate.

In an embodiment, the activating agent of step (1) is 1,1′-carbonyldiimidazole (CDI). In an embodiment, the activating agent of step (1) is 1,1′-Carbonyl-di-(1,2,4-triazole) (CDT).

As mentioned above, before activation with CDI and/or CDT, sizing of the polysaccharide to a target molecular weight (MW) range can be performed.

Therefore, in an embodiment, the isolated polysaccharide is sized before activation with CDI. In an embodiment, the isolated polysaccharide is sized before activation with CDT. In an embodiment, the isolated polysaccharide is sized to any of the target molecular weight (MW) range defined above.

In an embodiment, the isolated serotype 3 capsular polysaccharide is conjugated to a carrier protein by a process comprising the step of:

(a) reacting said isolated polysaccharide with CDI and/or CDT in an aprotic solvent;
(b) reacting the activated polysaccharide of step (a) with a carrier protein in an aprotic solvent to form a glycoconjugate.

Following step (a) the polysaccharide is said to be activated and is referred to as “activated polysaccharide”.

In an embodiment, the isolated serotype 3 capsular polysaccharide is conjugated to a carrier protein by a process comprising the step of:

(a) reacting said isolated polysaccharide with 1,1′-carbonyldiimidazole (CDI) in an aprotic solvent;
(b) reacting the activated polysaccharide of step (a) with a carrier protein in an aprotic solvent to form a glycoconjugate.

Following step (a) the polysaccharide is said to be activated and is referred to as “activated polysaccharide”.

In one embodiment step a) comprises reacting the polysaccharide with an amount of CDI that is between 0.01-10 molar equivalent to the amount of serotype 3 capsular polysaccharide present in the reaction mixture.

In one embodiment step a) comprises reacting the polysaccharide with an amount of CDI that is between 0.5-10 molar equivalent to the amount of serotype 3 capsular polysaccharide present in the reaction mixture.

In one embodiment step a) comprises reacting the polysaccharide with an amount of CDI that is between 0.1-10 molar equivalent to the amount of serotype 3 capsular polysaccharide present in the reaction mixture.

In one embodiment step a) comprises reacting the polysaccharide with an amount of CDI that is between 0.5-10 molar equivalent to the amount of serotype 3 capsular polysaccharide present in the reaction mixture.

In one embodiment step a) comprises reacting the polysaccharide with an amount of CDI that is between 1-10 molar equivalent to the amount of serotype 3 capsular polysaccharide present in the reaction mixture.

In one embodiment step a) comprises reacting the polysaccharide with an amount of CDI that is between 2-10 molar equivalent to the amount of serotype 3 capsular polysaccharide present in the reaction mixture.

In one embodiment step a) comprises reacting the polysaccharide with an amount of CDI that is between 3-10 molar equivalent to the amount of serotype 3 capsular polysaccharide present in the reaction mixture.

In one embodiment step a) comprises reacting the polysaccharide with an amount of CDI that is between 4-10 molar equivalent to the amount of serotype 3 capsular polysaccharide present in the reaction mixture.

In one embodiment step a) comprises reacting the polysaccharide with an amount of CDI that is between 5-10 molar equivalent to the amount of serotype 3 capsular polysaccharide present in the reaction mixture.

In one embodiment step a) comprises reacting the polysaccharide with an amount of CDI that is between 8-10 molar equivalent to the amount of serotype 3 capsular polysaccharide present in the reaction mixture.

In one embodiment step a) comprises reacting the polysaccharide with an amount of CDI that is between 0.01-8 molar equivalent to the amount of serotype 3 capsular polysaccharide present in the reaction mixture.

In one embodiment step a) comprises reacting the polysaccharide with an amount of CDI that is between 0.01-5 molar equivalent to the amount of serotype 3 capsular polysaccharide present in the reaction mixture.

In one embodiment step a) comprises reacting the polysaccharide with an amount of CDI that is between 0.01-4 molar equivalent to the amount of serotype 3 capsular polysaccharide present in the reaction mixture.

In one embodiment step a) comprises reacting the polysaccharide with an amount of CDI that is between 0.01-3 molar equivalent to the amount of serotype 3 capsular polysaccharide present in the reaction mixture.

In one embodiment step a) comprises reacting the polysaccharide with an amount of CDI that is between 0.01-2 molar equivalent to the amount of serotype 3 capsular polysaccharide present in the reaction mixture.

In one embodiment step a) comprises reacting the polysaccharide with an amount of CDI that is between 0.01-1 molar equivalent to the amount of serotype 3 capsular polysaccharide present in the reaction mixture.

In one embodiment step a) comprises reacting the polysaccharide with an amount of CDI that is between 0.01-0.5 molar equivalent to the amount of serotype 3 capsular polysaccharide present in the reaction mixture.

In one embodiment step a) comprises reacting the polysaccharide with an amount of CDI that is between 0.01-0.2 molar equivalent to the amount of serotype 3 capsular polysaccharide present in the reaction mixture.

In one embodiment step a) comprises reacting the polysaccharide with an amount of CDI that is between 0.01-0.1 molar equivalent to the amount of serotype 3 capsular polysaccharide present in the reaction mixture.

In one embodiment step a) comprises reacting the polysaccharide with an amount of CDI that is between 0.01-0.05 molar equivalent to the amount of serotype 3 capsular polysaccharide present in the reaction mixture.

In one embodiment step a) comprises reacting the polysaccharide with an amount of CDI that is between 0.05-8 molar equivalent to the amount of serotype 3 capsular polysaccharide present in the reaction mixture.

In one embodiment step a) comprises reacting the polysaccharide with an amount of CDI that is between 0.05-5 molar equivalent to the amount of serotype 3 capsular polysaccharide present in the reaction mixture.

In one embodiment step a) comprises reacting the polysaccharide with an amount of CDI that is between 0.05-4 molar equivalent to the amount of serotype 3 capsular polysaccharide present in the reaction mixture.

In one embodiment step a) comprises reacting the polysaccharide with an amount of CDI that is between 0.05-3 molar equivalent to the amount of serotype 3 capsular polysaccharide present in the reaction mixture.

In one embodiment step a) comprises reacting the polysaccharide with an amount of CDI that is between 0.05-2 molar equivalent to the amount of serotype 3 capsular polysaccharide present in the reaction mixture.

In one embodiment step a) comprises reacting the polysaccharide with an amount of CDI that is between 0.05-1 molar equivalent to the amount of serotype 3 capsular polysaccharide present in the reaction mixture.

In one embodiment step a) comprises reacting the polysaccharide with an amount of CDI that is between 0.05-0.5 molar equivalent to the amount of serotype 3 capsular polysaccharide present in the reaction mixture.

In one embodiment step a) comprises reacting the polysaccharide with an amount of CDI that is between 0.05-0.2 molar equivalent to the amount of serotype 3 capsular polysaccharide present in the reaction mixture.

In one embodiment step a) comprises reacting the polysaccharide with an amount of CDI that is between 0.05-0.1 molar equivalent to the amount of serotype 3 capsular polysaccharide present in the reaction mixture.

In one embodiment step a) comprises reacting the polysaccharide with an amount of CDI that is between 0.1-8 molar equivalent to the amount of serotype 3 capsular polysaccharide present in the reaction mixture.

In one embodiment step a) comprises reacting the polysaccharide with an amount of CDI that is between 0.1-5 molar equivalent to the amount of serotype 3 capsular polysaccharide present in the reaction mixture.

In one embodiment step a) comprises reacting the polysaccharide with an amount of CDI that is between 0.1-4 molar equivalent to the amount of serotype 3 capsular polysaccharide present in the reaction mixture.

In one embodiment step a) comprises reacting the polysaccharide with an amount of CDI that is between 0.1-3 molar equivalent to the amount of serotype 3 capsular polysaccharide present in the reaction mixture.

In one embodiment step a) comprises reacting the polysaccharide with an amount of CDI that is between 0.1-2 molar equivalent to the amount of serotype 3 capsular polysaccharide present in the reaction mixture.

In one embodiment step a) comprises reacting the polysaccharide with an amount of CDI that is between 0.1-1 molar equivalent to the amount of serotype 3 capsular polysaccharide present in the reaction mixture.

In one embodiment step a) comprises reacting the polysaccharide with an amount of CDI that is between 0.1-0.5 molar equivalent to the amount of serotype 3 capsular polysaccharide present in the reaction mixture.

In one embodiment step a) comprises reacting the polysaccharide with an amount of CDI that is between 0.5-5 molar equivalent to the amount of serotype 3 capsular polysaccharide present in the reaction mixture.

In one embodiment step a) comprises reacting the polysaccharide with an amount of CDI that is between 1-5 molar equivalent to the amount of serotype 3 capsular polysaccharide present in the reaction mixture.

In one embodiment step a) comprises reacting the polysaccharide with an amount of CDI that is between 2-5 molar equivalent to the amount of serotype 3 capsular polysaccharide present in the reaction mixture.

In one embodiment step a) comprises reacting the polysaccharide with an amount of CDI that is between 0.1-3 molar equivalent to the amount of serotype 3 capsular polysaccharide present in the reaction mixture.

In one embodiment step a) comprises reacting the polysaccharide with an amount of CDI that is between 0.5-3 molar equivalent to the amount of serotype 3 capsular polysaccharide present in the reaction mixture.

In one embodiment step a) comprises reacting the polysaccharide with an amount of CDI that is between 1-3 molar equivalent to the amount of serotype 3 capsular polysaccharide present in the reaction mixture.

In one embodiment step a) comprises reacting the polysaccharide with an amount of CDI that is between 1-2 molar equivalent to the amount of serotype 3 capsular polysaccharide present in the reaction mixture.

In one embodiment step a) comprises reacting the polysaccharide with an amount of CDI of about 0.01 molar equivalent to the amount of serotype 3 capsular polysaccharide present in the reaction mixture.

In one embodiment step a) comprises reacting the polysaccharide with an amount of CDI of about 0.05 molar equivalent to the amount of serotype 3 capsular polysaccharide present in the reaction mixture.

In one embodiment step a) comprises reacting the polysaccharide with an amount of CDI of about 0.1 molar equivalent to the amount of serotype 3 capsular polysaccharide present in the reaction mixture.

In one embodiment step a) comprises reacting the polysaccharide with an amount of CDI of about 0.5 molar equivalent to the amount of serotype 3 capsular polysaccharide present in the reaction mixture.

In one embodiment step a) comprises reacting the polysaccharide with an amount of CDI of about 1 molar equivalent to the amount of serotype 3 capsular polysaccharide present in the reaction mixture.

In one embodiment step a) comprises reacting the polysaccharide with an amount of CDI of about 2 molar equivalent to the amount of serotype 3 capsular polysaccharide present in the reaction mixture.

In one embodiment step a) comprises reacting the polysaccharide with an amount of CDI of about 3 molar equivalent to the amount of serotype 3 capsular polysaccharide present in the reaction mixture.

In one embodiment step a) comprises reacting the polysaccharide with an amount of CDI of about 4 molar equivalent to the amount of serotype 3 capsular polysaccharide present in the reaction mixture.

In one embodiment step a) comprises reacting the polysaccharide with an amount of CDI of about 5 molar equivalent to the amount of serotype 3 capsular polysaccharide present in the reaction mixture.

In one embodiment step a) comprises reacting the polysaccharide with an amount of CDI of about 8 molar equivalent to the amount of serotype 3 capsular polysaccharide present in the reaction mixture.

In one embodiment step a) comprises reacting the polysaccharide with an amount of CDI of about 10 molar equivalent to the amount of serotype 3 capsular polysaccharide present in the reaction mixture.

In an embodiment, the activating reaction a) is carried out in the presence of dimethylsulphoxide (DMSO), dimethylformamide (DMF), dimethylacetamide, N-methyl-2-pyrrolidone or hexamethylphosphoramide (HMPA). In an embodiment, the activating reaction a) is carried out in the presence of dimethylformamide (DMF). In an embodiment, the activating reaction a) is carried out in the presence of dimethylsulphoxide (DMSO). In an embodiment, the activating reaction a) is carried out in the presence of dimethylacetamide. In an embodiment, the activating reaction a) is carried out in the presence of N-methyl-2-pyrrolidone. In an embodiment, the activating reaction a) is carried out in the presence of hexamethylphosphoramide (HMPA).

In one embodiment the activating reaction a) is carried out in a solution consisting essentially of dimethylsulphoxide (DMSO) or dimethylformamide (DMF). In one embodiment the activating reaction a) is carried out in a solution consisting essentially of dimethylformamide (DMF). In one embodiment the activating reaction a) is carried out in a solution consisting essentially of dimethylsulphoxide (DMSO).

In an embodiment, the activating reaction a) is carried out in a solution consisting essentially of dimethylacetamide. In an embodiment, the activating reaction a) is carried out in a solution consisting essentially of N-methyl-2-pyrrolidone. In an embodiment, the activating reaction a) is carried out in a solution consisting essentially of hexamethylphosphoramide (HMPA).

It has been surprisingly found that conducting the activating reaction with a moisture level of about 0.1% to 1% allows to avoid side reactions. Therefore, in one embodiment the activating reaction a) is carried out in an aprotic solvent comprising 0.1% to 1% water. In one embodiment the activating reaction a) is carried out in an aprotic solvent comprising 0.1% to 0.8% water. In one embodiment the activating reaction a) is carried out in an aprotic solvent comprising 0.1% to 0.5% water. In one embodiment the activating reaction a) is carried out in an aprotic solvent comprising 0.1% to 0.4% water. In one embodiment the activating reaction a) is carried out in an aprotic solvent comprising 0.1% to 0.3% water. In one embodiment the activating reaction a) is carried out in an aprotic solvent comprising 0.1% to 0.2% water.

In one embodiment the activating reaction a) is carried out in an aprotic solvent comprising 0.2% to 1% water. In one embodiment the activating reaction a) is carried out in an aprotic solvent comprising 0.2% to 0.8% water. In one embodiment the activating reaction a) is carried out in an aprotic solvent comprising 0.2% to 0.5% water. In one embodiment the activating reaction a) is carried out in an aprotic solvent comprising 0.2% to 0.4% water. In one embodiment the activating reaction a) is carried out in an aprotic solvent comprising 0.2% to 0.3% water.

In one embodiment the activating reaction a) is carried out in an aprotic solvent comprising 0.3% to 0.8% water. In one embodiment the activating reaction a) is carried out in an aprotic solvent comprising 0.3% to 0.5% water. In one embodiment the activating reaction a) is carried out in an aprotic solvent comprising 0.3% to 0.4% water.

In one embodiment the activating reaction a) is carried out in an aprotic solvent comprising about 0.1% water. In one embodiment the activating reaction a) is carried out in an aprotic solvent comprising about 0.2% water. In one embodiment the activating reaction a) is carried out in an aprotic solvent comprising about 0.3% water. In one embodiment the activating reaction a) is carried out in an aprotic solvent comprising about 0.4% water. In one embodiment the activating reaction a) is carried out in an aprotic solvent comprising about 0.5% water. In one embodiment the activating reaction a) is carried out in an aprotic solvent comprising about 0.6% water. In one embodiment the activating reaction a) is carried out in an aprotic solvent comprising about 0.7% water. In one embodiment the activating reaction a) is carried out in an aprotic solvent comprising about 0.8% water. In one embodiment the activating reaction a) is carried out in an aprotic solvent comprising about 0.9% water.

In one embodiment the activating reaction a) is carried out in DMSO comprising 0.1% to 1% water. In one embodiment the activating reaction a) is carried out in DMSO comprising 0.1% to 0.8% water. In one embodiment the activating reaction a) is carried out in DMSO comprising 0.1% to 0.5% water. In one embodiment the activating reaction a) is carried out in DMSO comprising 0.1% to 0.4% water. In one embodiment the activating reaction a) is carried out in DMSO comprising 0.1% to 0.3% water. In one embodiment the activating reaction a) is carried out in DMSO comprising 0.1% to 0.2% water.

In one embodiment the activating reaction a) is carried out in DMSO comprising 0.2% to 1% water. In one embodiment the activating reaction a) is carried out in DMSO comprising 0.2% to 0.8% water. In one embodiment the activating reaction a) is carried out in DMSO comprising 0.2% to 0.5% water. In one embodiment the activating reaction a) is carried out in DMSO comprising 0.2% to 0.4% water. In one embodiment the activating reaction a) is carried out in DMSO comprising 0.2% to 0.3% water.

In one embodiment the activating reaction a) is carried out in DMSO comprising 0.3% to 0.8% water. In one embodiment the activating reaction a) is carried out in DMSO comprising 0.3% to 0.5% water. In one embodiment the activating reaction a) is carried out in DMSO comprising 0.3% to 0.4% water.

In one embodiment the activating reaction a) is carried out in DMSO comprising about 0.1% water. In one embodiment the activating reaction a) is carried out in DMSO comprising about 0.2% water. In one embodiment the activating reaction a) is carried out in DMSO comprising about 0.3% water. In one embodiment the activating reaction a) is carried out in DMSO comprising about 0.4% water. In one embodiment the activating reaction a) is carried out in DMSO comprising about 0.5% water. In one embodiment the activating reaction a) is carried out in DMSO comprising about 0.6% water. In one embodiment the activating reaction a) is carried out in DMSO comprising about 0.7% water. In one embodiment the activating reaction a) is carried out in DMSO comprising about 0.8% water. In one embodiment the activating reaction a) is carried out in DMSO comprising about 0.9% water.

In one embodiment the activating reaction a) is quenched by the addition of water. Water can inactivate free CDI.

Therefore, in an embodiment, the activating reaction a) is followed by the addition of water. In an embodiment, water is added to bring the total water content in the mixture to between about 1% to about 10% (v/v). In an embodiment, water is added to bring the total water content in the mixture to between about 1.2% to about 8% (v/v). In an embodiment, water is added to bring the total water content in the mixture to between about 1.5% to about 5% (v/v). In an embodiment, water is added to bring the total water content in the mixture to between about 1.5% to about 3% (v/v). In an embodiment, water is added to bring the total water content in the mixture to between about 1.5% to about 2.5% (v/v). In an embodiment, water is added to bring the total water content in the mixture to about 1% (v/v). In an embodiment, water is added to bring the total water content in the mixture to about 1.2% (v/v). In an embodiment, water is added to bring the total water content in the mixture to about 1.4% (v/v). In an embodiment, water is added to bring the total water content in the mixture to about 1.5% (v/v). In an embodiment, water is added to bring the total water content in the mixture to about 2% (v/v). In an embodiment, water is added to bring the total water content in the mixture to about 2.5% (v/v). In an embodiment, water is added to bring the total water content in the mixture to about 3% (v/v). In an embodiment, water is added to bring the total water content in the mixture to about 5% (v/v). In an embodiment, water is added to bring the total water content in the mixture to about 7% (v/v). In an embodiment, water is added to bring the total water content in the mixture to about 10% (v/v).

In an embodiment, the conjugation reaction b) is carried out in the presence of dimethylsulphoxide (DMSO), dimethylformamide (DMF), dimethylacetamide, N-methyl-2-pyrrolidone or hexamethylphosphoramide (HMPA).

In an embodiment, the conjugation reaction b) is carried out in the presence of dimethylformamide (DMF). In an embodiment, the conjugation reaction b) is carried out in the presence of dimethylsulphoxide (DMSO). In an embodiment, the conjugation reaction b) is carried out in the presence of dimethylacetamide. In an embodiment, the conjugation reaction b) is carried out in the presence of N-methyl-2-pyrrolidone. In an embodiment, the conjugation reaction b) is carried out in the presence of hexamethylphosphoramide (HMPA).

In one embodiment the conjugation reaction b) is carried out in a solution consisting essentially of dimethylsulphoxide (DMSO) or dimethylformamide (DMF). In one embodiment the conjugation reaction b) is carried out in a solution consisting essentially of dimethylformamide (DMF). In one embodiment the conjugation reaction b) is carried out in a solution consisting essentially of dimethylsulphoxide (DMSO).

In one embodiment the conjugation reaction b) is carried out in a solution consisting essentially of dimethylacetamide. In one embodiment the conjugation reaction b) is carried out in a solution consisting essentially of N-methyl-2-pyrrolidone. In one embodiment the conjugation reaction b) is carried out in a solution consisting essentially of hexamethylphosphoramide (HMPA).

In one embodiment the conjugation reaction b) is carried out in DMSO comprising about 0.1% to about 10% v/v water. In one embodiment the conjugation reaction b) is carried out in DMSO comprising about 0.1% to about 8% v/v water. In one embodiment the conjugation reaction b) is carried out in DMSO comprising about 0.1% to about 5% v/v water. In one embodiment the conjugation reaction b) is carried out in DMSO comprising about 0.1% to about 2% v/v water. In one embodiment the conjugation reaction b) is carried out in DMSO comprising about 0.1% to about 1% v/v water. In one embodiment the conjugation reaction b) is carried out in DMSO comprising about 1.5% to about 2.5% v/v water. In one embodiment the conjugation reaction b) is carried out in DMSO comprising about 1.5% to about 3% v/v water. In one embodiment the conjugation reaction b) is carried out in DMSO comprising about 0.1% v/v water. In one embodiment the conjugation reaction b) is carried out in DMSO comprising about 0.5% v/v water. In one embodiment the conjugation reaction b) is carried out in DMSO comprising about 1% v/v water. In one embodiment the conjugation reaction b) is carried out in DMSO comprising about 2% v/v water. In one embodiment the conjugation reaction b) is carried out in DMSO comprising about 3% v/v water. In one embodiment the conjugation reaction b) is carried out in DMSO comprising about 4% v/v water. In one embodiment the conjugation reaction b) is carried out in DMSO comprising about 5% v/v water. In one embodiment the conjugation reaction b) is carried out in DMSO comprising about 6% v/v water. In one embodiment the conjugation reaction b) is carried out in DMSO comprising about 8% v/v water. In one embodiment the conjugation reaction b) is carried out in DMSO comprising about 10% v/v water.

In an embodiment the initial input ratio (weight by weight) of activated serotype 3 capsular polysaccharide to carrier protein at step b) is between 4:1 and 0.1:1. In an embodiment the initial input ratio (weight by weight) of activated serotype 3 capsular polysaccharide to carrier protein is between 2:1 and 0.4:1. In an embodiment the initial input ratio (weight by weight) of activated serotype 3 capsular polysaccharide to carrier protein is between 1.5:1 and 0.5:1. In a preferred embodiment the initial input ratio (weight by weight) of activated serotype 3 capsular polysaccharide to carrier protein is between 1.2:1 and 0.7:1. In an embodiment the initial input ratio (weight by weight) of activated serotype 3 capsular polysaccharide to carrier protein is about 4. In an embodiment the initial input ratio (weight by weight) of activated serotype 3 capsular polysaccharide to carrier protein is about 3. In an embodiment the initial input ratio (weight by weight) of activated serotype 3 capsular polysaccharide to carrier protein is about 2. In an embodiment the initial input ratio (weight by weight) of activated serotype 3 capsular polysaccharide to carrier protein is about 1.5. In an embodiment the initial input ratio (weight by weight) of activated serotype 3 capsular polysaccharide to carrier protein is about 1.2. In an embodiment the initial input ratio (weight by weight) of activated serotype 3 capsular polysaccharide to carrier protein is about 1.1. In an embodiment the initial input ratio (weight by weight) of activated serotype 3 capsular polysaccharide to carrier protein is about 1.0. In an embodiment the initial input ratio (weight by weight) of activated serotype 3 capsular polysaccharide to carrier protein is about 0.9. In an embodiment the initial input ratio (weight by weight) of activated serotype 3 capsular polysaccharide to carrier protein is about 0.8. In an embodiment the initial input ratio (weight by weight) of activated serotype 3 capsular polysaccharide to carrier protein is about 0.7.

In one embodiment, weak organic base can be added to the reaction mixture after the activating reaction a) but before the conjugation reaction b). The weak organic base can be added before or after the carrier protein is introduced the reaction mixture. Therefore, in one embodiment, the weak organic base is added to the reaction mixture before the carrier protein is introduced. In another embodiment, the weak organic base is added to the reaction mixture after the carrier protein is introduced. Weak organic base can be selected from alkanamines, imidazole, triazole, pyridine, histidine and guanidine. Alkanamines include alkyl primary amines such as methyl amine, ethylamine, propylamine, isopropylamine; alkyl secondary amines such as dimethyl amine, diethylamine, dipropylamine, diisopropylamine; alkyl tertially amines such as trimethyl amine, triethylamine, tri-isopropylamine, di-N,N′-isopropylethylamine, et al. In an embodiment, the weak organic base is an alkanamine. In an embodiment, the weak organic base is an imidazole. In an embodiment, the weak organic base is a triazole. In an embodiment, the weak organic base is pyridine. In an embodiment, the weak organic base is histidine. In an embodiment, the weak organic base is guanidine.

In one embodiment, the organic base is added with an amount between 0.1-25 times (weight by weight) of the amount of polysaccharide to be activated. In one embodiment the ratio can be 0.5-20 times (weight by weight), In one embodiment the ratio can be 1-10 times (weight by weight), in another embodiment the ratio can be 1-5 times (weight by weight).

In one embodiment following the conjugation reaction b) unconjugated reactive sites of the activated polysaccharide are hydrolyzed. In one embodiment unconjugated reactive sites are hydrolyzed by addition to the conjugation solution of an aqueous solution. In one embodiment unconjugated reactive sites are hydrolyzed by addition to the conjugation solution of an aqueous buffered solution. In one embodiment unconjugated reactive sites are hydrolyzed by addition to the conjugation solution of an aqueous buffered solution and adjustment of the pH to between about 3.0 to about 10.0. In one embodiment unconjugated reactive sites are hydrolyzed by addition to the conjugation solution of an aqueous buffered solution and adjustment of the pH to between about 7.0 to about 10.0. In one embodiment unconjugated reactive sites are hydrolyzed by addition to the conjugation solution of an aqueous buffered solution and adjustment of the pH to between about 3.0 to about 7.0. In one embodiment unconjugated reactive sites are hydrolyzed by addition to the conjugation solution of an aqueous buffered solution and adjustment of the pH to about 4.0. In one embodiment unconjugated reactive sites are hydrolyzed by addition to the conjugation solution of an aqueous buffered solution and adjustment of the pH to about 9.0.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

In an embodiment, the isolated serotype 3 capsular polysaccharide is conjugated to a carrier protein by a process comprising the step of:

(a) reacting said isolated polysaccharide with 1,1′-Carbonyl-di-(1,2,4-triazole) (CDT) in an aprotic solvent;
(b) reacting the activated polysaccharide of step (a) with a carrier protein in an aprotic solvent to form a glycoconjugate.

Following step (a) the polysaccharide is said to be activated and is referred to as “activated polysaccharide”.

In one embodiment step a) comprises reacting the polysaccharide with an amount of CDT that is between 0.01-10 molar equivalent to the amount of serotype 3 capsular polysaccharide present in the reaction mixture.

In one embodiment step a) comprises reacting the polysaccharide with an amount of CDT that is between 0.5-10 molar equivalent to the amount of serotype 3 capsular polysaccharide present in the reaction mixture.

In one embodiment step a) comprises reacting the polysaccharide with an amount of CDT that is between 0.1-10 molar equivalent to the amount of serotype 3 capsular polysaccharide present in the reaction mixture.

In one embodiment step a) comprises reacting the polysaccharide with an amount of CDT that is between 0.5-10 molar equivalent to the amount of serotype 3 capsular polysaccharide present in the reaction mixture.

In one embodiment step a) comprises reacting the polysaccharide with an amount of CDT that is between 1-10 molar equivalent to the amount of serotype 3 capsular polysaccharide present in the reaction mixture.

In one embodiment step a) comprises reacting the polysaccharide with an amount of CDT that is between 2-10 molar equivalent to the amount of serotype 3 capsular polysaccharide present in the reaction mixture.

In one embodiment step a) comprises reacting the polysaccharide with an amount of CDT that is between 3-10 molar equivalent to the amount of serotype 3 capsular polysaccharide present in the reaction mixture.

In one embodiment step a) comprises reacting the polysaccharide with an amount of CDT that is between 4-10 molar equivalent to the amount of serotype 3 capsular polysaccharide present in the reaction mixture.

In one embodiment step a) comprises reacting the polysaccharide with an amount of CDT that is between 5-10 molar equivalent to the amount of serotype 3 capsular polysaccharide present in the reaction mixture.

In one embodiment step a) comprises reacting the polysaccharide with an amount of CDT that is between 8-10 molar equivalent to the amount of serotype 3 capsular polysaccharide present in the reaction mixture.

In one embodiment step a) comprises reacting the polysaccharide with an amount of CDT that is between 0.01-8 molar equivalent to the amount of serotype 3 capsular polysaccharide present in the reaction mixture.

In one embodiment step a) comprises reacting the polysaccharide with an amount of CDT that is between 0.01-5 molar equivalent to the amount of serotype 3 capsular polysaccharide present in the reaction mixture.

In one embodiment step a) comprises reacting the polysaccharide with an amount of CDT that is between 0.01-4 molar equivalent to the amount of serotype 3 capsular polysaccharide present in the reaction mixture.

In one embodiment step a) comprises reacting the polysaccharide with an amount of CDT that is between 0.01-3 molar equivalent to the amount of serotype 3 capsular polysaccharide present in the reaction mixture.

In one embodiment step a) comprises reacting the polysaccharide with an amount of CDT that is between 0.01-2 molar equivalent to the amount of serotype 3 capsular polysaccharide present in the reaction mixture.

In one embodiment step a) comprises reacting the polysaccharide with an amount of CDT that is between 0.01-1 molar equivalent to the amount of serotype 3 capsular polysaccharide present in the reaction mixture.

In one embodiment step a) comprises reacting the polysaccharide with an amount of CDT that is between 0.01-0.5 molar equivalent to the amount of serotype 3 capsular polysaccharide present in the reaction mixture.

In one embodiment step a) comprises reacting the polysaccharide with an amount of CDT that is between 0.01-0.2 molar equivalent to the amount of serotype 3 capsular polysaccharide present in the reaction mixture.

In one embodiment step a) comprises reacting the polysaccharide with an amount of CDT that is between 0.01-0.1 molar equivalent to the amount of serotype 3 capsular polysaccharide present in the reaction mixture.

In one embodiment step a) comprises reacting the polysaccharide with an amount of CDT that is between 0.01-0.05 molar equivalent to the amount of serotype 3 capsular polysaccharide present in the reaction mixture.

In one embodiment step a) comprises reacting the polysaccharide with an amount of CDT that is between 0.05-8 molar equivalent to the amount of serotype 3 capsular polysaccharide present in the reaction mixture.

In one embodiment step a) comprises reacting the polysaccharide with an amount of CDT that is between 0.05-5 molar equivalent to the amount of serotype 3 capsular polysaccharide present in the reaction mixture.

In one embodiment step a) comprises reacting the polysaccharide with an amount of CDT that is between 0.05-4 molar equivalent to the amount of serotype 3 capsular polysaccharide present in the reaction mixture.

In one embodiment step a) comprises reacting the polysaccharide with an amount of CDT that is between 0.05-3 molar equivalent to the amount of serotype 3 capsular polysaccharide present in the reaction mixture.

In one embodiment step a) comprises reacting the polysaccharide with an amount of CDT that is between 0.05-2 molar equivalent to the amount of serotype 3 capsular polysaccharide present in the reaction mixture.

In one embodiment step a) comprises reacting the polysaccharide with an amount of CDT that is between 0.05-1 molar equivalent to the amount of serotype 3 capsular polysaccharide present in the reaction mixture.

In one embodiment step a) comprises reacting the polysaccharide with an amount of CDT that is between 0.05-0.5 molar equivalent to the amount of serotype 3 capsular polysaccharide present in the reaction mixture.

In one embodiment step a) comprises reacting the polysaccharide with an amount of CDT that is between 0.05-0.2 molar equivalent to the amount of serotype 3 capsular polysaccharide present in the reaction mixture.

In one embodiment step a) comprises reacting the polysaccharide with an amount of CDT that is between 0.05-0.1 molar equivalent to the amount of serotype 3 capsular polysaccharide present in the reaction mixture.

In one embodiment step a) comprises reacting the polysaccharide with an amount of CDT that is between 0.1-8 molar equivalent to the amount of serotype 3 capsular polysaccharide present in the reaction mixture.

In one embodiment step a) comprises reacting the polysaccharide with an amount of CDT that is between 0.1-5 molar equivalent to the amount of serotype 3 capsular polysaccharide present in the reaction mixture.

In one embodiment step a) comprises reacting the polysaccharide with an amount of CDT that is between 0.1-4 molar equivalent to the amount of serotype 3 capsular polysaccharide present in the reaction mixture.

In one embodiment step a) comprises reacting the polysaccharide with an amount of CDT that is between 0.1-3 molar equivalent to the amount of serotype 3 capsular polysaccharide present in the reaction mixture.

In one embodiment step a) comprises reacting the polysaccharide with an amount of CDT that is between 0.1-2 molar equivalent to the amount of serotype 3 capsular polysaccharide present in the reaction mixture.

In one embodiment step a) comprises reacting the polysaccharide with an amount of CDT that is between 0.1-1 molar equivalent to the amount of serotype 3 capsular polysaccharide present in the reaction mixture.

In one embodiment step a) comprises reacting the polysaccharide with an amount of CDT that is between 0.1-0.5 molar equivalent to the amount of serotype 3 capsular polysaccharide present in the reaction mixture.

In one embodiment step a) comprises reacting the polysaccharide with an amount of CDT that is between 0.5-5 molar equivalent to the amount of serotype 3 capsular polysaccharide present in the reaction mixture.

In one embodiment step a) comprises reacting the polysaccharide with an amount of CDT that is between 1-5 molar equivalent to the amount of serotype 3 capsular polysaccharide present in the reaction mixture.

In one embodiment step a) comprises reacting the polysaccharide with an amount of CDT that is between 2-5 molar equivalent to the amount of serotype 3 capsular polysaccharide present in the reaction mixture.

In one embodiment step a) comprises reacting the polysaccharide with an amount of CDT that is between 0.1-3 molar equivalent to the amount of serotype 3 capsular polysaccharide present in the reaction mixture.

In one embodiment step a) comprises reacting the polysaccharide with an amount of CDT that is between 0.5-3 molar equivalent to the amount of serotype 3 capsular polysaccharide present in the reaction mixture.

In one embodiment step a) comprises reacting the polysaccharide with an amount of CDT that is between 1-3 molar equivalent to the amount of serotype 3 capsular polysaccharide present in the reaction mixture.

In one embodiment step a) comprises reacting the polysaccharide with an amount of CDT that is between 1-2 molar equivalent to the amount of serotype 3 capsular polysaccharide present in the reaction mixture.

In one embodiment step a) comprises reacting the polysaccharide with an amount of CDT of about 0.01 molar equivalent to the amount of serotype 3 capsular polysaccharide present in the reaction mixture.

In one embodiment step a) comprises reacting the polysaccharide with an amount of CDT of about 0.05 molar equivalent to the amount of serotype 3 capsular polysaccharide present in the reaction mixture.

In one embodiment step a) comprises reacting the polysaccharide with an amount of CDT of about 0.1 molar equivalent to the amount of serotype 3 capsular polysaccharide present in the reaction mixture.

In one embodiment step a) comprises reacting the polysaccharide with an amount of CDT of about 0.5 molar equivalent to the amount of serotype 3 capsular polysaccharide present in the reaction mixture.

In one embodiment step a) comprises reacting the polysaccharide with an amount of CDT of about 1 molar equivalent to the amount of serotype 3 capsular polysaccharide present in the reaction mixture.

In one embodiment step a) comprises reacting the polysaccharide with an amount of CDT of about 2 molar equivalent to the amount of serotype 3 capsular polysaccharide present in the reaction mixture.

In one embodiment step a) comprises reacting the polysaccharide with an amount of CDT of about 3 molar equivalent to the amount of serotype 3 capsular polysaccharide present in the reaction mixture.

In one embodiment step a) comprises reacting the polysaccharide with an amount of CDT of about 4 molar equivalent to the amount of serotype 3 capsular polysaccharide present in the reaction mixture.

In one embodiment step a) comprises reacting the polysaccharide with an amount of CDT of about 5 molar equivalent to the amount of serotype 3 capsular polysaccharide present in the reaction mixture.

In one embodiment step a) comprises reacting the polysaccharide with an amount of CDT of about 8 molar equivalent to the amount of serotype 3 capsular polysaccharide present in the reaction mixture.

In one embodiment step a) comprises reacting the polysaccharide with an amount of CDT of about 10 molar equivalent to the amount of serotype 3 capsular polysaccharide present in the reaction mixture.

In an embodiment, the activating reaction a) is carried out in the presence of dimethylsulphoxide (DMSO), dimethylformamide (DMF), dimethylacetamide, N-methyl-2-pyrrolidone or hexamethylphosphoramide (HMPA). In an embodiment, the activating reaction a) is carried out in the presence of dimethylformamide (DMF). In an embodiment, the activating reaction a) is carried out in the presence of dimethylsulphoxide (DMSO). In an embodiment, the activating reaction a) is carried out in the presence of dimethylacetamide. In an embodiment, the activating reaction a) is carried out in the presence of N-methyl-2-pyrrolidone. In an embodiment, the activating reaction a) is carried out in the presence of hexamethylphosphoramide (HMPA).

In one embodiment the activating reaction a) is carried out in a solution consisting essentially of dimethylsulphoxide (DMSO) or dimethylformamide (DMF). In one embodiment the activating reaction a) is carried out in a solution consisting essentially of dimethylformamide (DMF). In one embodiment the activating reaction a) is carried out in a solution consisting essentially of dimethylsulphoxide (DMSO).

In an embodiment, the activating reaction a) is carried out in a solution consisting essentially of dimethylacetamide. In an embodiment, the activating reaction a) is carried out in a solution consisting essentially of N-methyl-2-pyrrolidone. In an embodiment, the activating reaction a) is carried out in a solution consisting essentially of hexamethylphosphoramide (HMPA).

It has been surprisingly found that conducting the activating reaction with a moisture level of about 0.1% to 1% allows to avoid side reactions. Therefore, in one embodiment the activating reaction a) is carried out in an aprotic solvent comprising 0.1% to 1% water. In one embodiment the activating reaction a) is carried out in an aprotic solvent comprising 0.1% to 0.8% water. In one embodiment the activating reaction a) is carried out in an aprotic solvent comprising 0.1% to 0.5% water. In one embodiment the activating reaction a) is carried out in an aprotic solvent comprising 0.1% to 0.4% water. In one embodiment the activating reaction a) is carried out in an aprotic solvent comprising 0.1% to 0.3% water. In one embodiment the activating reaction a) is carried out in an aprotic solvent comprising 0.1% to 0.2% water.

In one embodiment the activating reaction a) is carried out in an aprotic solvent comprising 0.2% to 1% water. In one embodiment the activating reaction a) is carried out in an aprotic solvent comprising 0.2% to 0.8% water. In one embodiment the activating reaction a) is carried out in an aprotic solvent comprising 0.2% to 0.5% water. In one embodiment the activating reaction a) is carried out in an aprotic solvent comprising 0.2% to 0.4% water. In one embodiment the activating reaction a) is carried out in an aprotic solvent comprising 0.2% to 0.3% water.

In one embodiment the activating reaction a) is carried out in an aprotic solvent comprising 0.3% to 0.8% water. In one embodiment the activating reaction a) is carried out in an aprotic solvent comprising 0.3% to 0.5% water. In one embodiment the activating reaction a) is carried out in an aprotic solvent comprising 0.3% to 0.4% water.

In one embodiment the activating reaction a) is carried out in an aprotic solvent comprising about 0.1% water. In one embodiment the activating reaction a) is carried out in an aprotic solvent comprising about 0.2% water. In one embodiment the activating reaction a) is carried out in an aprotic solvent comprising about 0.3% water. In one embodiment the activating reaction a) is carried out in an aprotic solvent comprising about 0.4% water. In one embodiment the activating reaction a) is carried out in an aprotic solvent comprising about 0.5% water. In one embodiment the activating reaction a) is carried out in an aprotic solvent comprising about 0.6% water. In one embodiment the activating reaction a) is carried out in an aprotic solvent comprising about 0.7% water. In one embodiment the activating reaction a) is carried out in an aprotic solvent comprising about 0.8% water. In one embodiment the activating reaction a) is carried out in an aprotic solvent comprising about 0.9% water.

In one embodiment the activating reaction a) is carried out in DMSO comprising 0.1% to 1% water. In one embodiment the activating reaction a) is carried out in DMSO comprising 0.1% to 0.8% water. In one embodiment the activating reaction a) is carried out in DMSO comprising 0.1% to 0.5% water. In one embodiment the activating reaction a) is carried out in DMSO comprising 0.1% to 0.4% water. In one embodiment the activating reaction a) is carried out in DMSO comprising 0.1% to 0.3% water. In one embodiment the activating reaction a) is carried out in DMSO comprising 0.1% to 0.2% water.

In one embodiment the activating reaction a) is carried out in DMSO comprising 0.2% to 1% water. In one embodiment the activating reaction a) is carried out in DMSO comprising 0.2% to 0.8% water. In one embodiment the activating reaction a) is carried out in DMSO comprising 0.2% to 0.5% water. In one embodiment the activating reaction a) is carried out in DMSO comprising 0.2% to 0.4% water. In one embodiment the activating reaction a) is carried out in DMSO comprising 0.2% to 0.3% water.

In one embodiment the activating reaction a) is carried out in DMSO comprising 0.3% to 0.8% water. In one embodiment the activating reaction a) is carried out in DMSO comprising 0.3% to 0.5% water. In one embodiment the activating reaction a) is carried out in DMSO comprising 0.3% to 0.4% water.

In one embodiment the activating reaction a) is carried out in DMSO comprising about 0.1% water. In one embodiment the activating reaction a) is carried out in DMSO comprising about 0.2% water. In one embodiment the activating reaction a) is carried out in DMSO comprising about 0.3% water. In one embodiment the activating reaction a) is carried out in DMSO comprising about 0.4% water. In one embodiment the activating reaction a) is carried out in DMSO comprising about 0.5% water. In one embodiment the activating reaction a) is carried out in DMSO comprising about 0.6% water. In one embodiment the activating reaction a) is carried out in DMSO comprising about 0.7% water. In one embodiment the activating reaction a) is carried out in DMSO comprising about 0.8% water. In one embodiment the activating reaction a) is carried out in DMSO comprising about 0.9% water.

In one embodiment the activating reaction a) is quenched by the addition of water. Water can inactivate free CDT.

Therefore, in an embodiment, the activating reaction a) is followed by the addition of water. In an embodiment, water is added to bring the total water content in the mixture to between about 1% to about 10% (v/v). In an embodiment, water is added to bring the total water content in the mixture to between about 1.2% to about 8% (v/v). In an embodiment, water is added to bring the total water content in the mixture to between about 1.5% to about 5% (v/v). In an embodiment, water is added to bring the total water content in the mixture to between about 1.5% to about 3% (v/v). In an embodiment, water is added to bring the total water content in the mixture to between about 1.5% to about 2.5% (v/v). In an embodiment, water is added to bring the total water content in the mixture to about 1% (v/v). In an embodiment, water is added to bring the total water content in the mixture to about 1.2% (v/v). In an embodiment, water is added to bring the total water content in the mixture to about 1.4% (v/v). In an embodiment, water is added to bring the total water content in the mixture to about 1.5% (v/v). In an embodiment, water is added to bring the total water content in the mixture to about 2% (v/v). In an embodiment, water is added to bring the total water content in the mixture to about 2.5% (v/v). In an embodiment, water is added to bring the total water content in the mixture to about 3% (v/v). In an embodiment, water is added to bring the total water content in the mixture to about 5% (v/v). In an embodiment, water is added to bring the total water content in the mixture to about 7% (v/v). In an embodiment, water is added to bring the total water content in the mixture to about 10% (v/v).

In an embodiment, the conjugation reaction b) is carried out in the presence of dimethylsulphoxide (DMSO), dimethylformamide (DMF), dimethylacetamide, N-methyl-2-pyrrolidone or hexamethylphosphoramide (HMPA).

In an embodiment, the conjugation reaction b) is carried out in the presence of dimethylformamide (DMF). In an embodiment, the conjugation reaction b) is carried out in the presence of dimethylsulphoxide (DMSO). In an embodiment, the conjugation reaction b) is carried out in the presence of dimethylacetamide. In an embodiment, the conjugation reaction b) is carried out in the presence of N-methyl-2-pyrrolidone. In an embodiment, the conjugation reaction b) is carried out in the presence of hexamethylphosphoramide (HMPA).

In one embodiment the conjugation reaction b) is carried out in a solution consisting essentially of dimethylsulphoxide (DMSO) or dimethylformamide (DMF). In one embodiment the conjugation reaction b) is carried out in a solution consisting essentially of dimethylformamide (DMF). In one embodiment the conjugation reaction b) is carried out in a solution consisting essentially of dimethylsulphoxide (DMSO).

In one embodiment the conjugation reaction b) is carried out in a solution consisting essentially of dimethylacetamide. In one embodiment the conjugation reaction b) is carried out in a solution consisting essentially of N-methyl-2-pyrrolidone. In one embodiment the conjugation reaction b) is carried out in a solution consisting essentially of hexamethylphosphoramide (HMPA).

In one embodiment the conjugation reaction b) is carried out in DMSO comprising about 0.1% to about 10% v/v water. In one embodiment the conjugation reaction b) is carried out in DMSO comprising about 0.1% to about 8% v/v water. In one embodiment the conjugation reaction b) is carried out in DMSO comprising about 0.1% to about 5% v/v water. In one embodiment the conjugation reaction b) is carried out in DMSO comprising about 0.1% to about 2% v/v water. In one embodiment the conjugation reaction b) is carried out in DMSO comprising about 0.1% to about 1% v/v water. In one embodiment the conjugation reaction b) is carried out in DMSO comprising about 1.5% to about 2.5% v/v water. In one embodiment the conjugation reaction b) is carried out in DMSO comprising about 1.5% to about 3% v/v water. In one embodiment the conjugation reaction b) is carried out in DMSO comprising about 0.1% v/v water. In one embodiment the conjugation reaction b) is carried out in DMSO comprising about 0.5% v/v water. In one embodiment the conjugation reaction b) is carried out in DMSO comprising about 1% v/v water. In one embodiment the conjugation reaction b) is carried out in DMSO comprising about 2% v/v water. In one embodiment the conjugation reaction b) is carried out in DMSO comprising about 3% v/v water. In one embodiment the conjugation reaction b) is carried out in DMSO comprising about 4% v/v water. In one embodiment the conjugation reaction b) is carried out in DMSO comprising about 5% v/v water. In one embodiment the conjugation reaction b) is carried out in DMSO comprising about 6% v/v water. In one embodiment the conjugation reaction b) is carried out in DMSO comprising about 8% v/v water. In one embodiment the conjugation reaction b) is carried out in DMSO comprising about 10% v/v water.

In an embodiment the initial input ratio (weight by weight) of activated serotype 3 capsular polysaccharide to carrier protein at step b) is between 4:1 and 0.1:1. In an embodiment the initial input ratio (weight by weight) of activated serotype 3 capsular polysaccharide to carrier protein is between 2:1 and 0.4:1. In an embodiment the initial input ratio (weight by weight) of activated serotype 3 capsular polysaccharide to carrier protein is between 1.5:1 and 0.5:1. In a preferred embodiment the initial input ratio (weight by weight) of activated serotype 3 capsular polysaccharide to carrier protein is between 1.2:1 and 0.7:1. In an embodiment the initial input ratio (weight by weight) of activated serotype 3 capsular polysaccharide to carrier protein is about 4. In an embodiment the initial input ratio (weight by weight) of activated serotype 3 capsular polysaccharide to carrier protein is about 3. In an embodiment the initial input ratio (weight by weight) of activated serotype 3 capsular polysaccharide to carrier protein is about 2. In an embodiment the initial input ratio (weight by weight) of activated serotype 3 capsular polysaccharide to carrier protein is about 1.5. In an embodiment the initial input ratio (weight by weight) of activated serotype 3 capsular polysaccharide to carrier protein is about 1.2. In an embodiment the initial input ratio (weight by weight) of activated serotype 3 capsular polysaccharide to carrier protein is about 1.1. In an embodiment the initial input ratio (weight by weight) of activated serotype 3 capsular polysaccharide to carrier protein is about 1.0. In an embodiment the initial input ratio (weight by weight) of activated serotype 3 capsular polysaccharide to carrier protein is about 0.9. In an embodiment the initial input ratio (weight by weight) of activated serotype 3 capsular polysaccharide to carrier protein is about 0.8. In an embodiment the initial input ratio (weight by weight) of activated serotype 3 capsular polysaccharide to carrier protein is about 0.7.

In one embodiment, weak organic base can be added to the reaction mixture after the activating reaction a) but before the conjugation reaction b). The weak organic base can be added before or after the carrier protein is introduced the reaction mixture. Therefore, in one embodiment, the weak organic base is added to the reaction mixture before the carrier protein is introduced. In another embodiment, the weak organic base is added to the reaction mixture after the carrier protein is introduced. Weak organic base can be selected from alkanamines, imidazole, triazole, pyridine, histidine and guanidine. Alkanamines include alkyl primary amines such as methyl amine, ethylamine, propylamine, isopropylamine; alkyl secondary amines such as dimethyl amine, diethylamine, dipropylamine, diisopropylamine; alkyl tertially amines such as trimethyl amine, triethylamine, tri-isopropylamine, di-N,N′-isopropylethylamine, et al. In an embodiment, the weak organic base is an alkanamine. In an embodiment, the weak organic base is an imidazole. In an embodiment, the weak organic base is a triazole. In an embodiment, the weak organic base is pyridine. In an embodiment, the weak organic base is histidine. In an embodiment, the weak organic base is guanidine.

In one embodiment, the organic base is added with an amount between 0.1-25 times (weight by weight) of the amount of polysaccharide to be activated. In one embodiment the ratio can be 0.5-20 times (weight by weight), In one embodiment the ratio can be 1-10 times (weight by weight), in another embodiment the ratio can be 1-5 times (weight by weight).

In one embodiment following the conjugation reaction b) unconjugated reactive sites of the activated polysaccharide are hydrolyzed. In one embodiment unconjugated reactive sites are hydrolyzed by addition to the conjugation solution of an aqueous solution. In one embodiment unconjugated reactive sites are hydrolyzed by addition to the conjugation solution of an aqueous buffered solution. In one embodiment unconjugated reactive sites are hydrolyzed by addition to the conjugation solution of an aqueous buffered solution and adjustment of the pH to between about 3.0 to about 10.0. In one embodiment unconjugated reactive sites are hydrolyzed by addition to the conjugation solution of an aqueous buffered solution and adjustment of the pH to between about 7.0 to about 10.0. In one embodiment unconjugated reactive sites are hydrolyzed by addition to the conjugation solution of an aqueous buffered solution and adjustment of the pH to between about 3.0 to about 7.0. In one embodiment unconjugated reactive sites are hydrolyzed by addition to the conjugation solution of an aqueous buffered solution and adjustment of the pH to about 4.0. In one embodiment unconjugated reactive sites are hydrolyzed by addition to the conjugation solution of an aqueous buffered solution and adjustment of the pH to about 9.0.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

1.5 Carrier Protein of the Streptococcus pneumoniae Serotype 3 Glycoconjugates of the Invention

A component of the glycoconjugate is a carrier protein to which the purified polysaccharide is conjugated. The terms “protein carrier” or “carrier protein” or “carrier” may be used interchangeably herein. Carrier proteins should be amenable to standard conjugation procedures.

In a preferred embodiment, the carrier protein of the serotype 3 capsular polysaccharide glycoconjugate is selected in the group consisting of: DT (Diphtheria toxoid), TT (tetanus toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇, and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein ID, see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP).

In a preferred embodiment, the carrier protein of the serotype 3 capsular polysaccharide glycoconjugate of the invention is selected from the group consisting of TT, DT, DT mutants (such as CRM₁₉₇), and a C5a peptidase from Streptococcus (SCP).

In a preferred embodiment, the carrier protein of the serotype 3 capsular polysaccharide glycoconjugate of the invention is TT, DT, DT mutants (such as CRM₁₉₇) or a C5a peptidase from Streptococcus (SCP).

In an embodiment, the carrier protein of the serotype 3 capsular polysaccharide glycoconjugate is DT (Diphtheria toxoid). In another embodiment, the carrier protein of the serotype 3 capsular polysaccharide glycoconjugate is TT (tetanus toxoid).

In another embodiment, the carrier protein of the serotype 3 capsular polysaccharide glycoconjugate is PD (H. influenzae protein ID, see, e.g., EP0594610 B).

In a preferred embodiment, the carrier protein of the serotype 3 capsular polysaccharide glycoconjugate is CRM₁₉₇ or a C5a peptidase from Streptococcus (SCP).

In a preferred embodiment, the serotype 3 capsular polysaccharide is conjugated to CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{lox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the serotype 3 capsular polysaccharide is conjugated to CRM₁₉₇ protein or the A chain of CRM₁₉₇ (see CN103495161). In an embodiment, the serotype 3 capsular polysaccharide is conjugated the A chain of CRM₁₉₇ obtained via expression by genetically recombinant E. coli (see CN103495161).

In other preferred embodiments, the carrier protein of the serotype 3 capsular polysaccharide glycoconjugate of the invention is SCP (Streptococcal C5a Peptidase). Two important species of β-hemolytic streptococci, Streptococcus pyogenes (group A Streptococcus, GAS) and Streptococcus agalactiae (group B Streptococcus, GBS), which cause a variety of serious human infections that range from mild cases of pharyngitis and impetigo to serious invasive diseases such as necrotizing fasciitis (GAS) and neonatal sepsis (GBS) have developed a way to defeat this immune response. All human isolates of β-hemolytic streptococci, including GAS and GBS, produce a highly conserved cell-wall protein SCP (Streptococcal C5a Peptidase) that specifically inactivates C5a. The scp genes from GAS and GBS encode a polypeptide containing between 1,134 and 1,181 amino acids (Brown et al., PNAS, 2005, vol. 102, no. 51 pages 18391-18396). The first 31 residues are the export signal presequence and are removed upon passing through the cytoplasmic membrane. The next 68 residues serve as a pro-sequence and must be removed to produce active SCP. The next 10 residues can be removed without loss of protease activity. At the other end, starting with Lys-1034, are four consecutive 17-residue motifs followed by a cell sorting and cell-wall attachment signal. This combined signal is composed of a 20-residue hydrophilic sequence containing an LPTTND sequence, a 17-residue hydrophobic sequence, and a short basic carboxyl terminus.

SCP can be divided in domains (see FIG. 1B of Brown et al., PNAS, 2005, vol. 102, no. 51 pages 18391-18396). These domains are the Pre/Pro domain (which comprises the export signal presequence (commonly the first 31 residues) and the pro-sequence (commonly the next 68 residues)), the protease domain (which is splitted in two part (protease part 1 commonly residues 89-333/334 and protease domain part 2 and commonly residues 467/468-583/584), the protease-associated domain (PA domain) (commonly residues 333/334-467/468), three fibronectin type III (Fn) domains (Fn1, commonly residues 583/584-712/713; Fn2, commonly residues 712/713-928/929/930; commonly Fn3, residues 929/930-1029/1030/1031) and a cell wall anchor domain (commonly redisues 1029/1030/1031 to the C-terminus).

In an embodiment, the carrier protein of the serotype 3 capsular polysaccharide glycoconjugate of the invention is an SCP from GBS (SCPB). An exemple of SCPB is provided at SEQ. ID.NO: 3 of WO97/26008. See also SEQ ID NO: 3 of WO00/34487. In another preferred embodiments, the carrier protein of the serotype 3 capsular polysaccharide glycoconjugate of the invention is an SCP from GAS (SCPA).

Examples of SCPA can be found at SEQ.ID.No.1 and SEQ.ID.No.2 of WO97/26008. See also SEQ ID NO: 1, 2 and 23 of WO00/34487.

In a preferred embodiment, the carrier protein of the serotype 3 capsular polysaccharide glycoconjugate of the invention is an enzymatically inactive SCP.

In other preferred embodiments, the carrier protein of the serotype 3 capsular polysaccharide glycoconjugate of the invention is an enzymatically inactive SCP from GBS (SCPB).

In another preferred embodiments, the carrier protein of the serotype 3 capsular polysaccharide glycoconjugate of the invention is an enzymatically inactive SCP from GAS (SCPA).

In an embodiment, the carrier protein of the serotype 3 capsular polysaccharide glycoconjugate of the invention is a fragment of an SCP. In an embodiment, the carrier protein of the serotype 3 capsular polysaccharide glycoconjugate of the invention is a fragment of an SCPA. Preferably, the carrier protein of the serotype 3 capsular polysaccharide glycoconjugate of the invention is a fragment of an SCPB.

In an embodiment, the carrier protein of the serotype 3 capsular polysaccharide glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 3 capsular polysaccharide glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 3 capsular polysaccharide glycoconjugate of the invention is an enzymatically inactive fragment of an SCP which comprises the protease domain, the protease-associated domain (PA domain) and two of the three fibronectin type III (Fn) domains but does not comprise the export signal presequence, the pro-sequence and the cell wall anchor domain.

In an embodiment, the carrier protein of the serotype 3 capsular polysaccharide glycoconjugate of the invention is an enzymatically inactive fragment of an SCP. In an embodiment, said enzymatically inactive fragment of SCP comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 3 capsular polysaccharide glycoconjugate of the invention is an enzymatically inactive fragment of an SCPA. In an embodiment, said enzymatically inactive fragment of an SCPA comprises the protease domain, the protease-associated domain (PA domain) and the three fibronectin type III

(Fn) domains but does not comprise the export signal presequence, the pro-sequence and the cell wall anchor domain.

In a preferred embodiment, the carrier protein of the serotype 3 capsular polysaccharide glycoconjugate of the invention is an enzymatically inactive fragment of SCPB. Preferably, said enzymatically inactive fragment of SCPB comprises the protease domain, the protease-associated domain (PA domain) and the three fibronectin type III (Fn) domains but does not comprise the export signal presequence, the pro-sequence and the cell wall anchor domain.

In an embodiment, the enzymatic activity of SCP is inactivated by replacing at least one amino acid of the wild type sequence. In an embodiment, said replacement is selected from the group consisting of D130A, H193A, N295A and S512A. The numbers indicate the amino acid residue position in the peptidase according to the numbering of SEQ ID NO: 1 of WO00/34487.

Therefore, in an embodiment, the carrier protein of the serotype 3 capsular polysaccharide glycoconjugate of the invention is an enzymatically inactive SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 3 capsular polysaccharide glycoconjugate of the invention is an enzymatically inactive SCPA where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 3 capsular polysaccharide glycoconjugate of the invention is an enzymatically inactive SCPB where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 3 capsular polysaccharide glycoconjugate of the invention is an enzymatically inactive fragment of an SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 3 capsular polysaccharide glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 3 capsular polysaccharide glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 3 capsular polysaccharide glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the enzymatic activity of SCP is inactivated by replacing at least two amino acids of the wild type sequence. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In an embodiment, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

Therefore, in an embodiment, the carrier protein of the serotype 3 capsular polysaccharide glycoconjugate of the invention is an enzymatically inactive SCP where said inactivation is accomplished by replacing at least two amino acids of the wild type sequence. Preferably, said replacement of at least two amino acids is in the protease domain. In an embodiment, said replacement of at least two amino acid is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 3 capsular polysaccharide glycoconjugate of the invention is an enzymatically inactive SCPA where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 3 capsular polysaccharide glycoconjugate of the invention is an enzymatically inactive SCPB where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 3 capsular polysaccharide glycoconjugate of the invention is an enzymatically inactive fragment of an SCP where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 3 capsular polysaccharide glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 3 capsular polysaccharide glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least one amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 3 capsular polysaccharide glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the enzymatic activity of SCP is inactivated by replacing at least three amino acids of the wild type sequence. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

Therefore, in an embodiment, the carrier protein of the serotype 3 capsular polysaccharide glycoconjugate of the invention is an enzymatically inactive SCP where said inactivation is accomplished by replacing at least three amino acids of the wild type sequence. Preferably, said replacement of at least three amino acids is in the protease domain. In an embodiment, said replacement of at least three amino acid is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 3 capsular polysaccharide glycoconjugate of the invention is an enzymatically inactive SCPA where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 3 capsular polysaccharide glycoconjugate of the invention is an enzymatically inactive SCPB where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 3 capsular polysaccharide glycoconjugate of the invention is an enzymatically inactive fragment of an SCP where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 3 capsular polysaccharide glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 3 capsular polysaccharide glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 3 capsular polysaccharide glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the enzymatic activity of SCP is inactivated by replacing at least four amino acids of the wild type sequence. In an embodiment, said at least four amino acids replacements are D130A, H193A, N295A and S512A.

Therefore, in an embodiment, the carrier protein of the serotype 3 capsular polysaccharide glycoconjugate of the invention is an enzymatically inactive SCP where said inactivation is accomplished by replacing at least four amino acids of the wild type sequence. Preferably, said replacement of at least four amino acids is in the protease domain. In an embodiment, said replacement of at least four amino acid is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 3 capsular polysaccharide glycoconjugate of the invention is an enzymatically inactive SCPA where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 3 capsular polysaccharide glycoconjugate of the invention is an enzymatically inactive SCPB where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 3 capsular polysaccharide glycoconjugate of the invention is an enzymatically inactive fragment of an SCP where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 3 capsular polysaccharide glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 3 capsular polysaccharide glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least one amino acids is in part 2 of the protease domain. In an embodiment, said at least four amino acids replacements are D130A, H193A, N295A and S512A

In an embodiment, the carrier protein of the serotype 3 capsular polysaccharide glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acids is in part 2 of the protease domain. In an embodiment, said at least four amino acids replacements are D130A, H193A, N295A and S512A

In a particular embodiment, the carrier protein of the serotype 3 capsular polysaccharide glycoconjugate of the invention is an enzymatically inactive fragment of SCP which consists of SEQ ID NO: 41.

In a particular embodiment, the carrier protein of the serotype 3 capsular polysaccharide glycoconjugate of the invention is an enzymatically inactive fragment of SCP which consists of SEQ ID NO: 42.

SEQ ID NO: 41:
MAKTADTPATSKATIRDLNDPSQVKTLQEKAGKGA
GTVVAVIAAGFDKNHEAWRLTDKAKARYQSKEDLE
KAKKEHGITYGEWVNDKVAYYHDYSKDGKTAVDQE
HGTHVSGILSGNAPSETKEPYRLEGAMPEAQLLLM
RVEIVNGLADYARNYAQAIRDAINLGAKVINMSFG
NAALAYANLPDETKKAFDYAKSKGVSIVTSAGNDS
SFGGKTRLPLADHPDYGVVGTPAAADSTLTVASYS
PDKQLTETVTVKTADQQDKEMPVLSTNRFEPNKAY
DYAYANRGTKEDDFKDVKGKIALIERGDIDFKDKI
AKAKKAGAVGVLIYDNQDKGFPIELPNVDQMPAAF
ISRKDGLLLKDNPQKTITFNATPKVLPTASGTKLS
RFSSWGLTADGNIKPDIAAPGQDILSSVANNKYAK
LSGTAMSAPLVAGIMGLLQEQYETQYPDMTPSERL
DLAKKVLMSSATALYDEDEKAYFSPRQQGAGAVDA
KKASAATMYVTDKDNTSSKVHLNNVSDKFEVTVTV
HNKSDKPQELYYQATVQTDKVDGKHFALAPKALYE
TSWQKITIPANSSKQVTVPIDASRFSKDLLAQMKN
GYFLEGFVRFKQDPKKEELMSIPYIGFRGDFGNLS
ALEKPIYDSKDGSSYYHEANSDAKDQLDGDGLQFY
ALKNNFTALTTESNPWTIIKAVKEGVENIEDIESS
EITETIFAGTFAKQDDDSHYYIHRHANGKPYAAIS
PNGDGNRDYVQFQGTFLRNAKNLVAEVLDKEGNVV
WTSEVTEQVVKNYNNDLASTLGSTRFEKTRWDGKD
KDGKVVANGTYTYRVRYTPISSGAKEQHTDFDVIV
DNTTPEVATSATFSTEDRRLTLASKPKTSQPVYRE
RIAYTYMDEDLPTTEYISPNEDGTFTLPEEAETME
GATVPLKMSDFTYWEDMAGNITYTPVTKLLEGHSN
KPEQ
SEQ ID NO: 41 is 950 amino acids long.
SEQ ID NO: 42:
AKTADTPATSKATIRDLNDPSQVKTLQEKAGKGAG
TVVAVIAAGFDKNHEAWRLTDKAKARYQSKEDLEK
AKKEHGITYGEWVNDKVAYYHDYSKDGKTAVDQEH
GTHVSGILSGNAPSETKEPYRLEGAMPEAQLLLMR
VEIVNGLADYARNYAQAIRDAINLGAKVINMSFGN
AALAYANLPDETKKAFDYAKSKGVSIVTSAGNDSS
FGGKTRLPLADHPDYGVVGTPAAADSTLTVASYSP
DKQLTETVTVKTADQQDKEMPVLSTNRFEPNKAYD
YAYANRGTKEDDFKDVKGKIALIERGDIDFKDKIA
KAKKAGAVGVLIYDNQDKGFPIELPNVDQMPAAFI
SRKDGLLLKDNPQKTITFNATPKVLPTASGTKLSR
FSSWGLTADGNIKPDIAAPGQDILSSVANNKYAKL
SGTAMSAPLVAGIMGLLQEQYETQYPDMTPSERLD
LAKKVLMSSATALYDEDEKAYFSPRQQGAGAVDAK
KASAATMYVTDKDNTSSKVHLNNVSDKFEVTVTVH
NKSDKPQELYYQATVQTDKVDGKHFALAPKALYET
SWQKITIPANSSKQVTVPIDASRFSKDLLAQMKNG
YFLEGFVRFKQDPKKEELMSIPYIGFRGDFGNLSA
LEKPIYDSKDGSSYYHEANSDAKDQLDGDGLQFYA
LKNNFTALTTESNPWTIIKAVKEGVENIEDIESSE
ITETIFAGTFAKQDDDSHYYIHRHANGKPYAAISP
NGDGNRDYVQFQGTFLRNAKNLVAEVLDKEGNVVW
TSEVTEQVVKNYNNDLASTLGSTRFEKTRWDGKDK
DGKVVANGTYTYRVRYTPISSGAKEQHTDFDVIVD
NTTPEVATSATFSTEDRRLTLASKPKTSQPVYRER
IAYTYMDEDLPTTEYISPNEDGTFTLPEEAETMEG
ATVPLKMSDFTYWEDMAGNITYTPVTKLLEGHSNK
PEQ
SEQ ID NO: 42 is 949 amino acids long.

In a particular embodiment, the carrier protein of the serotype 3 capsular polysaccharide glycoconjugate of the invention is an enzymatically inactive fragment of SCP consisting of a polypeptide having at least 90% identity with SEQ ID NO: 41.

In a particular embodiment, the carrier protein of the serotype 3 capsular polysaccharide glycoconjugate of the invention is an enzymatically inactive fragment of SCP consisting of a polypeptide having at least 95% identity with SEQ ID NO: 41.

In a particular embodiment, the carrier protein of the serotype 3 capsular polysaccharide glycoconjugate of the invention is an enzymatically inactive fragment of SCP consisting of a polypeptide having at least 99% identity with SEQ ID NO: 41.

In a particular embodiment, the carrier protein of the serotype 3 capsular polysaccharide glycoconjugate of the invention is an enzymatically inactive fragment of SCP consisting of a polypeptide having at least 99.5% identity with SEQ ID NO: 41.

In a particular embodiment, the carrier protein of the serotype 3 capsular polysaccharide glycoconjugate of the invention is an enzymatically inactive fragment of SCP consisting of a polypeptide having at least 99.8% identity with SEQ ID NO: 41.

In a particular embodiment, the carrier protein of the serotype 3 capsular polysaccharide glycoconjugate of the invention is an enzymatically inactive fragment of SCP consisting of a polypeptide having at least 99.85% identity with SEQ ID NO: 41.

In a particular embodiment, the carrier protein of the serotype 3 capsular polysaccharide glycoconjugate of the invention is an enzymatically inactive fragment of SCP consisting of a polypeptide having at least 90% identity with SEQ ID NO: 42.

In a particular embodiment, the carrier protein of the serotype 3 capsular polysaccharide glycoconjugate of the invention is an enzymatically inactive fragment of SCP consisting of a polypeptide having at least 95% identity with SEQ ID NO: 42.

In a particular embodiment, the carrier protein of the serotype 3 capsular polysaccharide glycoconjugate of the invention is an enzymatically inactive fragment of SCP consisting of a polypeptide having at least 99% identity with SEQ ID NO: 42.

In a particular embodiment, the carrier protein of the serotype 3 capsular polysaccharide glycoconjugate of the invention is an enzymatically inactive fragment of SCP consisting of a polypeptide having at least 99.5% identity with SEQ ID NO: 42.

In a particular embodiment, the carrier protein of the serotype 3 capsular polysaccharide glycoconjugate of the invention is an enzymatically inactive fragment of SCP consisting of a polypeptide having at least 99.8% identity with SEQ ID NO: 42.

In a particular embodiment, the carrier protein of the serotype 3 capsular polysaccharide glycoconjugate of the invention is an enzymatically inactive fragment of SCP consisting of a polypeptide having at least 99.85% identity with SEQ ID NO: 42.

2 Immunogenic Compositions

2.1 Combinations of Glycoconjugates of the Invention

In an embodiment the invention relates to an immunogenic composition comprising a Streptococcus pneumoniae serotype 3 glycoconjugate of the invention.

In an embodiment the invention relates to an immunogenic composition comprising a Streptococcus pneumoniae serotype 3 glycoconjugate of the invention and comprising from 1 to 25 different glycoconjugates.

In an embodiment the invention relates to an immunogenic composition comprising a Streptococcus pneumoniae serotype 3 glycoconjugate of the invention and comprising from 1 to 25 glycoconjugates from different serotypes of S. pneumoniae (1 to 25 pneumococcal conjugates). In one embodiment the invention relates to an immunogenic composition comprising glycoconjugates from 7, 8, 9, 10, 11, 12, 13, 14, 15, 16, 17, 18, 19, 20, 21, 22, 23, 24 or 25 different serotypes of S. pneumoniae. In one embodiment the immunogenic composition comprises glycoconjugates from 16 or 20 different serotypes of S. pneumoniae. In an embodiment the immunogenic composition is a 7, 8, 9, 10, 11, 12, 13, 14, 15, 16, 17, 18, 19 or 20-valent pneumococcal conjugate compositions. In an embodiment the immunogenic composition is a 14, 15, 16, 17, 18 or 19-valent pneumococcal conjugate compositions. In an embodiment the immunogenic composition is a 16-valent pneumococcal conjugate composition. In an embodiment the immunogenic composition is a 19-valent pneumococcal conjugate composition. In an embodiment the immunogenic composition is a 20-valent pneumococcal conjugate composition.

In an embodiment the immunogenic composition is a 21, 22, 23, 24 or 25-valent pneumococcal conjugate compositions. In an embodiment the immunogenic composition is a 21-valent pneumococcal conjugate composition. In an embodiment the immunogenic composition is a 22-valent pneumococcal conjugate composition. In an embodiment the immunogenic composition is a 23-valent pneumococcal conjugate composition. In an embodiment the immunogenic composition is a 24-valent pneumococcal conjugate composition. In an embodiment the immunogenic composition is a 25-valent pneumococcal conjugate composition.

In an embodiment the invention relates to an immunogenic composition comprising a Streptococcus pneumoniae serotype 3 glycoconjugate of the invention and further comprising glycoconjugates from S. pneumoniae serotypes 4, 6B, 9V, 14, 18C, 19F and 23F.

In an embodiment said immunogenic composition comprises in addition glycoconjugates from S. pneumoniae serotypes 1, 5 and 7F.

In an embodiment any of the immunogenic compositions above comprises in addition glycoconjugates from S. pneumoniae serotypes 6A and 19A.

In an embodiment any of the immunogenic compositions above comprise in addition a glycoconjugates from S. pneumoniae serotype 22F and 33F.

In an embodiment any of the immunogenic compositions above comprise in addition a glycoconjugates from S. pneumoniae serotypes 8, 10A, 11A, 12F and 15B.

In an embodiment any of the immunogenic compositions above comprise in addition a glycoconjugates from S. pneumoniae serotype 2.

In an embodiment any of the immunogenic compositions above comprise in addition a glycoconjugates from S. pneumoniae serotypes 9N.

In an embodiment any of the immunogenic compositions above comprise in addition a glycoconjugates from S. pneumoniae serotypes 17F.

In an embodiment any of the immunogenic compositions above comprise in addition a glycoconjugates from S. pneumoniae serotypes 20.

In an embodiment the invention relates to an immunogenic composition comprising a Streptococcus pneumoniae serotype 3 glycoconjugate of the invention and further comprising glycoconjugates from S. pneumoniae serotypes 4, 6B, 9V, 14, 18C, 19F and 23F. In an embodiment the immunogenic composition is an 8-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6B, 7F, 9V, 14, 18C, 19F and 23F. In an embodiment the immunogenic composition is an 11-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 9V, 14, 18C, 19A, 19F and 23F. In an embodiment the immunogenic composition is a 13-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 9V, 14, 18C, 19A, 19F, 22F, 23F and 33F. In an embodiment the immunogenic composition is a 15-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F. In an embodiment the immunogenic composition is a 20-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 2, 4, 5, 6A, 6B, 7F, 8, 9V, 10A, 11A, 12F, 14, 15B, 18C, 19A, 19F, 22F, 23F and 33F. In an embodiment the immunogenic composition is a 21-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 9N, 10A, 11A, 12F, 14, 15B, 18C, 19A, 19F, 22F, 23F and 33F. In an embodiment the immunogenic composition is a 21-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 2, 4, 5, 6A, 6B, 7F, 8, 9V, 9N, 10A, 11A, 12F, 14, 15B, 18C, 19A, 19F, 22F, 23F and 33F. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 2, 4, 5, 6A, 6B, 7F, 8, 9V, 9N, 10A, 11A, 12F, 14, 15B, 17F, 18C, 19A, 19F, 22F, 23F and 33F. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 2, 4, 5, 6A, 6B, 7F, 8, 9V, 9N, 10A, 11A, 12F, 14, 15B, 17F, 18C, 19A, 19F, 20, 22F, 23F and 33F. In an embodiment the immunogenic composition is a 24-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23F and 33F. In an embodiment the immunogenic composition is a 21-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23A, 23F and 33F. In an embodiment the immunogenic composition is a 21-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23B, 23F and 33F. In an embodiment the immunogenic composition is a 21-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23F, 24F and 33F. In an embodiment the immunogenic composition is a 21-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23F, 33F and 35B. In an embodiment the immunogenic composition is a 21-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23F and 33F. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23B, 23F and 33F. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23F, 24F and 33F. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23F, 33F and 35B. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23A, 23B, 23F and 33F. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23A, 23F, 24F and 33F. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23A, 23F, 33F and 35B. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23B, 23F, 24F and 33F. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23B, 23F, 33F and 35B. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23F, 24F, 33F and 35B. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F and 33F. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23F, 24F and 33F. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23F, 33F and 35B. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23B, 23F, 24F and 33F. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23B, 23F, 33F and 35B. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23F, 24F, 33F and 35B. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23A, 23B, 23F, 33F and 35B. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23A, 23B, 23F, 24F and 33F. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23A, 23F, 24F, 33F and 35B. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23B, 23F, 24F, 33F and 35B. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F and 33F. In an embodiment the immunogenic composition is a 24-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 33F and 35B. In an embodiment the immunogenic composition is a 24-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23F, 24F, 33F and 35B. In an embodiment the immunogenic composition is a 24-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23B, 23F, 24F, 33F and 35B. In an embodiment the immunogenic composition is a 24-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15B, 18C, 19A, 19F, 22F, 23A, 23B, 23F, 24F, 33F and 35B. In an embodiment the immunogenic composition is a 24-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In an embodiment the immunogenic composition is a 25-valent pneumococcal conjugate compositions.

In an embodiment the invention relates to an immunogenic composition comprising a Streptococcus pneumoniae serotype 3 glycoconjugate of the invention and further comprising at least one glycoconjugate selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment the invention relates to an immunogenic composition comprising a Streptococcus pneumoniae serotype 3 glycoconjugate of the invention and further comprising twenty glycoconjugates selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 21-valent pneumococcal conjugate compositions.

In an embodiment the invention relates to an immunogenic composition comprising a Streptococcus pneumoniae serotype 3 glycoconjugate of the invention and further comprising twenty one glycoconjugates selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 22-valent pneumococcal conjugate compositions.

In an embodiment the invention relates to an immunogenic composition comprising a Streptococcus pneumoniae serotype 3 glycoconjugate of the invention and further comprising at least one glycoconjugate selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38.

In an embodiment the invention relates to an immunogenic composition comprising a Streptococcus pneumoniae serotype 3 glycoconjugate of the invention and further comprising twenty two glycoconjugates selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising twenty three glycoconjugates selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 24-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 2, 9N, 15A, 17F, 20, 23A, 23B, 24F and 35B. In an embodiment the immunogenic composition is a 10-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 2, 9N, 15A, 17F, 19A, 19F, 20, 23A, 23B, 24F and 35B. In an embodiment the immunogenic composition is a 12-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a Streptococcus pneumoniae serotype 3 glycoconjugate of the invention and further comprising glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 25-valent pneumococcal conjugate compositions.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate from S. pneumoniae serotype 3 is conjugated to CRM₁₉₇. In an embodiment of any of the above immunogenic compositions, the glycoconjugate from S. pneumoniae serotype 3 is conjugated to SCP.

In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM₁₉₇. In an embodiment of any of the above immunogenic compositions, the glycoconjugate from S. pneumoniae serotype 12F is conjugated to CRM₁₉₇. In an embodiment of any of the above immunogenic compositions, the glycoconjugate from S. pneumoniae serotype 10A is conjugated to CRM₁₉₇. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇.

In an embodiment of any of the above immunogenic compositions, the glycoconjugates of any of the above immunogenic compositions are all individually conjugated to CRM₁₉₇.

In an embodiment of any of the above immunogenic compositions, the glycoconjugate from S. pneumoniae serotype 3 is conjugated to SCP and the other glycoconjugate(s) is/are all individually conjugated to CRM₁₉₇.

In an embodiment, the glycoconjugates from S. pneumoniae serotypes 1, 4, 5, 6B, 7F, 9V, 14 and/or 23F of any of the above immunogenic compositions are individually conjugated to PD.

In an embodiment, the glycoconjugate from S. pneumoniae serotype 18C of any of the above immunogenic compositions is conjugated to TT.

In an embodiment, the glycoconjugate from S. pneumoniae serotype 19F of any of the above immunogenic compositions is conjugated to DT.

In an embodiment, the glycoconjugates from S. pneumoniae serotypes 1, 4, 5, 6B, 7F, 9V, 14 and/or 23F of any of the above immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment the above immunogenic compositions comprise from 8 to 20 different serotypes of S. pneumoniae.

In an embodiment of any of the above immunogenic compositions, the glycoconjugate from S. pneumoniae serotype 3 is conjugated to SCP, at least one other glycoconjugate is conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, one other glycoconjugate is conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least two other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, two other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least three other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, three other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least four other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, four other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least five other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, five other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least one other glycoconjugate is conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, one other glycoconjugate is conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least two other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, two other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least three other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, three other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least four other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, four other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least five other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, five other glycoconjugates are conjugated to SCP and the other glycoconjugate(s) is/are all individually conjugated to CRM₁₉₇.

Compositions of the invention may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

2.2 Dosage of the Immunogenic Compositions of the Invention

The amount of glycoconjugate(s) in each dose is selected as an amount which induces an immunoprotective response without significant, adverse side effects in typical vaccines. Such amount will vary depending upon which specific immunogen is employed and how it is presented.

The amount of a particular glycoconjugate in an immunogenic composition can be calculated based on total polysaccharide for that conjugate (conjugated and nonconjugated). For example, a glycoconjugate with 20% free polysaccharide will have about 80 μg of conjugated polysaccharide and about 20 μg of nonconjugated polysaccharide in a 100 μg polysaccharide dose. The amount of glycoconjugate can vary depending upon the pneumococcal serotype. The saccharide concentration can be determined by the uronic acid assay.

The “immunogenic amount” of the different polysaccharide components in the immunogenic composition, may diverge and each may comprise about 0.5 μg, about 0.75 μg, about 1 μg, about 2 μg, about 3 μg, about 4 μg, about 5 μg, about 6 μg, about 7 μg, about 8 μg, about 9 μg, about 10 μg, about 15 μg, about 20 μg, about 30 μg, about 40 μg, about 50 μg, about 60 μg, about 70 μg, about 80 μg, about 90 μg, or about 100 μg of any particular polysaccharide antigen.

Generally, each dose will comprise 0.1 μg to 100 μg of serotype 3 polysaccharide. In an embodiment each dose will comprise 0.1 μg to 100 μg of serotype 3 polysaccharide. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of serotype 3 polysaccharide. Any whole number integer within any of the above ranges is contemplated as an embodiment of the disclosure.

In an embodiment, each dose will comprise about 0.5 μg of serotype 3 polysaccharide.

In an embodiment, each dose will comprise about 0.55 μg of serotype 3 polysaccharide.

In an embodiment, each dose will comprise about 0.75 μg of serotype 3 polysaccharide.

In an embodiment, each dose will comprise about 1.0 μg of serotype 3 polysaccharide.

In an embodiment, each dose will comprise about 1.1 μg of serotype 3 polysaccharide.

In an embodiment, each dose will comprise about 1.5 μg of serotype 3 polysaccharide.

In an embodiment, each dose will comprise about 2.0 μg of serotype 3 polysaccharide.

In an embodiment, each dose will comprise about 2.2 μg of serotype 3 polysaccharide.

In an embodiment, each dose will comprise about 2.5 μg of serotype 3 polysaccharide.

In an embodiment, each dose will comprise about 3.0 μg of serotype 3 polysaccharide.

In an embodiment, each dose will comprise about 3.5 μg of serotype 3 polysaccharide.

In an embodiment, each dose will comprise about 4.0 μg of serotype 3 polysaccharide.

In an embodiment, each dose will comprise about 4.4 μg of serotype 3 polysaccharide.

In an embodiment, each dose will comprise about 5.0 μg of serotype 3 polysaccharide.

In an embodiment, each dose will comprise about 5.5 μg of serotype 3 polysaccharide.

In an embodiment, each dose will comprise about 6.0 μg of serotype 3 polysaccharide.

Generally, each dose will comprise 0.1 μg to 100 μg of polysaccharide for a given serotype. In an embodiment each dose will comprise 0.1 μg to 100 μg of polysaccharide for a given serotype. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of polysaccharide for a given serotype. Any whole number integer within any of the above ranges is contemplated as an embodiment of the disclosure.

In an embodiment, each dose will comprise about 0.5 μg of polysaccharide for each particular glycoconjugate. In an embodiment, each dose will comprise about 0.55 μg of polysaccharide for each particular glycoconjugate. In an embodiment, each dose will comprise about 0.75 μg of polysaccharide for each particular glycoconjugate. In an embodiment, each dose will comprise about 1.0 μg of polysaccharide for each particular glycoconjugate. In an embodiment, each dose will comprise about 1.1 μg of polysaccharide for each particular glycoconjugate. In an embodiment, each dose will comprise about 1.5 μg of polysaccharide for each particular glycoconjugate. In an embodiment, each dose will comprise about 2.0 μg of polysaccharide for each particular glycoconjugate. In an embodiment, each dose will comprise about 2.2 μg of polysaccharide for each particular glycoconjugate. In an embodiment, each dose will comprise about 2.5 μg of polysaccharide for each particular glycoconjugate. In an embodiment, each dose will comprise about 3.0 μg of polysaccharide for each particular glycoconjugate. In an embodiment, each dose will comprise about 3.5 μg of polysaccharide for each particular glycoconjugate. In an embodiment, each dose will comprise about 4.0 μg of polysaccharide for each particular glycoconjugate. In an embodiment, each dose will comprise about 4.4 μg of polysaccharide for each particular glycoconjugate. In an embodiment, each dose will comprise about 5.0 μg of polysaccharide for each particular glycoconjugate. In an embodiment, each dose will comprise about 5.5 μg of polysaccharide for each particular glycoconjugate. In an embodiment, each dose will comprise about 6.0 μg of polysaccharide for each particular glycoconjugate.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotype 1, 3, 4, 5, 6A, 7F, 8, 9V, 10A, 11A, 12F, 14, 15B, 18C, 19A, 19F, 22F, 23F and/or 33F.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotype 1, 4, 5, 6A, 7F, 8, 9V, 10A, 11A, 12F, 14, 15B, 18C, 19A, 19F, 22F, 23F and/or 33F.

In an embodiment, each dose will comprise about 1.0 μg, about 1.1 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.4 μg, about 4.6 μg, about 4.8 μg, about 5.0, about 5.2 μg, about 5.4 μg, about 5.6 μg, about 5.8 μg or about 6.0 μg of polysaccharide for glycoconjugates from S. pneumoniae serotype 6B.

In an embodiment, each dose will comprise about 0.5 μg to about 1.0 μg of polysaccharide for each glycoconjugate from S. pneumoniae serotype 1, 3, 4, 5, 6A, 7F, 8, 9V, 10A, 11A, 12F, 14, 15B, 18C, 19A, 19F, 22F, 23F and 33F, and about 1.0 μg to about 2.0 μg of polysaccharide for glycoconjugate from S. pneumoniae serotype 6B.

In an embodiment, each dose will comprise about 1.5 μg to about 3.0 μg of polysaccharide for each glycoconjugate from S. pneumoniae serotype 1, 3, 4, 5, 6A, 7F, 8, 9V, 10A, 11A, 12F, 14, 15B, 18C, 19A, 19F, 22F, 23F and 33F, and about 3.0 μg to about 6.0 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotype 1, 3, 4, 5, 6A, 7F, 8, 9V, 10A, 11A, 12F, 14, 15B, 18C, 19A, 19F, 22F, 23F and 33F, and about 4.0 μg to about 4.8 μg of polysaccharide for glycoconjugate from S. pneumoniae serotype 6B.

In an embodiment, each dose will comprise about 2.2 μg of polysaccharide from each glycoconjugate from S. pneumoniae serotype 1, 3, 4, 5, 6A, 7F, 8, 9V, 10A, 11A, 12F, 14, 15B, 18C, 19A, 19F, 22F, 23F and 33F, and about 4.4 μg of polysaccharide for glycoconjugate from S. pneumoniae serotype 6B.

In an embodiment, each dose will comprise about 0.5 μg to about 1.0 μg of polysaccharide for each glycoconjugate from S. pneumoniae serotype 1, 3, 4, 5, 6A, 7F, 9V, 14, 15B, 18C, 19A, 19F, 22F, 23F and 33F, and about 1.0 μg to about 2.0 μg of polysaccharide for glycoconjugate from S. pneumoniae serotype 6B.

In an embodiment, each dose will comprise about 1.5 μg to about 3.0 μg of polysaccharide for each glycoconjugate from S. pneumoniae serotype 1, 3, 4, 5, 6A, 7F, 9V, 14, 15B, 18C, 19A, 19F, 22F, 23F and 33F, and about 3 μg to about 6 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotype 1, 3, 4, 5, 6A, 7F, 9V, 14, 15B, 18C, 19A, 19F, 22F, 23F and 33F, and about 4.0 μg to about 4.8 μg of polysaccharide for glycoconjugate from S. pneumoniae serotype 6B.

In an embodiment, each dose will comprise about 2.0 μg of polysaccharide from each glycoconjugate from S. pneumoniae serotype 1, 3, 4, 5, 6A, 7F, 9V, 14, 18C, 19A, 19F, 22F, 23F and 33F, and about 4.0 μg of polysaccharide for glycoconjugate from S. pneumoniae serotype 6B.

In an embodiment, each dose will comprise about 2.2 μg of polysaccharide from each glycoconjugate from S. pneumoniae serotype 1, 3, 4, 5, 6A, 7F, 9V, 14, 15B, 18C, 19A, 19F, 22F, 23F and 33F, and about 4.4 μg of polysaccharide for glycoconjugate from S. pneumoniae serotype 6B.

In an embodiment, each dose will comprise about 0.5 μg to about 1.0 μg of polysaccharide for each glycoconjugate from S. pneumoniae serotype 1, 4, 5, 6A, 7F, 8, 9V, 10A, 11A, 12F, 14, 15B, 18C, 19A, 19F, 22F, 23F and 33F, and about 1.0 μg to about 2.0 μg of polysaccharide for glycoconjugate from S. pneumoniae serotype 6B.

In an embodiment, each dose will comprise about 1.5 μg to about 3.0 μg of polysaccharide for each glycoconjugate from S. pneumoniae serotype 1, 4, 5, 6A, 7F, 8, 9V, 10A, 11A, 12F, 14, 15B, 18C, 19A, 19F, 22F, 23F and 33F, and about 3.0 μg to about 6.0 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotype 1, 4, 5, 6A, 7F, 8, 9V, 10A, 11A, 12F, 14, 15B, 18C, 19A, 19F, 22F, 23F and 33F, and about 4.0 μg to about 4.8 μg of polysaccharide for glycoconjugate from S. pneumoniae serotype 6B.

In an embodiment, each dose will comprise about 2.2 μg of polysaccharide from each glycoconjugate from S. pneumoniae serotype 1, 4, 5, 6A, 7F, 8, 9V, 10A, 11A, 12F, 14, 15B, 18C, 19A, 19F, 22F, 23F and 33F, and about 4.4 μg of polysaccharide for glycoconjugate from S. pneumoniae serotype 6B.

In an embodiment, each dose will comprise about 0.5 μg to about 1.0 μg of polysaccharide for each glycoconjugate from S. pneumoniae serotype 1, 4, 5, 6A, 7F, 9V, 14, 15B, 18C, 19A, 19F, 22F, 23F and 33F, and about 1.0 μg to about 2.0 μg of polysaccharide for glycoconjugate from S. pneumoniae serotype 6B.

In an embodiment, each dose will comprise about 1.5 μg to about 3.0 μg of polysaccharide for each glycoconjugate from S. pneumoniae serotype 1, 4, 5, 6A, 7F, 9V, 14, 15B, 18C, 19A, 19F, 22F, 23F and 33F, and about 3.0 μg to about 6.0 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotype 1, 4, 5, 6A, 7F, 9V, 14, 15B, 18C, 19A, 19F, 22F, 23F and 33F, and about 4.0 μg to about 4.8 μg of polysaccharide for glycoconjugate from S. pneumoniae serotype 6B.

In an embodiment, each dose will comprise about 2.2 μg of polysaccharide from each glycoconjugate from S. pneumoniae serotype 1, 4, 5, 6A, 7F, 9V, 14, 15B, 18C, 19A, 19F, 22F, 23F and 33F, and about 4.4 μg of polysaccharide for glycoconjugate from S. pneumoniae serotype 6B.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and/or 35B.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and/or 35B.

In an embodiment, each dose will comprise about 1.0 μg, about 1.1 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.4 μg, about 4.6 μg, about 4.8 μg, about 5.0, about 5.2 μg, about 5.4 μg, about 5.6 μg, about 5.8 μg or about 6.0 μg of polysaccharide for glycoconjugates from S. pneumoniae serotype 6B.

In an embodiment, each dose will comprise about 1.5 μg to about 3.0 μg of polysaccharide for each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 3.0 μg to about 6.0 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg to about 4.8 μg of polysaccharide for glycoconjugate from S. pneumoniae serotype 6B.

In an embodiment, each dose will comprise about 2.2 μg of polysaccharide from each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.4 μg of polysaccharide for glycoconjugate from S. pneumoniae serotype 6B.

In an embodiment, each dose will comprise about 0.5 μg to about 1.0 μg of polysaccharide for each glycoconjugate from S. pneumoniae serotypes 1, 4, 5, 6A, 7F, 8, 9V, 10A, 11A, 12F, 14, 15B, 18C, 19A, 19F, 22F, 23F and 33F, and about 1.0 μg to about 2.0 μg of polysaccharide for glycoconjugate from S. pneumoniae serotype 6B.

In an embodiment, each dose will comprise about 1.5 μg to about 3.0 μg of polysaccharide for each glycoconjugate from S. pneumoniae serotypes 1, 4, 5, 6A, 7F, 8, 9V, 10A, 11A, 12F, 14, 15B, 18C, 19A, 19F, 22F, 23F and 33F, and about 3.0 μg to about 6.0 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotypes 1, 4, 5, 6A, 7F, 8, 9V, 10A, 11A, 12F, 14, 15B, 18C, 19A, 19F, 22F, 23F and 33F, and about 4.0 μg to about 4.8 μg of polysaccharide for glycoconjugate from S. pneumoniae serotype 6B.

In an embodiment, each dose will comprise about 2.2 μg of polysaccharide from each glycoconjugate from S. pneumoniae serotypes 1, 4, 5, 6A, 7F, 8, 9V, 10A, 11A, 12F, 14, 15B, 18C, 19A, 19F, 22F, 23F and 33F, and about 4.4 μg of polysaccharide for glycoconjugate from S. pneumoniae serotype 6B.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and/or 38.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotypes 2, 3, 7C, 9N, 10B, 15A, 16F, 17F, 20, 21, 22A, 23A, 23B, 24B, 24F, 27, 29, 31, 33B, 34, 35B, 35F and/or 38.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotypes 2, 3, 7C, 9N, 10B, 15A, 16F, 17F, 19A, 19F, 20, 21, 22A, 23A, 23B, 24B, 24F, 27, 29, 31, 33B, 34, 35B, 35F and/or 38.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotypes 2, 3, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 0.5 μg, about 0.55 μg, about 0.75 μg, about 1.1 μg, about 1.2 μg, about 1.3 μg, about 1.4 μg, about 1.5 μg, about 1.6 μg, about 1.7 μg, about 1.8 μg, about 1.9 μg, about 2.0 μg, about 2.1 μg, about 2.2 μg, about 2.3 μg, about 2.4 μg, about 2.5 μg, about 2.6 μg, about 2.7 μg, about 2.8 μg, about 2.9 μg, or about 3.0 μg of polysaccharide for glycoconjugates from S. pneumoniae serotypes 2, 3, 7C, 9N, 10B, 15A, 16F, 17F, 19A, 19F, 20, 21, 22A, 23A, 23B, 24B, 24F, 27, 29, 31, 33B, 34, 35B, 35F and 38.

In an embodiment, each dose will comprise about 0.5 μg to about 1.0 μg of polysaccharide for each glycoconjugate from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 1.5 μg to about 3.0 μg of polysaccharide for each glycoconjugate from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 2.0 μg to about 2.5 μg of polysaccharide for each glycoconjugate from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 2.0 μg of polysaccharide from each glycoconjugate from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 2.2 μg of polysaccharide from each glycoconjugate from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 0.5 μg to about 1.0 μg of polysaccharide for each glycoconjugate from S. pneumoniae serotypes 2, 3, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 1.5 μg to about 3.0 μg of polysaccharide for each glycoconjugate from S. pneumoniae serotypes 2, 3, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 2.0 μg to about 2.5 μg of polysaccharide for each glycoconjugate from S. pneumoniae serotypes 2, 3, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 2.0 μg of polysaccharide from each glycoconjugate from S. pneumoniae serotypes 2, 3, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 2.2 μg of polysaccharide from each glycoconjugate from S. pneumoniae serotypes 2, 3, 7C, 9N, 10B, 15A, 16F, 17F, 20, 21, 22A, 23A, 23B, 24B, 24F, 27, 29, 31, 33B, 34, 35B, 35F and 38.

In an embodiment, each dose will comprise about 0.5 μg to about 1.0 μg of polysaccharide for each glycoconjugate from S. pneumoniae serotypes 2, 3, 7C, 9N, 10B, 15A, 16F, 17F, 19A, 19F, 20, 21, 22A, 23A, 23B, 24B, 24F, 27, 29, 31, 33B, 34, 35B, 35F and 38.

In an embodiment, each dose will comprise about 1.5 μg to about 3.0 μg of polysaccharide for each glycoconjugate from S. pneumoniae serotypes 2, 3, 7C, 9N, 10B, 15A, 16F, 17F, 19A, 19F, 20, 21, 22A, 23A, 23B, 24B, 24F, 27, 29, 31, 33B, 34, 35B, 35F and 38.

In an embodiment, each dose will comprise about 2.0 μg to about 2.5 μg of polysaccharide for each glycoconjugate from S. pneumoniae serotypes 2, 3, 7C, 9N, 10B, 15A, 16F, 17F, 19A, 19F, 20, 21, 22A, 23A, 23B, 24B, 24F, 27, 29, 31, 33B, 34, 35B, 35F and 38.

In an embodiment, each dose will comprise about 2.0 μg of polysaccharide from each glycoconjugate from S. pneumoniae serotypes 2, 3, 7C, 9N, 10B, 15A, 16F, 17F, 19A, 19F, 20, 21, 22A, 23A, 23B, 24B, 24F, 27, 29, 31, 33B, 34, 35B, 35F and 38.

In an embodiment, each dose will comprise about 2.2 μg of polysaccharide from each glycoconjugate from S. pneumoniae serotypes 2, 3, 7C, 9N, 10B, 15A, 16F, 17F, 19A, 19F, 20, 21, 22A, 23A, 23B, 24B, 24F, 27, 29, 31, 33B, 34, 35B, 35F and 38.

2.3 Carrier Amount

Generally, each dose will comprise 10 μg to 150 μg of carrier protein, particularly 15 μg to 100 μg of carrier protein, more particularly 25 μg to 75 μg of carrier protein, and even more particularly 40 μg to 60 μg of carrier protein. In an embodiment, said carrier protein is CRM₁₉₇. In an embodiment, said carrier protein is SCP.

In an embodiment, each dose will comprise about 10 μg, about 15 μg, about 20 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg or about 75 μg of carrier protein.

In an embodiment, each dose will comprise about 30 μg of carrier protein. In an embodiment, each dose will comprise about 31 μg of carrier protein. In an embodiment, each dose will comprise about 32 μg of carrier protein. In an embodiment, each dose will comprise about 33 μg of carrier protein. In an embodiment, each dose will comprise about 34 μg of carrier protein. In an embodiment, each dose will comprise about 45 μg of carrier protein.

In an embodiment, each dose will comprise about 40 μg of carrier protein. In an embodiment, each dose will comprise about 41 μg of carrier protein. In an embodiment, each dose will comprise about 42 μg of carrier protein. In an embodiment, each dose will comprise about 43 μg of carrier protein. In an embodiment, each dose will comprise about 44 μg of carrier protein. In an embodiment, each dose will comprise about 45 μg of carrier protein.

In an embodiment, each dose will comprise about 48 μg of carrier protein. In an embodiment, each dose will comprise about 49 μg of carrier protein. In an embodiment, each dose will comprise about 50 μg of carrier protein. In an embodiment, each dose will comprise about 51 μg of carrier protein. In an embodiment, each dose will comprise about 52 μg of carrier protein. In an embodiment, each dose will comprise about 53 μg of carrier protein.

In an embodiment, said carrier protein is CRM₁₉₇.

In an embodiment, said carrier protein is SCP.

2.4 Further Antigens

Immunogenic compositions of the invention comprise conjugated S. pneumoniae saccharide antigen(s) (glycoconjugate(s)). They may also further include antigen(s) from other pathogen(s), particularly from bacteria and/or viruses. Preferred further antigens are selected from: a diphtheria toxoid (D), a tetanus toxoid (T), a pertussis antigen (P), which is typically acellular (Pa), a hepatitis B virus (HBV) surface antigen (HBsAg), a hepatitis A virus (HAV) antigen, a conjugated Haemophilus influenzae type b capsular saccharide (Hib), inactivated poliovirus vaccine (IPV).

In an embodiment, the immunogenic compositions of the invention comprise D-T-Pa. In an embodiment, the immunogenic compositions of the invention comprise D-T-Pa-Hib, D-T-Pa-IPV or D-T-Pa-HBsAg. In an embodiment, the immunogenic compositions of the invention comprise D-T-Pa-HBsAg-IPV or D-T-Pa-HBsAg-Hib. In an embodiment, the immunogenic compositions of the invention comprise D-T-Pa-HBsAg-IPV-Hib.

Pertussis antigens: Bordetella pertussis causes whooping cough. Pertussis antigens in vaccines are either cellular (whole cell, in the form of inactivated B. pertussis cells) or acellular. Preparation of cellular pertussis antigens is well documented (e.g., it may be obtained by heat inactivation of phase I culture of B. pertussis). Preferably, however, the invention uses acellular antigens. Where acellular antigens are used, it is preferred to use one, two or (preferably) three of the following antigens: (1) detoxified pertussis toxin (pertussis toxoid, or PT); (2) filamentous hemagglutinin (FHA); (3) pertactin (also known as the 69 kilodalton outer membrane protein). FHA and pertactin may be treated with formaldehyde prior to use according to the invention. PT is preferably detoxified by treatment with formaldehyde and/or glutaraldehyde. Acellular pertussis antigens are preferably adsorbed onto one or more aluminum salt adjuvants. As an alternative, they may be added in an unadsorbed state. Where pertactin is added then it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is most preferred.

Inactivated poliovirus vaccine: Poliovirus causes poliomyelitis. Rather than use oral poliovirus vaccine, preferred embodiments of the invention use IPV. Prior to administration to patients, polioviruses must be inactivated, and this can be achieved by treatment with formaldehyde. Poliomyelitis can be caused by one of three types of poliovirus. The three types are similar and cause identical symptoms, but they are antigenically different and infection by one type does not protect against infection by others. It is therefore preferred to use three poliovirus antigens in the invention: poliovirus Type 1 (e.g., Mahoney strain), poliovirus Type 2 (e.g., MEF-1 strain), and poliovirus Type 3 (e.g., Saukett strain). The viruses are preferably grown, purified and inactivated individually, and are then combined to give a bulk trivalent mixture for use with the invention.

Diphtheria toxoid: Corynebacterium diphtheriae causes diphtheria. Diphtheria toxin can be treated (e.g., using formalin or formaldehyde) to remove toxicity while retaining the ability to induce specific anti-toxin antibodies after injection. These diphtheria toxoids are used in diphtheria vaccines. Preferred diphtheria toxoids are those prepared by formaldehyde treatment. The diphtheria toxoid can be obtained by growing C. diphtheriae in growth medium, followed by formaldehyde treatment, ultrafiltration and precipitation. The toxoided material may then be treated by a process comprising sterile filtration and/or dialysis. The diphtheria toxoid is preferably adsorbed onto an aluminum hydroxide adjuvant.

Tetanus toxoid: Clostridium tetani causes tetanus. Tetanus toxin can be treated to give a protective toxoid. The toxoids are used in tetanus vaccines. Preferred tetanus toxoids are those prepared by formaldehyde treatment. The tetanus toxoid can be obtained by growing C. tetani in growth medium, followed by formaldehyde treatment, ultrafiltration and precipitation. The material may then be treated by a process comprising sterile filtration and/or dialysis.

Hepatitis A virus antigens: Hepatitis A virus (HAV) is one of the known agents which causes viral hepatitis. A preferred HAV component is based on inactivated virus, and inactivation can be achieved by formalin treatment.

Hepatitis B virus (HBV) is one of the known agents which causes viral hepatitis. The major component of the capsid is a protein known as HBV surface antigen or, more commonly, HBsAg, which is typically a 226-amino acid polypeptide with a molecular weight of ˜24 kDa. All existing hepatitis B vaccines contain HBsAg, and when this antigen is administered to a normal vaccine it stimulates the production of anti-HBsAg antibodies which protect against HBV infection.

For vaccine manufacture, HBsAg has been made in two ways: purification of the antigen in particulate form from the plasma of chronic hepatitis B carriers or expression of the protein by recombinant DNA methods (e.g., recombinant expression in yeast cells). Unlike native HBsAg (i.e., as in the plasma-purified product), yeast-expressed HBsAg is generally non-glycosylated, and this is the most preferred form of HBsAg for use with the invention.

Conjugated Haemophilus influenzae type b antigens: Haemophilus influenzae type b (Hib) causes bacterial meningitis. Hib vaccines are typically based on the capsular saccharide antigen, the preparation of which is well documented. The Hib saccharide can be conjugated to a carrier protein in order to enhance its immunogenicity, especially in children. Typical carrier proteins are tetanus toxoid, diphtheria toxoid, CRM₁₉₇, H. influenzae protein D, and an outer membrane protein complex from serogroup B meningococcus. The saccharide moiety of the conjugate may comprise full-length polyribosylribitol phosphate (PRP) as prepared from Hib bacteria, and/or fragments of full-length PRP. Hib conjugates may or may not be adsorbed to an aluminum salt adjuvant.

In an embodiment the immunogenic compositions of the invention further include a conjugated N. meningitidis serogroup Y capsular saccharide (MenY), and/or a conjugated N. meningitidis serogroup C capsular saccharide (MenC).

In an embodiment the immunogenic compositions of the invention further include a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the immunogenic compositions of the invention further include a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

2.5 Adjuvant(s)

In some embodiments, the immunogenic compositions disclosed herein may further comprise at least one, two or three adjuvants. In some embodiments, the immunogenic compositions disclosed herein may further comprise at least one adjuvant. In some embodiments, the immunogenic compositions disclosed herein may further comprise one adjuvant. In some embodiments, the immunogenic compositions disclosed herein may further comprise two adjuvants. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant. In a preferred embodiment, the immunogenic compositions disclosed herein comprise aluminum phosphate as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, Mass.), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068. In an embodiment of the present invention, the immunogenic compositions as disclosed herein comprise any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

In an embodiment of the present invention, the immunogenic compositions as disclosed herein comprise an A class CpG oligonucleotide. Preferably, the “A class” CpG oligonucleotide of the invention has the following nucleic acid sequence: 5′ GGGGACGACGTCGTGGGGGGG 3′ (SEQ ID NO: 1). Some non-limiting examples of A-Class oligonucleotides include: 5′ G*G*G_G_A_C_G_A_C_G_T_C_G_T_G_G*G*G*G*G*G 3′ (SEQ ID NO: 2); wherein “*” refers to a phosphorothioate bond and “_” refers to a phosphodiester bond.

In an embodiment of the present invention, the immunogenic compositions as disclosed herein comprise a B class CpG Oligonucleotide. In one embodiment, the CpG oligonucleotide for use in the present invention is a B class CpG oligonucleotide represented by at least the formula:

5′ X₁X₂CGX₃X₄ 3′, wherein X₁, X₂, X₃, and X₄ are nucleotides. In one embodiment, X₂ is adenine, guanine, or thymine. In another embodiment, X₃ is cytosine, adenine, or thymine.

The B class CpG oligonucleotide sequences of the invention are those broadly described above as well as disclosed in WO 96/02555, WO 98/18810 and U.S. Pat. Nos. 6,194,388; 6,207,646; 6,214,806; 6,218,371; 6,239,116 and 6,339,068. Exemplary sequences include but are not limited to those disclosed in these latter applications and patents.

In an embodiment, the “B class” CpG oligonucleotide of the invention has the following nucleic acid sequence:

(SEQ ID NO: 3)
5′ TCGTCGTTTTTCGGTGCTTTT 3′,
or
(SEQ ID NO: 4)
5′ TCGTCGTTTTTCGGTCGTTTT 3′,
or
(SEQ ID NO: 5)
5′ TCGTCGTTTTGTCGTTTTGTCGTT 3′,
or
(SEQ ID NO: 6)
5′ TCGTCGTTTCGTCGTTTTGTCGTT 3′,
or
(SEQ ID NO: 7)
5′ TCGTCGTTTTGTCGTTTTTTTCGA 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

Some non-limiting examples of B-Class oligonucleotides include:

(SEQ ID NO: 8)
5′ T*C*G*T*C*G*T*T*T*T*T*C*G
*G*T*G*C*T*T*T*T 3′,
or
(SEQ ID NO: 9)
5′ T*C*G*T*C*G*T*T*T*T*T*C*
G*G*T*C*G*T*T*T*T 3′,
or
(SEQ ID NO: 10)
5′ T*C*G*T*C*G*T*T*T*T*G*T*
C*G*T*T*T*T*G*T*C*G*T*T 3′,
or
(SEQ ID NO: 11)
5′ T*C*G*T*C*G*T*T*T*C*G*T*
C*G*T*T*T*T*G*T*C*G*T*T 3′,
or
(SEQ ID NO: 12)
5′ T*C*G*T*C*G*T*T*T*T*G*T*
C*G*T*T*T*T*T*T*T*C*G*A 3′.

wherein “*” refers to a phosphorothioate bond.

In an embodiment of the present invention, the immunogenic compositions as disclosed herein comprise a C class CpG Oligonucleotide. In an embodiment, the “C class” CpG oligonucleotides of the invention have the following nucleic acid sequence:

(SEQ ID NO: 13)
5′ TCGCGTCGTTCGGCGCGCGCCG 3′,
or
(SEQ ID NO: 14)
5′ TCGTCGACGTTCGGCGCGCGCCG 3′,
or
(SEQ ID NO: 15)
5′ TCGGACGTTCGGCGCGCGCCG 3′ ,
or
(SEQ ID NO: 16)
5′ TCGGACGTTCGGCGCGCCG 3′,
or
(SEQ ID NO: 17)
5′ TCGCGTCGTTCGGCGCGCCG 3′,
or
(SEQ ID NO: 18)
5′ TCGACGTTCGGCGCGCGCCG 3′,
or
(SEQ ID NO: 19)
5′ TCGACGTTCGGCGCGCCG 3′,
or
(SEQ ID NO: 20)
5′ TCGCGTCGTTCGGCGCCG 3′,
or
(SEQ ID NO: 21)
5′ TCGCGACGTTCGGCGCGCGCCG 3′,
or
(SEQ ID NO: 22)
5′ TCGTCGTTTTCGGCGCGCGCCG 3′,
or
(SEQ ID NO: 23)
5′ TCGTCGTTTTCGGCGGCCGCCG 3′,
or
(SEQ ID NO: 24)
5′ TCGTCGTTTTACGGCGCCGTGCCG 3′,
or
(SEQ ID NO: 25)
5′ TCGTCGTTTTCGGCGCGCGCCGT 3′

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide.

Some non-limiting examples of C-Class oligonucleotides include:

(SEQ ID NO: 26)
5′ T*C_G*C_G*T*C_G*T*T*C_G*
G*C*G*C_G*C*G*C*C*G 3′,
or
(SEQ ID NO: 27)
5′ T*C_G*T*C_G*A*C_G*T*T*C_
G*G*C*G*C_G*C*G*C*C*G 3′,
or
(SEQ ID NO: 28)
5′ T*C_G*G*A*C_G*T*T*C_G*G*
C*G*C_G*C*G*C*C*G 3′,
or
(SEQ ID NO: 29)
5′ T*C_G*G*A*C_G*T*T*C_G*G*
C*G*C*G*C*C*G 3′,
or
(SEQ ID NO: 30)
5′ T*C_G*C_G*T*C_G*T*T*C_G*
G*C*G*C*G*C*C*G 3′,
or
(SEQ ID NO: 31)
5′ T*C_G*A*C_G*T*T*C_G*G*C*
G*C_G*C*G*C*C*G 3′,
or
(SEQ ID NO: 32)
5′ T*C_G*A*C_G*T*T*C_G*G*C*
G*C*G*C*C*G 3′,
or
(SEQ ID NO: 33)
5′ T*C_G*C_G*T*C_G*T*T*C_G*
G*C*G*C*C*G 3′,
or
(SEQ ID NO: 34)
5′ T*C_G*C_G*A*C_G*T*T*C_G*G*
C*G*C_G*C*G*C*C*G 3′,
or
(SEQ ID NO: 35)
5′ T*C*G*T*C*G*T*T*T*T*C*G*G*
C*G*C*G*C*G*C*C*G 3′,
or
(SEQ ID NO: 36)
5′ T*C*G*T*C*G*T*T*T*T*C*G*G*
C*G*G*C*C*G*C*C*G 3′,
or
(SEQ ID NO: 37)
5′ T*C*G*T*C_G*T*T*T*T*A*C_G*
G*C*G*C*C_G*T*G*C*C*G 3′,
or
(SEQ ID NO: 38)
5′ T*C_G*T*C*G*T*T*T*T*C*G*G*
C*G*C*G*C*G*C*C*G*T 3′

wherein “*” refers to a phosphorothioate bond and “_” refers to a phosphodiester bond. In any of these sequences, an ethyl-uridine or a halogen may substitute for the 5′ T;

examples of halogen substitutions include but are not limited to bromo-uridine or iodo-uridine substitutions.

In an embodiment of the present invention, the immunogenic compositions as disclosed herein comprise a P class CpG Oligonucleotide. In an embodiment, the CpG oligonucleotide for use in the present invention is a P class CpG oligonucleotide containing a 5′ TLR activation domain and at least two palindromic regions, one palindromic region being a 5′ palindromic region of at least 6 nucleotides in length and connected to a 3′ palindromic region of at least 8 nucleotides in length either directly or through a spacer, wherein the oligonucleotide includes at least one YpR dinucleotide. In an embodiment, said oligonucleotide is not T*C_G*T*C_G*A*C_G*T*T*C_G*G*C*G*C_G*C*G*C*C*G (SEQ ID NO: 27). In one embodiment the P class CpG oligonucleotide includes at least one unmethylated CpG dinucleotide. In another embodiment the TLR activation domain is TCG, TTCG, TTTCG, TYpR, TTYpR, TTTYpR, UCG, UUCG, UUUCG, TTT, or TTTT. In yet another embodiment the TLR activation domain is within the 5′ palindromic region. In another embodiment the TLR activation domain is immediately 5′ to the 5′ palindromic region. In an embodiment, the “P class” CpG oligonucleotides of the invention have the following nucleic acid sequence: 5′ TCGTCGACGATCGGCGCGCGCCG 3′ (SEQ ID NO: 39).

In said sequences, all of the linkages may be all phosphorothioate bonds. In another embodiment, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

A non-limiting example of P-Class oligonucleotides include:

(SEQ ID NO: 40)
5′ T*C_G*T*C_G*A*C_G*A*T*C_G*
G*C*G*C_G*C*G*C*C*G 3′

wherein “*” refers to a phosphorothioate bond and “_” refers to a phosphodiester bond. In one embodiment the oligonucleotide includes at least one phosphorothioate linkage. In another embodiment all internucleotide linkages of the oligonucleotide are phosphorothioate linkages. In another embodiment the oligonucleotide includes at least one phosphodiester-like linkage. In another embodiment the phosphodiester-like linkage is a phosphodiester linkage. In another embodiment a lipophilic group is conjugated to the oligonucleotide. In one embodiment the lipophilic group is cholesterol.

In an embodiment, all the internucleotide linkages of the CpG oligonucleotides disclosed herein are phosphodiester bonds (“soft” oligonucleotides, as described in WO 2007/026190). In another embodiment, CpG oligonucleotides of the invention are rendered resistant to degradation (e.g., are stabilized). A “stabilized oligonucleotide” refers to an oligonucleotide that is relatively resistant to in vivo degradation (e.g., via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases.

The immunostimulatory oligonucleotides may have a chimeric backbone, which have combinations of phosphodiester and phosphorothioate linkages. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. When the phosphodiester linkage is preferentially located within the CpG motif such molecules are called “semi-soft” as described in WO 2007/026190.

Other modified oligonucleotides include combinations of phosphodiester, phosphorothioate, methylphosphonate, methylphosphorothioate, phosphorodithioate, and/or p-ethoxy linkages.

Mixed backbone modified ODN may be synthesized as described in WO 2007/026190. The size of the CpG oligonucleotide (i.e., the number of nucleotide residues along the length of the oligonucleotide) also may contribute to the stimulatory activity of the oligonucleotide. For facilitating uptake into cells, CpG oligonucleotide of the invention preferably have a minimum length of 6 nucleotide residues. Oligonucleotides of any size greater than 6 nucleotides (even many kb long) are capable of inducing an immune response if sufficient immunostimulatory motifs are present, because larger oligonucleotides are degraded inside cells. In certain embodiments, the CpG oligonucleotides are 6 to 100 nucleotides long, preferentially 8 to 30 nucleotides long. In important embodiments, nucleic acids and oligonucleotides of the invention are not plasmids or expression vectors.

In an embodiment, the CpG oligonucleotide disclosed herein comprise substitutions or modifications, such as in the bases and/or sugars as described at paragraphs 134 to 147 of WO 2007/026190.

In an embodiment, the CpG oligonucleotide of the present invention is chemically modified. Examples of chemical modifications are known to the skilled person and are described, for example in Uhlmann et al. (1990) Chem. Rev. 90:543; S. Agrawal, Ed., Humana Press, Totowa, USA 1993; Crooke et al. (1996) Annu. Rev. Pharmacol. Toxicol. 36:107-129; and Hunziker et al. (1995) Mod. Synth. Methods 7:331-417. An oligonucleotide according to the invention may have one or more modifications, wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

In some embodiments of the invention, CpG-containing nucleic acids might be simply mixed with immunogenic carriers according to methods known to those skilled in the art (see, e.g., WO 03/024480).

In a particular embodiment of the present invention, any of the immunogenic compositions disclosed herein comprise from 2 μg to 100 mg of CpG oligonucleotide. In a particular embodiment of the present invention, the immunogenic composition of the invention comprises 0.1 mg to 50 mg of CpG oligonucleotide, preferably from 0.2 mg to 10 mg CpG oligonucleotide, more preferably from 0.3 mg to 5 mg CpG oligonucleotide. In a particular embodiment of the present invention, the immunogenic composition of the invention comprises from 0.3 mg to 5 mg CpG oligonucleotide. Even preferably, the immunogenic composition of the invention may comprise from 0.5 to 2 mg CpG oligonucleotide. Most preferably, the immunogenic composition of the invention may comprise from 0.75 to 1.5 mg CpG oligonucleotide. In a preferred embodiment, any of the immunogenic composition disclosed herein may comprise about 1 mg CpG oligonucleotide.

3 Formulation

The immunogenic compositions of the invention may be formulated in liquid form (i.e., solutions or suspensions) or in a lyophilized form. In an embodiment, the immunogenic composition of the invention is formulated in a liquid form. In an embodiment, the immunogenic composition of the invention is formulated in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In some embodiments, the buffer is succinate. In some embodiments, the buffer is histidine. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™ 60), polysorbate 65 (TWEEN™ 65), polysorbate 80 (TWEEN™ 80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w). In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0. In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL. In an embodiment, the immunogenic composition of the invention for injection has a volume of 0.2 mL to 1 mL, even more preferably a volume of about 0.5 mL.

4 Uses of the Glycoconjugate and Immunogenic Compositions of the Invention

The glycoconjugates disclosed herein may be use as antigens. For example, they may be part of a vaccine.

Therefore, in an embodiment, the immunogenic compositions of the invention are for use as a medicament.

In an embodiment, the immunogenic compositions of the invention are for use as a vaccine.

Therefore, in an embodiment, the immunogenic compositions described herein are for use in generating an immune response in a subject. In one aspect, the subject is a mammal, such as a human, cat, sheep, pig, horse, bovine or dog. In one aspect, the subject is a human.

The immunogenic compositions described herein may be used in therapeutic or prophylactic methods for preventing, treating or ameliorating a bacterial infection, disease or condition in a subject. In particular, immunogenic compositions described herein may be used to prevent, treat or ameliorate a S. pneumoniae serotype 3 infection, disease or condition in a subject.

Thus, in one aspect, the disclosure provides a method of preventing, treating or ameliorating an infection, disease or condition associated with S. pneumoniae serotype 3 in a subject, comprising administering to the subject an immunologically effective amount of an immunogenic composition of the disclosure.

In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In an embodiment, the disclosure provides a method of inducing an immune response to S. pneumoniae serotype 3 in a subject comprising administering to the subject an immunologically effective amount of an immunogenic composition of the invention. In one aspect, the subject is a mammal, such as a human, cat, sheep, pig, horse, bovine or dog. In one aspect, the subject is a human.

In an embodiment, the immunogenic compositions disclosed herein are for use as a vaccine. In such embodiments the immunogenic compositions described herein may be used to prevent S. pneumoniae serotype 3 infection in a subject. Thus, in one aspect, the invention provides a method of preventing an infection by S. pneumoniae serotype 3 in a subject comprising administering to the subject an immunologically effective amount of an immunogenic composition of the disclosure. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject is a mammal, such as a human, cat, sheep, pig, horse, bovine or dog. In one aspect, the subject is a human.

The immunogenic composition of the present disclosure can be used to protect or treat a human susceptible to a S. pneumoniae serotype 3 infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

5 Subject to be Treated with the Immunogenic Compositions of the Invention

As disclosed herein, the immunogenic compositions described herein may be used in various therapeutic or prophylactic methods for preventing, treating or ameliorating a bacterial infection, disease or condition in a subject.

In a preferred embodiment, said subject is a human. In a most preferred embodiment, said subject is a newborn (i.e., under three months of age), an infant (i.e., from 3 months to one year of age) or a toddler (i.e., from one year to four years of age).

In an embodiment, the immunogenic compositions disclosed herein are for use as a vaccine.

In such embodiment, the subject to be vaccinated may be less than 1 year of age. For example, the subject to be vaccinated can be about 1, about 2, about 3, about 4, about 5, about 6, about 7, about 8, about 9, about 10, about 11 or about 12 months of age. In an embodiment, the subject to be vaccinated is about 2, about 4 or about 6 months of age. In another embodiment, the subject to be vaccinated is less than 2 years of age. For example, the subject to be vaccinated can be about 12 to about 15 months of age. In some cases, as little as one dose of the immunogenic composition according to the invention is needed, but under some circumstances, a second, third or fourth dose may be given (see section 8 below).

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

In an embodiment of the present invention, the immunocompromised subject to be vaccinated suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is selected from the one disclosed on page 24, line 11, to page 25, line 19, of WO 2010/125480.

In a particular embodiment of the present invention, the immunocompromised subject to be vaccinated suffers from a disease selected from the groups consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised subject to be vaccinated suffers from malnutrition.

In a particular embodiment of the present invention, the immunocompromised subject to be vaccinated is taking a drug or treatment that lowers the body's resistance to infection. In an embodiment, said drug is selected from the one disclosed on page 26, line 33, to page 26, line 4, of WO 2010/125480.

In a particular embodiment of the present invention, the immunocompromised subject to be vaccinated is a smoker.

In a particular embodiment of the present invention, the immunocompromised subject to be vaccinated has a white blood cell count (leukocyte count) below 5×10⁹ cells per liter, or below 4×10⁹ cells per liter, or below 3×10⁹ cells per liter, or below 2×10⁹ cells per liter, or below 1×10⁹ cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised subject to be vaccinated suffers from neutropenia. In a particular embodiment of the present invention, the immunocompromised subject to be vaccinated has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter.

A low white blood cell count or “neutropenia” is a condition characterized by abnormally low levels of neutrophils in the circulating blood. Neutrophils are a specific kind of white blood cell that help to prevent and fight infections. The most common reason that cancer patients experience neutropenia is as a side effect of chemotherapy. Chemotherapy-induced neutropenia increases a patient's risk of infection and disrupts cancer treatment. In a particular embodiment of the present invention, the immunocompromised subject to be vaccinated has a CD4+ cell count below 500/mm³, or CD4+ cell count below 300/mm³, or CD4+ cell count below 200/mm³, CD4+ cell count below 100/mm³, CD4+ cell count below 75/mm³, or CD4+ cell count below 50/mm³.

CD4 cell tests are normally reported as the number of cells in mm³. Normal CD4 counts are between 500 and 1,600, and CD8 counts are between 375 and 1,100. CD4 counts drop dramatically in people with HIV.

In an embodiment of the invention, any of the immunocompromised subjects disclosed herein is a human male or a human female.

6 Regimen

In some cases, as little as one dose of the immunogenic composition according to the invention is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the invention is a single dose. In a particular embodiment, said single dose schedule is for healthy persons being at least 2 years of age.

In an embodiment, the schedule of vaccination of the immunogenic composition according to the invention is a multiple dose schedule. In a particular embodiment, said multiple dose schedule consists of a series of 2 doses separated by an interval of about 1 month to about 2 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses separated by an interval of about 1 month to about 2 months. In another embodiment, said multiple dose schedule consists of a series of 3 doses separated by an interval of about 1 month, or a series of 3 doses separated by an interval of about 2 months.

In another embodiment, said multiple dose schedule consists of a series of 3 doses separated by an interval of about 1 month to about 2 months followed by a fourth dose about 10 months to about 13 months after the first dose. In another embodiment, said multiple dose schedule consists of a series of 3 doses separated by an interval of about 1 month followed by a fourth dose about 10 months to about 13 months after the first dose, or a series of 3 doses separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses separated by an interval of about 1 month to about 2 months (for example 28-56 days between doses), starting at 2 months of age, and followed by a toddler dose at 12-18 months of age. In an embodiment, said multiple dose schedule consists of a series of 3 doses separated by an interval of about 1 month to about 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more boosts are given at intervals that range from about 2 to about 24 weeks, preferably with a dosing interval of 4-8 weeks.

In an embodiment, a prime dose is given at day 0 and a boost is given about 3 months later.

7. The Invention Also Provides the Following Embodiments as Defined in the Following Numbered Paragraphs 1 to 89

1. A method of making a Streptococcus pneumoniae serotype 3 glycoconjugate, comprising the steps of:
(a) reacting an isolated Streptococcus pneumoniae serotype 3 capsular polysaccharide with 1,1′-carbonyldiimidazole (CDI) or 1,1′-Carbonyl-di-(1,2,4-triazole) (CDT) in an aprotic solvent;
(b) reacting the activated polysaccharide of step (a) with a carrier protein in an aprotic solvent to form a glycoconjugate.
2. The method of paragraph 1 wherein, the isolated polysaccharide is sized before the activation step (a).
3. The method of paragraph 2 wherein, the isolated polysaccharide is sized to a weight average molecular weight between 100 kDa and 200 kDa.
4. The method of any one of paragraphs 1-3 wherein, the isolated polysaccharide is reacted with 1,1′-carbonyldiimidazole (CDI).
5. The method of any one of paragraphs 1-3 wherein, the isolated polysaccharide is reacted with 1,1′-Carbonyl-di-(1,2,4-triazole) (CDT).
6. The method of any one of paragraphs 1-5 wherein, step a) comprises reacting the polysaccharide with an amount of CDI or CDT that is between 0.01-10 molar equivalent to the amount of serotype 3 capsular polysaccharide present in the reaction mixture.
7. The method of any one of paragraphs 1-6 wherein, the activating reaction a) is carried out in a solution consisting essentially of dimethylsulphoxide (DMSO) or dimethylformamide (DMF).
8. The method of any one of paragraphs 1-6 wherein, the activating reaction a) is carried out in a solution consisting essentially of dimethylsulphoxide (DMSO).
9. The method of any one of paragraphs 1-6 wherein, the activating reaction a) is carried out in an aprotic solvent comprising 0.1% to 1% water.
10. The method of any one of paragraphs 1-6 wherein, the activating reaction a) is carried out in DMSO comprising 0.1% to 1% water.
11. The method of any one of paragraphs 1-10 wherein, the activating reaction a) is followed by the addition of water.
12. The method of paragraph 11 wherein water is added to bring the total water content in the mixture to between about 1% to about 10% (v/v).
13. The method of any one of paragraphs 1-12 wherein, the conjugation reaction b) is carried out in a solution consisting essentially of dimethylsulphoxide (DMSO) or dimethylformamide (DMF).
14. The method of any one of paragraphs 1-12 wherein, the conjugation reaction b) is carried out in a solution consisting essentially of dimethylsulphoxide (DMSO).
15. The method of any one of paragraphs 1-12 wherein, the conjugation reaction b) is carried out in DMSO comprising about 0.1% to about 10% v/v water.
16. The method of any one of paragraphs 1-15 wherein the initial input ratio (weight by weight) of activated serotype 3 capsular polysaccharide to carrier protein at step b) is between 4:1 and 0.1:1.
17. The method of any one of paragraphs 1-16 wherein a weak organic base is added to the reaction mixture after the activating reaction a) but before the conjugation reaction b).
18. The method of paragraph 17 wherein the weak organic base is added to the reaction mixture before the carrier protein is introduced.
18. The method of paragraph 17 wherein the weak organic base is added to the reaction mixture after the carrier protein is introduced.
19. The method of any one of paragraphs 17-18 wherein said weak organic base is selected from alkanamines, imidazole, triazole, pyridine, histidine and guanidine.
20. The method of any one of paragraphs 17-19 wherein said organic base is added with an amount between 0.1-25 times (weight by weight) of the amount of polysaccharide to be activated.
21. The method of any one of paragraphs 1-20 wherein following the conjugation reaction b) unconjugated reactive sites of the activated polysaccharide are hydrolyzed.
22. The method of paragraph 21 wherein unconjugated reactive sites are hydrolyzed by addition to the conjugation solution of an aqueous solution.
23. The method of paragraph 21 wherein unconjugated reactive sites are hydrolyzed by addition to the conjugation solution of an aqueous buffered solution.
24. The method of any one of paragraphs 1-23 further comprising the step of purifying the glycoconjugate after it is produced.
25. A method of making a Streptococcus pneumoniae serotype 3 glycoconjugate, comprising the steps of:
(a) reacting an isolated Streptococcus pneumoniae serotype 3 capsular polysaccharide with an oxidizing agent;
(b) compounding the activated polysaccharide of step (a) with a carrier protein; and
(c) reacting the compounded activated polysaccharide and carrier protein with a reducing agent to form a glycoconjugate.
26. The method of paragraph 25 wherein, the isolated polysaccharide is sized before the activation step (a).
27. The method of paragraph 25 wherein, the isolated polysaccharide is sized before the activation step (a) to a weight average molecular weight between 100 kDa and 200 kDa.
28. A method of making a Streptococcus pneumoniae serotype 3 glycoconjugate, comprising the steps of:
(a) reacting an isolated Streptococcus pneumoniae serotype 3 capsular polysaccharide with an oxidizing agent;
(a′) quenching the oxidation reaction by addition of a quenching agent;
(b) compounding the activated polysaccharide of step (a) or (a′) with a carrier protein; and
(c) reacting the compounded activated polysaccharide and carrier protein with a reducing agent to form a glycoconjugate.
29. The method of paragraph 28 wherein, the isolated polysaccharide is sized before the activation step (a).
30. The method of paragraph 28 wherein, the isolated polysaccharide is sized before the activation step (a) to a weight average molecular weight between 100 kDa and 200 kDa.
31. The method of any one of paragraphs 25-30 wherein the oxidizing agent is periodate.
32. The method of any one of paragraphs 25-30 wherein the oxidizing agent is periodic acid.
33. The method of any one of paragraphs 25-31 wherein step a) comprises reacting the polysaccharide with 0.01-2 molar equivalents of periodate.
34. The method of any one of paragraphs 25-33 wherein the degree of oxidation of the activated serotype 3 polysaccharide is between 2 and 30.
35. The method of any one of paragraphs 25-33 wherein the degree of oxidation of the activated serotype 3 polysaccharide is between 11 to 19.
36. The method of any one of paragraphs 25-33 wherein the degree of oxidation of the activated serotype 3 polysaccharide is about 15.
37. The method of any one of paragraphs 25-36 wherein the initial input ratio (weight by weight) of activated serotype 3 capsular polysaccharide to carrier protein at step b) is between 4:1 and 0.1:1.
38. The method of any one of paragraphs 25-36 wherein the initial input ratio (weight by weight) of activated serotype 3 capsular polysaccharide to carrier protein is between 1.5:1 and 0.5:1.
39. The method of any one of paragraphs 25-38 wherein the reduction reaction (c) is carried out in aqueous solvent.
40. The method of any one of paragraphs 25-38 wherein the reduction reaction (c) is carried out in aprotic solvent.
41. The method of any one of paragraphs 25-38 wherein the reduction reaction (c) is carried out in a solution consisting essentially of dimethylsulphoxide (DMSO).
42. The method of any one of paragraphs 25-41 wherein the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMe′PrN-BH₃, benzylamine-BH₃ or 5-ethyl-2-methylpyridine borane (PEMB).
43. The method of any one of paragraphs 25-41 wherein the reducing agent is sodium cyanoborohydride.
44. The method of any one of paragraphs 25-43 wherein between 0.2 and 20 molar equivalents of reducing agent is used in step c).
45. The method of any one of paragraphs 25-43 wherein between 0.5 and 3 molar equivalents of reducing agent is used in step c).
46. The method of any one of paragraphs 25-45 wherein, the product of step c) is reacted with 1 to 20 molar equivalents of sodium borohydride for 15 mins-15 hrs.
47. The method of any one of paragraphs 25-46 further comprising the step of purifying the glycoconjugate after it is produced.
48. A Streptococcus pneumoniae serotype 3 glycoconjugate produced according to any one of the methods of paragraphs 1 to 47.
49. The glycoconjugate of paragraph 48 comprising a serotype 3 capsular polysaccharide wherein the weight average molecular weight (Mw) of said polysaccharide before conjugation is between 25 kDa and 200 kDa.
50. The glycoconjugate of paragraph 48 comprising a serotype 3 capsular polysaccharide wherein the weight average molecular weight (Mw) of said polysaccharide before conjugation is between 50 kDa and 175 kDa.
51. The glycoconjugate of paragraph 48 comprising a serotype 3 capsular polysaccharide wherein the weight average molecular weight (Mw) of said polysaccharide before conjugation is between 100 kDa and 200 kDa.
52. The glycoconjugate of paragraph 48 comprising a serotype 3 capsular polysaccharide wherein the weight average molecular weight (Mw) of said polysaccharide before conjugation is between 120 kDa and 180 kDa.
53. The glycoconjugate of paragraph 48 comprising a serotype 3 capsular polysaccharide wherein the weight average molecular weight (Mw) of said polysaccharide before conjugation is between 130 kDa and 170 kDa.
54. The glycoconjugate of paragraph 48 comprising a serotype 3 capsular polysaccharide wherein the weight average molecular weight (Mw) of said polysaccharide before conjugation is between 140 kDa and 160 kDa.
55. The glycoconjugate of paragraph 48 comprising a serotype 3 capsular polysaccharide wherein the weight average molecular weight (Mw) of said polysaccharide before conjugation is about 150 kDa.
56. The glycoconjugate of any one of paragraphs 48-55 having a weight average molecular weight (Mw) of between 250 kDa and 15,000 kDa.
57. The glycoconjugate of any one of paragraphs 48-55 having a weight average molecular weight (Mw) of between 1,000 kDa and 5,000 kDa.
58. The glycoconjugate of any one of paragraphs 48-55 having a weight average molecular weight (Mw) of between 1,000 kDa and 4,000 kDa.
59. The glycoconjugate of any one of paragraphs 48-55 having a weight average molecular weight (Mw) of between 2,250 kDa and 3,500 kDa.
60. The glycoconjugate of any one of paragraphs 48-59 wherein the degree of conjugation of said glycoconjugate is between 2 and 15.
61. The glycoconjugate of any one of paragraphs 48-60 wherein the saccharide to carrier protein ratio (w/w) is between 0.5 and 1.5.
61. The glycoconjugate of any one of paragraphs 48-60 wherein the saccharide to carrier protein ratio (w/w) is between 0.9 and 1.1.
62. The glycoconjugate of any one of paragraphs 48-61 wherein said carrier protein is CRM₁₉₇.
63. The glycoconjugate of any one of paragraphs 48-61 wherein said carrier protein is SCP.
64. The glycoconjugate of any one of paragraphs 48-61 wherein said carrier protein is an enzymatically inactive SCP.
65. The glycoconjugate of any one of paragraphs 48-61 wherein said carrier protein is an enzymatically inactive SCP from GBS (SCPB).
66. The glycoconjugate of any one of paragraphs 48-61 wherein said carrier protein is a fragment of an SCPB.
67. The glycoconjugate of any one of paragraphs 48-61 wherein said carrier protein is a fragment of an SCP which comprises the protease domain, the protease-associated domain (PA domain) and the three fibronectin type III (Fn) domains but does not comprise the export signal presequence, the pro-sequence and the cell wall anchor domain.
68. The glycoconjugate of any one of paragraphs 48-61 wherein said carrier protein is an enzymatically inactive fragment of an SCP. In an embodiment, said enzymatically inactive fragment of SCP comprises the protease domain, the protease-associated domain (PA domain) and the three fibronectin type III (Fn) domains but does not comprise the export signal presequence, the pro-sequence and the cell wall anchor domain.
69. The glycoconjugate of any one of paragraphs 48-61 wherein said carrier protein is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence and wherein said replacement is selected from the group consisting of D130A, H193A, N295A and S512A where the numbers indicate the amino acid residue position in the peptidase according to the numbering of SEQ ID NO: 1 of WO00/34487.
70. The glycoconjugate of any one of paragraphs 48-61 wherein said carrier protein is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence wherein said at least two amino acids replacements are D130A and S512A where the numbers indicate the amino acid residue position in the peptidase according to the numbering of SEQ ID NO: 1 of WO00/34487.
71. The glycoconjugate of any one of paragraphs 48-61 wherein said carrier protein is an enzymatically inactive fragment of SCP consisting of a polypeptide having at least 95% identity with SEQ ID NO: 41.
72. The glycoconjugate of any one of paragraphs 48-61 wherein said carrier protein is an enzymatically inactive fragment of SCP consisting of a polypeptide having at least 95% identity with SEQ ID NO: 42.
73. The glycoconjugate of any one of paragraphs 48-61 wherein said carrier protein is an enzymatically inactive fragment of SCP which consists of SEQ ID NO: 41.
74. The glycoconjugate of any one of paragraphs 48-61 wherein said carrier protein is an enzymatically inactive fragment of SCP which consists of SEQ ID NO: 42.
75. An immunogenic composition comprising a Streptococcus pneumoniae serotype 3 glycoconjugate of any one of paragraphs 48 to 74.
76. The immunogenic composition of paragraph 75 comprising from 1 to 25 glycoconjugates from different serotypes of S. pneumoniae.
77. The immunogenic composition of paragraph 75 comprising glycoconjugates from 7, 8, 9, 10, 11, 12, 13, 14, 15, 16, 17, 18, 19, 20, 21, 22, 23, 24 or 25 different serotypes of S. pneumoniae.
78. The immunogenic composition of paragraph 75 which is a 15-valent pneumococcal conjugate composition.
79. The immunogenic composition of paragraph 75 which is a 20-valent pneumococcal conjugate composition.
80. The immunogenic composition of any one of paragraphs 75-79 further comprising glycoconjugates from S. pneumoniae serotypes 4, 6B, 9V, 14, 18C, 19F and 23F.
81. The immunogenic composition of paragraph 80 comprising in addition glycoconjugates from S. pneumoniae serotypes 1, 5 and 7F.
82. The immunogenic composition of paragraph 81 comprising in addition glycoconjugates from S. pneumoniae serotypes 6A and 19A.
83. The immunogenic composition of paragraph 82 comprising in addition glycoconjugates from S. pneumoniae serotype 22F and 33F.
84. The immunogenic composition of paragraph 83 comprising in addition glycoconjugates from S. pneumoniae serotypes 8, 10A, 11A, 12F and 15B.
85. The immunogenic composition of paragraph 75 further comprising glycoconjugates from S. pneumoniae serotypes 1, 4, 5, 6A, 6B, 7F, 9V, 14, 18C, 19A, 19F and 23F, wherein said immunogenic composition is a 13-valent pneumococcal conjugate composition.
86. The immunogenic composition of paragraph 75 further comprising glycoconjugates from S. pneumoniae serotypes 1, 4, 5, 6A, 6B, 7F, 9V, 14, 18C, 19A, 19F, 22F, 23F and 33F wherein said immunogenic composition is a 15-valent pneumococcal conjugate composition.
87. The immunogenic composition of paragraph 75 further comprising glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F, wherein said immunogenic composition is a 20-valent pneumococcal conjugate composition.
88. The immunogenic composition of paragraph 75 further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein said immunogenic composition is a 25-valent pneumococcal conjugate composition.
89. The immunogenic composition of paragraph 75 further comprising glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38, wherein said immunogenic composition is a 25-valent pneumococcal conjugate composition.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of and” consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLE

### Example 1. Preparation of Serotype 3 Glycoconjugate Using Reductive Amination in Aqueous Buffer (RAC/Aq.)

### 1. Hydrolysis

The native polysaccharide was hydrolyzed to reduce the molecular weight prior to activation. A calculated volume of 2M acetic acid was added to the polysaccharide solution to achieve a final polysaccharide concentration of 2.0±0.2 g/L and a final acetic acid concentration of 0.2M. The diluted polysaccharide solution was heated to 85±5° C. The hydrolysis reaction was maintained for certain time depending on the target polysaccharide Mw. At the end of reaction, the mixture was cooled to 23±2° C.

### 2. Oxidation

For the oxidation reaction, 1M magnesium chloride was added to the reaction solution to a final concentration of 0.10M. Periodic acid was then added to the polysaccharide solution to initiate the oxidation reaction (added as a 50 mg/mL solution in WFI). The required molar equivalent of periodic acid was selected based on the target Degree of Oxidation (DO). The target range for the DO was 5.0±3.0. The oxidation reaction time was 20±4 hours, at 23±2° C.

### 3. Purification of the Activated Polysaccharide

The activated polysaccharide was purified by tangential flow filtration against WFI. The diafiltration was performed using polyethersulfone (PES) flat sheet membranes with a molecular weight cut-off (MWCO) of 100 kDa. Once the diafiltration was complete, the activated polysaccharide was characterized, by (i) saccharide concentration by colorimetric assay; (ii) aldehyde concentration by colorimetric assay; (iii) degree of oxidation; and (iv) molecular weight by SEC-MALLS. The pH of purified saccharide was adjusted to 6.3±0.3. Protein (CRM₁₉₇, TT or SCP) was then added to a predetermined ratio. The mixture was then shell frozen and lyophilized to dry.

### 4. Conjugation Reaction

The lyophilized activated polysaccharide and protein were reconstituted in 0.1M sodium phosphate buffer. After the reconstitution was complete, the pH was adjusted to a final pH of 6.5±0.2 using 1N hydrochloric acid or 1N sodium hydroxide. To initiate the conjugation reaction, predetermined molar equivalent of sodium cyanoborohydride was added to the reaction mixture. The conjugation proceeded for a period of 40±4 hours at 30±2° C. with continuous mixing at 100±10 rpm.

### 5. Dilution and Capping Reaction

After the conjugation reaction time was complete, the reaction solution was cooled to 23±2° C. and diluted by a factor of 0.5-1.0 times the reaction volume with 0.9% NaCl buffer, 1 molar equivalent of sodium borohydride was then added to the mixture. The capping reaction proceeded for a period of 3-6 hours at 23±2° C. with continuous mixing at 100±10 rpm.

### 6. Purification of Conjugate

The diluted conjugate solution was passed through a 5 μm filter, and diafiltration was performed using 5 mM succinate/0.9% saline (pH 6.0) as the medium. After the diafiltration was completed, the conjugate retentate was filtered through a 0.45 um/0.22 μm filter.

### Example 2. Preparation of Serotype 3 Glycoconjugate Using Reductive Amination in Dimethylsulfoxide (RAC/DMSO)

### 1. Hydrolysis and Oxidation

Polysaccharide hydrolysis, activation and diafiltration were performed in the same manner as described for above aqueous based conjugation. The required molar equivalent of sodium periodate was selected based on the target DO. The target range for the DO is 15.0±4.0. The oxidation reaction time is 20±4 hours, at 23±2° C.

### 2. Compounding and Lyophilization

The activated polysaccharide was compounded with sucrose to a ratio of 25-100 grams of sucrose per gram of activated polysaccharide, preferably at a ratio of 40-60 grams of sucrose per gram of activated polysaccharide. The compounded mixture was then lyophilized. Calculated amount of carrier proteins (CRM₁₉₇, TT or SCP) were shell-frozen and lyophilized separately.

### 3. Conjugating and Capping

Lyophilized activated polysaccharide was reconstituted in anhydrous dimethyl sulfoxide (DMSO), an equal amount of anhydrous DMSO was used to reconstitute the carrier protein.

Reconstituted activated polysaccharide was combined with reconstituted carrier protein in the reaction vessel, followed by mixing thoroughly to obtain a clear solution before initiating the conjugation with sodium cyanoborohydride. The final polysaccharide concentration in reaction solution was approximately 1 g/L. Conjugation was initiated by adding 0.5-2.0 MEq of sodium cyanoborohydride to the reaction mixture and incubating at 23±2° C. for 20-48 hrs. The conjugation reaction was terminated by adding 2 MEq of sodium borohydride (NaBH4) to cap unreacted aldehydes. This capping reaction continued at 23±2° C. for 3±1 hrs.

### 4. Purification

The conjugate solution was diluted 1:10 with chilled 5 mM succinate-0.9% saline (pH 6.0) in preparation for purification by tangential flow filtration using 100-300K MWCO membranes. The diafiltration was then performed using 5 mM succinate/0.9% saline (pH 6.0) as the medium. After the diafiltration was completed, the conjugate retentate was transferred through a 0.22 μm filter. The conjugate was diluted further with 5 mM succinate/0.9% saline (pH 6), to a target saccharide concentration of approximately 0.5 mg/mL. Alternatively, the conjugate was purified using 20 mM Histidine-0.9% saline (pH 6.5) by tangential flow filtration using 100-300K MWCO membranes. Final 0.22 μm filtration step was completed to obtain the immunogenic conjugate.

| | RAC/DMSO | RAC/DMSO | RAC/DMSO | RAC/Aqueous |
|---|---|---|---|---|
| | Protein: CRM | Protein: TT | Protein: SCP | Protein: CRM |
| Poly MW (kDa) | 166 | 199 | 199 | 140 |
| Degree of | 14 | 14 | 14 | 5 |
| Oxidation (DO) | | | | |
| Saccharide/Protein Ratio | 0.9 | 0.9 | 1.0 | 1.0 |
| % Free Saccharide | <5% | <5% | <5% | 8.5% |
| Conjugate MW by | 2670 | 3962 | 4760 | 1530 |
| SEC-MALLS, kDa | | | | |

### Example 3. Effect of the Size of the Polysaccharide for Serotype 3 Glycoconjugate

The opsonophagocytic activity (OPA) titers for Serotype 3-CRM₁₉₇ conjugates in mice comprising polysaccharide of different size were determined under standard conditions.

Sized Serotype 3 polysaccharides (˜25, 150, or 250 kDa) conjugated to CRM₁₉₇ using the either RAC/Aqueous (see example 1) or RAC/DMSO (see example 2) conjugation to -CRM₁₉₇ was used to vaccinate animals in the presence of adjuvant (see attributes of the tested conjugates at Table 2).

TABLE 2
Attributes of Pn3 Conjugates for evaluation of effect size of the
Polysaccharide

| | | RAC/ | RAC/ | RAC/ | RAC/ | RAC/ |
|---|---|---|---|---|---|---|
| | RAC/Aq. | Aq. | Aq. | DMSO | DMSO | DMSO |
| | High | Medium | Low | High | Medium | Low |
| | MW | MW | MW | MW | MW | MW |
| Activated | 250 | 150 | 25 | 250 | 150 | 25 |
| Polysaccharide | | | | | | |
| MW, kDa | | | | | | |
| Conjugate MW | 2467 | 1278 | 1972 | 3123 | 2670 | 1790 |
| (kDa) | | | | | | |
| Degree of | 3.9 | 10 | 7 | 14 | 14 | 13 |
| Activation | | | | | | |
| SPR Ratio | 0.9 | 0.8 | 1.1 | 1 | 0.94 | 0.9 |
| Free Saccharide, | <5 | 9 | <5 | 4.5 | 7 | 13 |
| % | | | | | | |

MW: molecular weight;
SPR: Saccharide to protein ratio

Groups of twenty-five 6-8 weeks old female Swiss Webster mice were immunized (250 μL) with 0.01 μg/ml, 0.1 μg/ml, or 1 μg/ml of test conjugates via the subcutaneous route on week 0. The mice were boosted with the same dose of conjugate on week 3 and then bled at week 5. Each vaccination was formulated with 100 μg/dose of AlPO₄ as an adjuvant. All preclinical immunogenicity studies were powered to detect a 4 to 5-fold difference in OPA titers using 25 mice per group. Whole blood was collected from mice two weeks after the second vaccination (Week 5, PD 2) and sera used for analyses. Serotype-specific OPAs were performed on week 5 sera samples.

Opsonophagocytic activity (OPA) assays are used to measure functional antibodies in murine sera specific for S. pneumonia serotype 3. Test serum is set up in assay reactions that measure the ability of capsular polysaccharide specific immunoglobulin to opsonize bacteria, trigger complement deposition, thereby facilitating phagocytosis and killing of bacteria by phagocytes. The OPA titer is defined as the reciprocal dilution that results in a 50% reduction in bacterial count over control wells without test serum. The OPA titer is interpolated from the two dilutions that encompass this 50% killing cut-off.

OPA procedures were based on methods described in Hu et al. (2005) Clin Diagn Lab Immunol 12 (2):287-295 with the following modifications. Test serum was serially diluted 2.5-fold and added to microtiter assay plates. Live serotype 3 target bacterial strains were added to the wells and the plates were shaken at 25° C. for 30 minutes. Differentiated HL-60 cells (phagocytes) and baby rabbit serum (3- to 4-week old, PEL-FREEZ®, 12% final concentration) were added to the wells, and the plates were shaken at 37° C. for 45 minutes. To terminate the reaction, 80 μL of 0.9% NaCl was added to all wells, mixed, and a 10 μL aliquot were transferred to the wells of MULTISCREEN® HTS HV filter plates (MILLIPORE®) containing 200 μL of water. Liquid was filtered through the plates under vacuum, and 150 μL of HYSOY® medium was added to each well and filtered through. The filter plates were then incubated at 37° C., 5% CO₂ overnight and were then fixed with Destain Solution (Bio-Rad Laboratories, Inc., Hercules, Calif.). The plates were then stained with Coomassie Blue and destained once. Colonies were imaged and enumerated on a Cellular Technology Limited (CTL) (Shaker Heights, Ohio) IMMUNOSPOT® Analyzer. Raw colony counts were used to plot kill curves and calculate OPA titers.

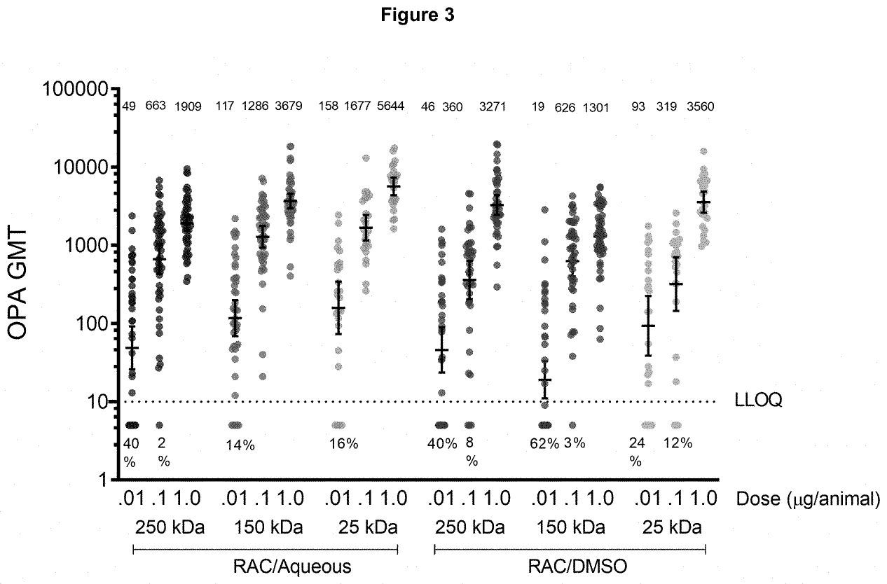

OPA titers (geometric mean titer (GMT) with 95% confidence interval (CI)) at five weeks at different doses are shown in Table 3. The results are presented in FIG. 3.

TABLE 3
OPA titers following vaccination with sized Serotype 3 polysaccharide
conjugated to -CRM₁₉₇. Sized (~25, 150, or 250 kDa) Serotype 3
conjugated to CRM₁₉₇ using the either Rac/aqueous or RAC/DMSO
was used to vaccinate animals in the presence of adjuvant. Female
Swiss-Webster mice, 6-8 weeks old; Doses: 0.01; 0.1 and 1 μg/ml +
AIPO₄; Vaccinate: 0 and 3 wk.; exsang wk. 5 Readout: OPA

| | | 0.01 | 0.1 | 1 |
|---|---|---|---|---|
| 250 kDa (RAC/Aq.) | Mean | 49 | 663 | 1909 |
| | Total # of mice | 47 | 49 | 49 |
| | # of non-responders | 19 | 1 | 0 |
| | % of non-responders | 40 | 2 | 0 |
| 150 kDa (RAC/Aq.) | Mean | 117 | 1286 | 3679 |
| | Total # of mice | 44 | 49 | 49 |
| | # of non-responders | 6 | 0 | 0 |
| | % of non-responders | 14 | 0 | 0 |
| 25 kDa (RAC/Aq.) | Mean | 158 | 1677 | 5644 |
| | Total # of mice | 25 | 25 | 25 |
| | # of non-responders | 4 | 0 | 0 |
| | % of non-responders | 16 | 0 | 0 |
| 250 kDa (RAC/DMSO) | Mean | 46 | 360 | 3271 |
| | Total # of mice | 40 | 39 | 40 |
| | # of non-responders | 16 | 3 | 0 |
| | % of non-responders | 40 | 8 | 0 |
| 150 kDa (RAC/DMSO) | Mean | 19 | 626 | 1285 |
| | Total # of mice | 47 | 40 | 47 |
| | # of non-responders | 29 | 1 | 0 |
| | % of non-responders | 62 | 3 | 0 |
| 25 kDa (RAC/DMSO) | Mean | 93 | 319 | 3560 |
| | Total # of mice | 25 | 25 | 25 |
| | # of non-responders | 6 | 3 | 0 |
| | % of non-responders | 24 | 12 | 0 |

The data of Table 3 and FIG. 3 indicate that the serotype 3 conjugates elicited dose dependent OPA titers in a murine immunogenicity model. As shown in Table 3, serotype 3 conjugates with RAC/Aqueous chemistry induced higher OPA GMT at all doses as the polysaccharide size decreased. For the RAC/DMSO chemistry there were less non-responders at the 0.01 μg dose with the small polysaccharide size.

### Example 4. Effect of Degree of Oxidation (DO)/Degree of Activation (DoA) of the Polysaccharide for Serotype 3 Glycoconjugate

The opsonophagocytic activity (OPA) titers for Serotype 3-CRM₁₉₇ conjugates in mice generated using different degree of oxidation were determined under standard conditions.

Sized Serotype 3 polysaccharides (˜120-170 kDa) conjugated to CRM₁₉₇ using the either RAC/Aqueous (see example 1) or RAC/DMSO (see example 2) conjugation to -CRM₁₉₇ was used to vaccinate animals in the presence of adjuvant (see attributes of the tested conjugates at Table 4).

TABLE 4
Attributes of Pn3 Conjugates for Evaluation of Degree of Oxidation

| | RAC/DMSO | RAC/DMSO | RAC/Aqueous | RAC/Aqueous |
|---|---|---|---|---|
| | Low DO | High DO | Low DO | High DO |
| Activated | 156 | 166 | 140 | 123 |
| Polysaccharide | | | | |
| MW, kDa | | | | |
| Conjugate MW (kDa) | 1580 | 2670 | 1530 | 1278 |
| Degree of | 7 | 14 | 5 | 10 |
| Activation (DoA) | | | | |
| SPR Ratio | 1 | 0.94 | 1 | 0.8 |
| Free Saccharide, % | <5 | 7 | 8.5 | 9 |

MW: molecular weight;
SPR: Saccharide to protein ratio

Groups of twenty-five 6-8 weeks old female Swiss Webster mice were immunized (250 μL) with 0.01 μg/ml, 0.1 μg/ml, or 1 μg/ml of test conjugates via the subcutaneous route on week 0. The mice were boosted with the same dose of conjugate on week 3 and then bled at week 5. Serotype-specific OPAs were performed on week 5 sera samples.

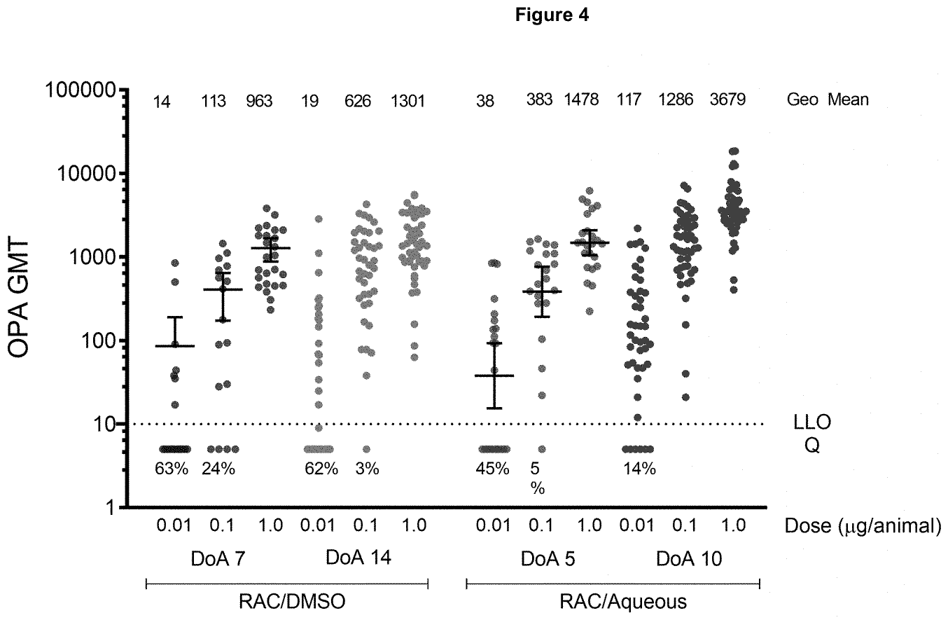

OPA was conducted as described at Example 3. The results are presented at table 5 and FIG. 4.

TABLE 5
OPA titers following vaccination with Serotype 3 antigen with
variable degree of oxidation (DO) - conjugated to -CRM. Serotype
3 conjugated to CRM was used to vaccinate animals in the presence
of adjuvant. Female Swiss-Webster mice, 6-8 weeks old; Doses:
0.01; 0.1 and 1 μg/ml + AIPO₄; Vaccinate: 0 and 3 wk.; exsang
wk. 5 Readout: OPA

| | | 0.01 | 0.1 | 1 |
|---|---|---|---|---|
| DO 7 (RAC/DMSO) | Mean | 14 | 113 | 963 |
| | Total # of mice | 19 | 17 | 25 |
| | # of non-responders | 12 | 4 | 0 |
| | % of non-responders | 63 | 24 | 0 |
| DO 14 (RAC/DMSO) | Mean | 19 | 626 | 1301 |
| | Total # of mice | 47 | 40 | 48 |
| | # of non-responders | 29 | 1 | 0 |
| | % of non-responders | 62 | 3 | 0 |
| DO 5 (RAC/Aq.) | Mean | 38 | 383 | 1478 |
| | Total # of mice | 22 | 21 | 24 |
| | # of non-responders | 10 | 1 | 0 |
| | % of non-responders | 45 | 5 | 0 |
| DO 10 (RAC/Aq.) | Mean | 117 | 1286 | 3679 |
| | Total # of mice | 44 | 49 | 49 |
| | # of non-responders | 6 | 0 | 0 |
| | % of non-responders | 14 | 0 | 0 |

The data of Table 5 and FIG. 4 indicate that the Serotype 3 conjugates elicited dose dependent OPA titers in a murine immunogenicity model. As shown in Table 5, serotype 3 conjugates with RAC/Aqueous or RAC/DMSO chemistry induced higher OPA GMT at all doses with a higher DO.

### Example 5. Effect of the Carrier Protein on Immunogenicity for Serotype 3 Glycoconjugates

The opsonophagocytic activity (OPA) titers for Serotype 3 conjugates to -CRM₁₉₇, -SCP, or Tetanus toxoid in mice were determined under standard conditions. Reductive Amination in DMSO (RAC/DMSO) was used (see example 2).

Sized Serotype 3 polysaccharides (˜160-250 kDa) conjugated to different protein carrier was used to vaccinate animals in the presence of adjuvant (see attributes of the tested conjugates at Table 6).

TABLE 6
Attributes of Pn3 Conjugates for Chemistry Evaluation
(RAC/DMSO)

| | CRM₁₉₇ | TT | SCP |
|---|---|---|---|
| Activated | 166 | 199 | 199 |
| Polysaccharide | | | |
| MW, kDa | | | |
| Conjugate MW | 2670 | 3962 | 4760 |
| (kDa) | | | |
| Degree of | 14 | 14 | 14 |
| Activation | | | |
| SPR Ratio | 0.94 | 0.92 | 1 |
| Free | 7 | <5 | <5 |
| Saccharide, % | | | |

MW: molecular weight;
SPR: Saccharide to protein ratio

Groups of twenty-five 6-8 weeks old female Swiss Webster mice were immunized (250 μL) with 0.01 μg/ml, 0.1 μg/ml, or 1 μg/ml of test conjugates via the subcutaneous route on week 0. The mice were boosted with the same dose of conjugate on week 3 and then bled at week 5. Serotype-specific OPAs were performed on week 5 sera samples.

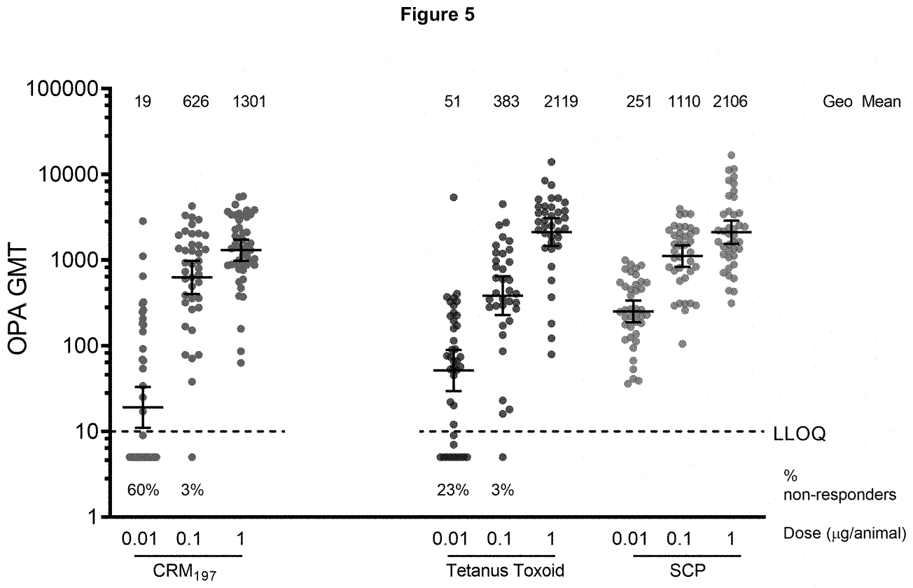

The results are presented at table 7 and FIG. 5.

TABLE 7
OPA titers following vaccination Serotype 3 antigen conjugate to -
CRM₁₉₇, -SCP, or Tetanus toxoid. Conjugated Serotype 3 was
used to vaccinate animals in the presence of adjuvant. Female
Swiss-Webster mice, 6-8 weeks old; Doses: 0.01; 0.1 and 1 μg/ml +
AIPO₄; Vaccinate: 0 and 3 wk.; exsang wk. 5 Readout: OPA

| | | 0.01 | 0.1 | 1 |
|---|---|---|---|---|
| Tetanus Toxoid | Mean | 51 | 383 | 2119 |
| (RAC/DMSO) | Total # of mice | 40 | 35 | 37 |
| | # of non-responders | 9 | 1 | 0 |
| | % of non-responders | 23 | 3 | 0 |
| SCP | Mean | 251 | 1110 | 2106 |
| (RAC/DMSO) | Total # of mice | 40 | 38 | 40 |
| | # of non-responders | 0 | 0 | 0 |
| | % of non-responders | 0 | 0 | 0 |
| CRM197 | Mean | 19 | 626 | 1301 |
| (RAC/DMSO) | Total # of mice | 47 | 40 | 48 |
| | # of non-responders | 28 | 1 | 0 |
| | % of non-responders | 60 | 3 | 0 |

The data of Table 7 and FIG. 5 indicate that the serotype 3 conjugates elicited dose dependent OPA titers in a murine immunogenicity model. As shown in Table 7, serotype 3 conjugated to SCP induced higher OPA GMT and all mice responded with measurable OPA titiers (0% non-responders) even at the low dose.

As depicted here, only SCP truly enhanced the percentage of mice responding to vaccination at the 0.01 μg/ml dose. TT elicited responses generally lower than CRM₁₉₇.

### Example 6: Conjugation of Serotype 3 Capsular Polysaccharide Prepared Using CDI Chemistry

### 1. Hydrolysis, Compounding and Lyophilization

Polysaccharide hydrolysis, targeting the Mw of about 40 KDa or about 200 KDa was performed in the same manner as described for above aqueous based reductive amination conjugation (Example 1). The sized polysaccharide was purified through a PES (PolyEtherSulfone)-based membrane using water. The purified polysaccharide was then mixed with 1-50× (by weight) of imidazole. Optionally, the pH of the resulting mixture was adjusted between 3.0-6.0. The mixture was then lyophilized. Calculated amount of carrier protein (CRM₁₉₇) was buffer exchanged into an 5 mM imidazole-based buffer system, shell-frozen and lyophilized separately.

### 2. Polysaccharide Reconstitution and CDI Activation

Polysaccharide was reconstituted in anhydrous DMSO, moisture level was adjusted within 0.1-1% by adding water. Activation was initiated by adding freshly prepared CDI (100 mg/mL in DMSO) solution at temperature between 23-50° C. The reaction was continued for 3 hrs. Upon completion of CDI activation, water was added to bring the total water content in the mixture to about 2% (v/v) and the mixture was stirred for another 0.5-1 hr. Imidazole solution in DMSO was then added to the mixture.

### 3. Conjugation

The lyophilized CRM₁₉₇ was reconstituted in DMSO and was then transferred to the activated polysaccharide/DMSO solution. The reaction was incubated at 23° C. for 19±3 hrs with moderate mixing.

### 4. Hydrolysis

The conjugation solution was then diluted into 0.2M sodium acetate pH 3.6. The pH was adjusted, if needed, using NaOH or HCl to a pH of 4.0±0.2. The hydrolysis reaction proceeded for 4±1 hrs at 23° C. At this step, unconjugated reactive sites were hydrolyzed. At the end of the hydrolysis step, the conjugate reaction mixture was neutralized with 1M phosphate dibasic solution to pH 6.0±0.2. The neutralized conjugate solution then passed through a 5 μm pre-filter.

### 5. Purification of Conjugation

Concentration and diafiltration were carried out using 100K MWCO regenerated cellulose membranes. Diafiltration was performed against 30-fold diavolume of 5 mM succinate-0.9% saline, pH 6.0. The conjugate was then filtered through a 0.45 μm/0.22 μm filter.

TABLE 8
summarizes the results from CDI based conjugation

| | Conjugate 1 | Conjugate 2 |
|---|---|---|
| Poly MW (kDa) | 40 | 181 |
| Saccharide/Protein | 0.6 | 0.9 |
| Ratio | | |
| % Free | <5% | <5% |
| Saccharide | | |
| Conjugate MW by | 1570 | 4644 |
| SEC-MALLS, kDa | | |

### Example 7. Effect of Chemistry on Immunogenicity for Serotype 3 Glycoconjugates

The opsonophagocytic activity (OPA) titers for Serotype 3 conjugates to -CRM₁₉₇ in mice were determined under standard conditions. Different chemistries (Reductive Amination in aqueous (RAC/Aq.) see example 1, Reductive Amination in DMSO (RAC/DMSO) see example 2, eTEC linked glycoconjugates (eTEC) see WO2014/027302 or CDI chemistry (CDI) see example 6) were used to evaluate changes in OPA responses in mice.

Sized Serotype 3 polysaccharides (˜160-1100 kDa) conjugated to CRM₁₉₇ using different chemistries were used to vaccinate animals in the presence of adjuvant (see attributes of the tested conjugates at Table 9).

TABLE 9
Attribtues of Pn3 Conjugates for evaluation of
conjugation chemistry

| | RAC/Aq. | RAC/DMSO | eTEC | CDI |
|---|---|---|---|---|
| Activated | 250 | 234 | 1131 | 165 |
| Polysaccharide | | | | |
| MW, kDa | | | | |
| Conjugate MW | 2467 | 3123 | 2278 | 4406 |
| (kDa) | | | | |
| Degree of | 3.9 | 14 | 19 | 0.5 |
| Activation | | | | |
| SPR Ratio | 0.9 | 1 | 1.1 | 1 |
| Free Saccharide, % | <5 | 4.5 | 3 | <5 |

MW: molecular weight;
SPR: Saccharide to protein ratio

Groups of twenty-five 6-8 weeks old female Swiss Webster mice were immunized (250 μL) with 0.01 μg/ml, 0.1 μg/ml, or 1 μg/ml of test conjugates via the subcutaneous route on week 0. The mice were boosted with the same dose of conjugate on week 3 and then bled at week 5. Serotype-specific OPAs were performed on week 5 sera samples.

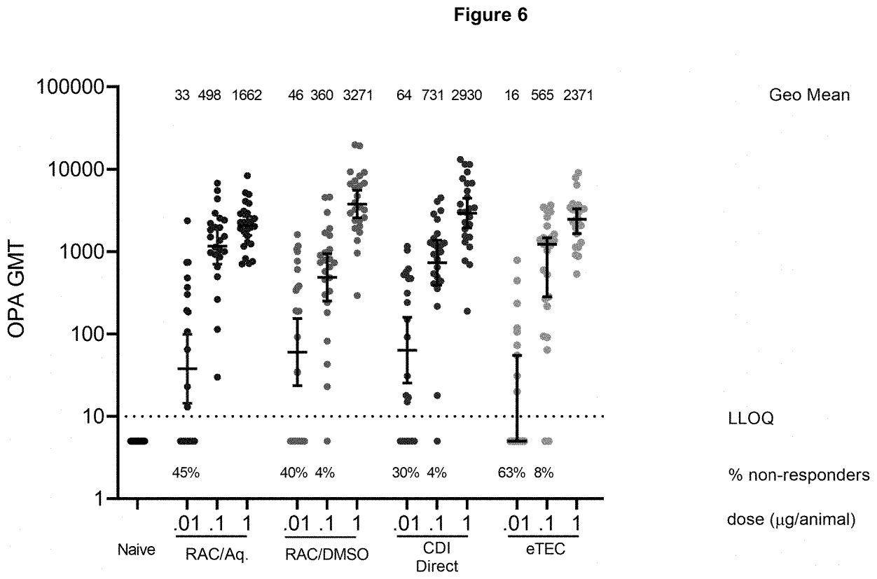

The results are presented at table 10 and FIG. 6.

TABLE 10
OPA titers following vaccination Serotype 3 antigen conjugate to -
CRM₁₉₇, using different chemistries: RAC/Aq., RAC/DMSO,
eTEC or CDI. Conjugated Serotype 3 was used to vaccinate
animals in the presence of adjuvant. Female Swiss-Webster mice,
6-8 weeks old; Doses: 0.01; 0.1 and 1 μg/ml + AIPO4; Vaccinate:
0 and 3 wk.; exsang wk. 5 Readout: OPA

| | | 0.01 | 0.1 | 1 |
|---|---|---|---|---|
| CRM₁₉₇ (RAC/Aq.) | Mean | 38 | 1165 | 2049 |
| | Total # of mice | 22 | 24 | 25 |
| | # of non-responders | 10 | 0 | 0 |
| | % of non-responders | 45 | 0 | 0 |
| CRM₁₉₇ | Mean | 60 | 486 | 3766 |
| (RAC/DMSO) | Total # of mice | 25 | 25 | 25 |
| | # of non-responders | 10 | 1 | 0 |
| | % of non-responders | 40 | 4 | 0 |
| CRM₁₉₇ (eTEC) | Mean | 16 | 565 | 2371 |
| | Total # of mice | 24 | 25 | 24 |
| | # of non-responders | 15 | 2 | 0 |
| | % of non-responders | 63 | 8 | 0 |
| CRM₁₉₇ (CDI) | Mean | 64 | 731 | 2930 |
| | Total # of mice | 23 | 25 | 25 |
| | # of non-responders | 7 | 1 | 0 |
| | % of non-responders | 30 | 4 | 0 |

The data of Table 10 and FIG. 6 indicate that all serotype 3 conjugates elicited dose dependent OPA titers in a murine immunogenicity model.

### Example 8: Evaluation of Immunogenicity of S. pneumoniae Serotype 3 (ST3 or Pn3) Glycoconjugates Using Different Chemistry and Different Carriers

Infant rhesus macaques, were vaccinated with Streptococcus pneumoniae serotype 3 (2 polysaccharide sizes; 250 kDa or 140 kDa) conjugated using Reductive Amination in aqueous (RAC/Aq.) or Reductive Amination in DMSO (RAC/DMSO) chemistry with 3 different carrier proteins (CRM₁₉₇, TT or SCP).

### 1. Infant Rhesus Monkey Studies

A total of 40 age and sex matched infant rhesus macaques (3-6 months old) were randomly divided in to 5 groups (see Study design at table 11). Three groups of ten infants were vaccinated intramuscular (right quadriceps) under sedation with S. pneumoniae serotype 3 conjugate prepared using RAC-DMSO at 2.2 μg/animal (all with a polysaccharide size of about 130 kDa) containing one of the carrier proteins—CRM₁₉₇, SCP or TT. Two control groups received S. pneumoniae serotype 3 conjugates prepared using RAC-aqueous (2.2 μg/animal) with CRM₁₉₇ as carrier and with a polysaccharide size of either about 250 kDa (n=6) or about 140 kDa (n=4). All animals received respective vaccines formulated with AlPO₄ at 125 μg/dose. Pre-bleeds to assess baseline ST3-specific sera titers were collected 1 week (wk=−1) before primary vaccination (DO). Two repeat vaccinations were administered on alternate legs at week 8 and week 16 following primary vaccination. Whole blood for sera was collected at 4- and 8-weeks post-dose 1(PD1); 1, 4 and 8 weeks after PD2 and 1, 4 and 16 weeks after PD3.

Frozen sera were thawed, heat inactivated and processed as described below to determine ST3 specific IgG and OPA titers.

### 2. Direct Luminex Immunoassays for Quantitation of Sera IgG

S. pneumoniae direct Luminex immunoassays (dLIAs) were used to quantify IgG antibody concentrations in serum for the following pneumo polysaccharides: 1, 3, 4, 5, 6A, 6B, 7F, 8, 9V, 10A, 11A, 12F, 14, 15B, 18C, 19A, 19F, 22F, 23F, and 33F. This was done using Luminex Magplex technology (see e.g. Pavliakova, Danka, et al. “Msphere 3.4 (2018): e00128-18).

### 3. Opsonophagocytosis Assay

Microcolony Opsonophagocytic Assay (mcOPA) was Performed.

For the Pn3 mcOPA, reaction mixtures composed of target bacterial cells and heat-inactivated test serum are incubated for 30 minutes at 25° C. in an environmental shaker. Differentiated HL-60 tissue culture cells (effector cells) and baby rabbit complement are then added to the reaction mixture, and incubated for 45 minutes at 37° C. in an environmental shaker. Functional anti-S. pneumoniae antibody titers are determined by measuring bacterial survival in mcOPA reactions containing the test serum. The assay mixture is plated and grown overnight.

On day 2, the number of non-phagocytosed live bacteria is determined. The mcOPA antibody titer is the reciprocal of the serum dilution resulting in 50% reduction in the number of bacterial colonies when compared to the bacteria-effector cell-complement control wells that do not contain serum.

TABLE 11

| Pn3 conjugate | | | Poly size | Degree of | |
|---|---|---|---|---|---|
| type | Chemistry | Carrier | (kDa) | activation | #IRM |
| CRM/RAC Aq. | RAC/Aq. | CRM₁₉₇ | 250 | 3.9 | 6 |
| CRM/RAC Aq. | RAC/Aq. | CRM₁₉₇ | 140 | 5 | 4 |
| CRM/RAC- | RAC-DMSO | CRM₁₉₇ | 130 | 14 | 10 |
| DMSO | | | | | |
| TT/RAC-DMSO | RAC-DMSO | TT | 130 | 14 | 10 |
| SCP/RAC- | RAC-DMSO | SCP | 130 | 14 | 10 |
| DMSO | | | | | |

### 4. Results

### 4.1. SCP Induced Higher OPA and IgG Geomean Titers Among Pn3 Conjugates Made Using RAC/DMSO:

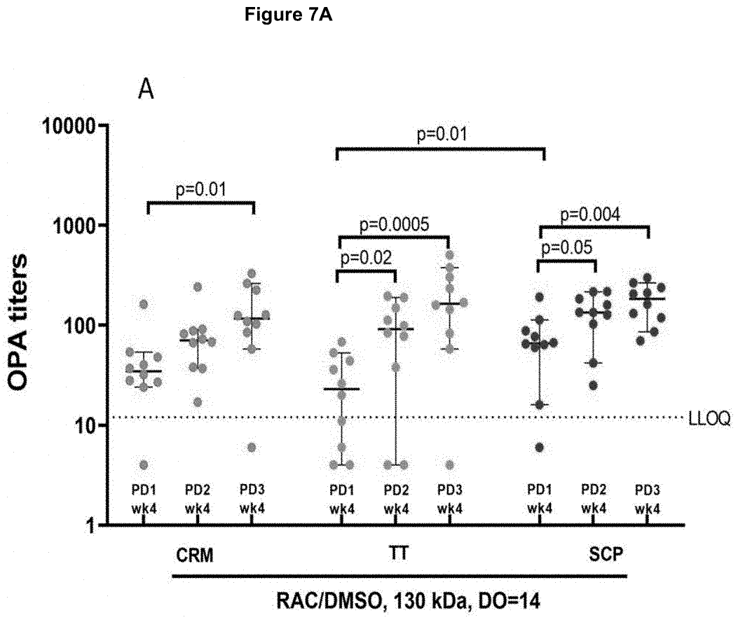

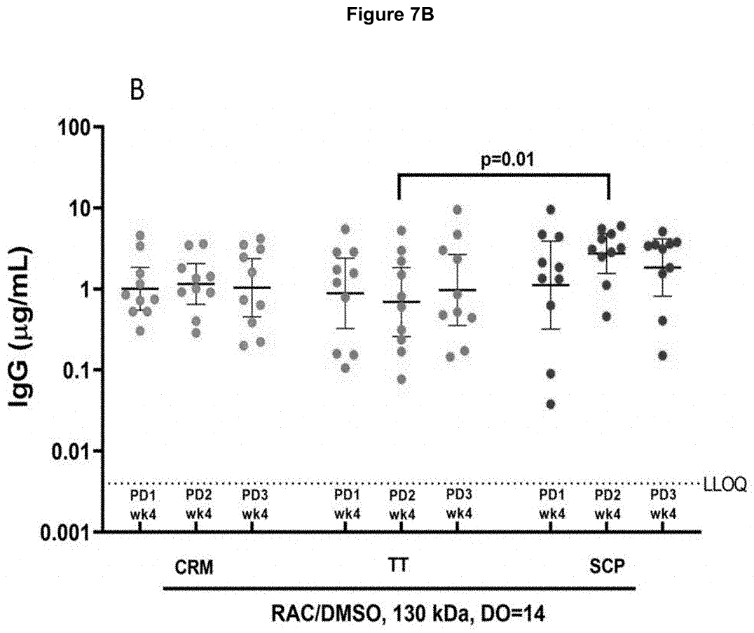

Following vaccination of infant rhesus macaques with Pn3 (RAC/DMSO) conjugated to -CRM₁₉₇, -TT, or -SCP, there was an increase in both OPA (FIG. 7A, Table 12) and IgG (FIG. 7B, Table 13) titers. Interestingly, Pn3 conjugated to SCP showed a 2 fold increase in IgG titers PD1 to PD2 (FIG. 7B, Table 13). Additionally, OPA titers did increase significantly with SCP and TT carriers at wk 4 following both second and third boost (FIG. 7A, Table 12). SCP induced higher average geomean titers at each of the post vaccination time point (FIG. 7A-B, Tables 12-13). A significant boosting effect between post dose 1 and 2 as well as dose 1 and 3 were observed with OPA titers in infant rhesus vaccinated with both TT or SCP carrier proteins (FIG. 7A, Table 12). However, SCP induced OPA titers with a narrow confidence interval indicating a uniform immune response generated compared to TT induced OPA titers (FIG. 7A, Table 12). CRM carrier induced a significant OPA booster response by dose 3 (FIG. 7A, Table 12). At dose 2, there was a significant increase in IgG response between TT and SCP carriers (FIG. 7B, Table 13)

TABLE 12
(FIG. 7A). OPA titers in Infant Rhesus Macaques

| Pn3 | | Geometric | | | |
|---|---|---|---|---|---|
| conjugate | Week post | mean OPA | Lower 95% | Upper 95% | |
| type | dose | titers | CI | CI | N |
| CRM/RAC- | Wk 4 PD1 | 32.80 | 17.01 | 63.27 | 10 |
| DMSO | Wk 4 PD2 | 64.58 | 39.15 | 106.6 | 10 |
| | Wk 4 PD3 | 100.25 | 44.87 | 224 | 10 |
| TT/RAC- | Wk 4 PD1 | 17.76 | 8.25 | 38.24 | 10 |
| DMSO | Wk 4 PD2 | 54.81 | 19.30 | 155.7 | 10 |
| | Wk 4 PD3 | 125.94 | 46.96 | 337.8 | 10 |
| SCP/RAC- | Wk 4 PD1 | 54.58 | 26.76 | 111.3 | 10 |
| DMSO | Wk 4 PD2 | 113.11 | 68.05 | 188.0 | 10 |
| | Wk 4 PD3 | 162.01 | 114.3 | 229.6 | 10 |

TABLE 13
(FIG. 7B). IgG titers in Infant Rhesus Macaques

| Pn3 | | Geometric | | | |
|---|---|---|---|---|---|
| conjugate | Week post | mean IgG | Lower 95% | Upper 95% | |
| type | dose | (ug/mL) | CI | CI | N |
| CRM/RAC- | Wk 4 PD1 | 1.01 | 0.54 | 1.84 | 10 |
| DMSO | Wk 4 PD2 | 1.15 | 0.64 | 2.06 | 10 |
| | Wk 4 PD3 | 1.04 | 0.45 | 2.36 | 10 |
| TT/RAC- | Wk 4 PD1 | 0.88 | 0.32 | 2.39 | 10 |
| DMSO | Wk 4 PD2 | 0.69 | 0.25 | 1.83 | 10 |
| | Wk 4 PD3 | 0.97 | 0.35 | 2.65 | 10 |
| SCP/RAC- | Wk 4 PD1 | 1.11 | 0.31 | 3.88 | 10 |
| DMSO | Wk 4 PD2 | 2.74 | 1.55 | 4.82 | 10 |
| | Wk 4 PD3 | 1.84 | 0.81 | 4.14 | 10 |

4.2. OPA and IgG Titers Carriers with High (250 kDa) and Lower (140 kDa) Polysaccharide Size Using RAC/Aq.:

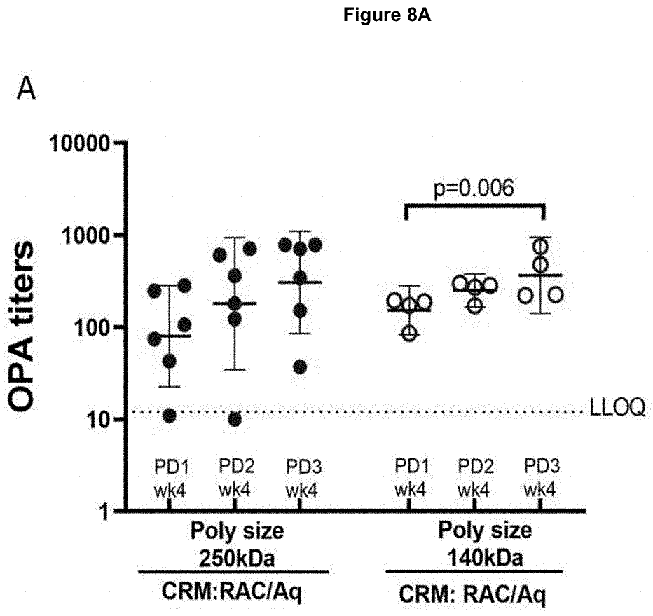

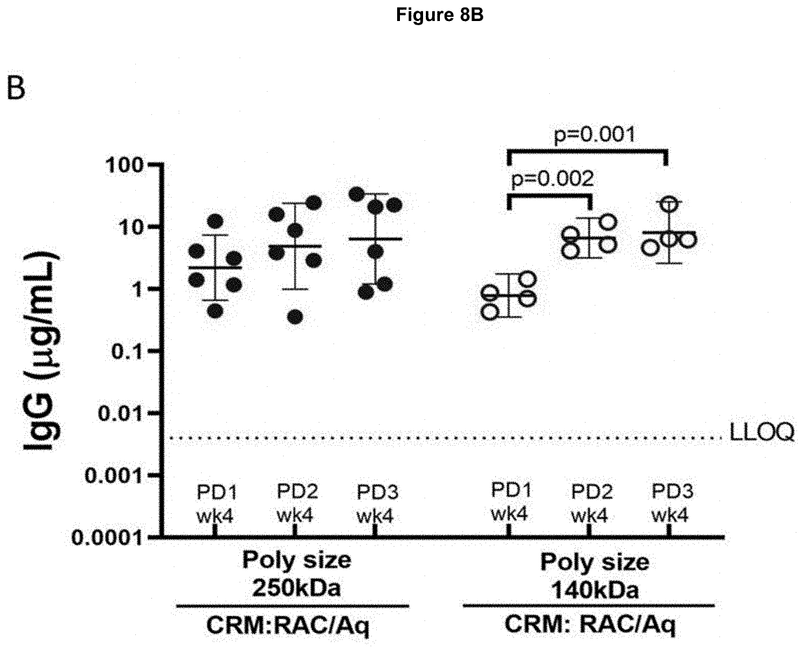

Results are shown at FIG. 8A-8B and Tables 14-15. A significant boosting effect with OPA and IgG titers were observed between dose 1 and 3 in Rac/Aq 140 kDa group (FIG. 8A-B). IgG titers were also significantly improved with dose 2 of Rac/Aq 140 kDA (FIG. 8B).

TABLE 14
(FIG. 8A). OPA titers in Infant Rhesus Macaques

| Pn3 | | Geometric | | | |
|---|---|---|---|---|---|
| conjugate | Week post | mean OPA | Lower 95% | Upper 95% | |
| type | dose | titers | CI | CI | N |
| CRM/RAC Aq. | Wk 4 PD1 | 79.95 | 22.51 | 284.0 | 6 |
| (250 kDa) | Wk 4 PD2 | 180.46 | 34.68 | 939.2 | 6 |
| | Wk 4 PD3 | 306.80 | 85.28 | 1104.0 | 6 |
| CRM/RAC Aq. | Wk 4 PD1 | 152.62 | 82.65 | 281.8 | 4 |
| (140 kDa) | Wk 4 PD2 | 251.12 | 166.2 | 379.4 | 4 |
| | Wk 4 PD3 | 365.55 | 141.4 | 945.1 | 4 |

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. A method of making a Streptococcus pneumoniae serotype 3 glycoconjugate, comprising the steps of:
(a) reacting an isolated Streptococcus pneumoniae serotype 3 capsular polysaccharide with an oxidizing agent;
(b) compounding the activated polysaccharide of step (a) with a carrier protein; and
(c) reacting the compounded activated polysaccharide and carrier protein with a reducing agent to form a glycoconjugate, wherein the isolated polysaccharide is sized before the activation step (a) to a weight average molecular weight between 100 kDa and 200 kDa and wherein the reduction reaction (c) is carried out in aprotic solvent.

2. The method of claim 1 wherein the oxidizing agent is periodate or periodic acid.

3. The method of claim 1 wherein the degree of oxidation of the activated serotype 3 polysaccharide is between 11 to 19.

4. (canceled)

5. The method of claim 1 wherein the initial input ratio (weight by weight) of activated serotype 3 capsular polysaccharide to carrier protein is between 1.5:1 and 0.5:1.

6. The method of claim 1 wherein the reduction reaction (c) is carried out in a solution consisting essentially of dimethylsulphoxide (DMSO).

7. The method of claim 1 wherein the reducing agent is sodium cyanoborohydride.

8. The method of claim 1 wherein, the product of step (c) is reacted with 1 to 20 molar equivalents of sodium borohydride for 15 mins-15 hrs.

9. The method of claim 1 further comprising the step of purifying the glycoconjugate after it is produced.

10. A Streptococcus pneumoniae serotype 3 glycoconjugate produced according to the method of claim 1.

11. The glycoconjugate of claim 10 comprising a serotype 3 capsular polysaccharide wherein the weight average molecular weight (Mw) of said polysaccharide before conjugation is between 120 kDa and 180 kDa.

12. (canceled)

13. The glycoconjugate of claim 10 having a weight average molecular weight (Mw) of between 1,000 kDa and 5,000 kDa.

14. (canceled)

15. The glycoconjugate of claim 10 wherein the degree of conjugation of said glycoconjugate is between 2 and 15.

16. The glycoconjugate of claim 10 wherein the saccharide to carrier protein ratio (w/w) is between 0.9 and 1.1.

17. The glycoconjugate of claim 10 wherein said carrier protein is CRM₁₉₇, SCP or an enzymatically inactive fragment of SCP comprising SEQ ID NO: 41 or SEQ ID NO: 42.

18-19. (canceled)

20. An immunogenic composition comprising a Streptococcus pneumoniae serotype 3 glycoconjugate of claim 10.

21. The immunogenic composition of claim 20 comprising from 1 to 25 glycoconjugates from different serotypes of S. pneumoniae.

22. The immunogenic composition of claim 20 further comprising glycoconjugates from S. pneumoniae serotypes 1, 4, 5, 6A, 6B, 7F, 8, 9V, 10A, 11A, 12F, 14, 15B, 18C, 19A, 19F, 22F, 23F and 33F, wherein said immunogenic composition is a 20-valent pneumococcal conjugate composition.

23. The immunogenic composition of claim 20 further comprising glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein said immunogenic composition is a 25-valent pneumococcal conjugate composition.

24. The immunogenic composition of claim 20 further comprising glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38, wherein said immunogenic composition is a 25-valent pneumococcal conjugate composition.

25. A method of making a Streptococcus pneumoniae serotype 3 glycoconjugate, comprising the steps of:
(a) reacting an isolated Streptococcus pneumoniae serotype 3 capsular polysaccharide with 1,1′-carbonyldiimidazole (CDI) or 1,1′-Carbonyl-di-(1,2,4-triazole) (CDT) in an aprotic solvent;
(b) reacting the activated polysaccharide of step (a) with a carrier protein in an aprotic solvent to form a glycoconjugate.

26. The method of claim 25 wherein, the isolated polysaccharide is sized to a weight average molecular weight between 100 kDa and 200 kDa.

27. The method of claim 25 wherein, step La) comprises reacting the polysaccharide with an amount of CDI or CDT that is between 0.01-10 molar equivalent to the amount of serotype 3 capsular polysaccharide present in the reaction mixture.

28. The method of claim 25 wherein, the activating reaction La), the conjugation reaction (b), or both, is carried out in a solution consisting essentially of dimethylsulphoxide (DMSO) or dimethylformamide (DMF).

29. The method of claim 25 wherein, the activating reaction La) is carried out in an aprotic solvent comprising 0.1% to 1% water.

30. (canceled)

31. The method of claim 25 wherein, the conjugation reaction (b) is carried out in DMSO comprising about 0.1% to about 10% v/v water.

32. The method of claim 25 wherein a weak organic base is added to the reaction mixture after the activating reaction La) but before the conjugation reaction (b).

33. The method of claim 32 wherein said weak organic base is selected from alkanamines, imidazole, triazole, pyridine, histidine and guanidine.

34. The method of claim 25 further comprising the step of purifying the glycoconjugate after it is produced.
